(19) United States
(12) Patent Application Publication
(10) Pub. No.: US 2023/0042103 A1
(43) Pub. Date: Feb. 9, 2023

(54) ENGINEERED NUCLEIC ACID REGULATORY ELEMENT AND METHODS OF USES THEREOF

(71) Applicant: REGENXBIO INC., Rockville, MD (US)

(72) Inventors: Chunping Qiao, Rockville, MD (US); Devin McDougald, Boston, MA (US)

(21) Appl. No.: 17/628,517

(22) PCT Filed: Jul. 24, 2020
(86) PCT No.: PCT/US2020/043578

§ 371 Date: Jan. 19, 2022

Related U.S. Application Data
(60) Provisional application No. 62/879,358, filed on Jul. 26, 2019.
(60) Provisional application No. 62/967,480, filed on Jan. 29, 2020.
(60) Provisional application No. 63/023,152, filed on May 11, 2020.

(51) Int. Cl. C12N 15/86

(52) U.S. Cl. CPC C12N 15/86; C12N 2750/14143; C12N 2830/007; C12N 2830/008

(57) ABSTRACT

The present invention relates to nucleic acid expression cassettes that are engineered to enhance gene expression. Vectors and methods employing the expression cassettes containing novel chimeric regulatory elements are provided. The invention is particularly useful for delivery of transgenes to target cells and confers desirable properties for liver-directed and muscle-directed or liver-directed and bone-directed gene therapy. Moreover, the invention relates to a novel method of engineering tandem enhancer/promoter elements and expressing transgenes for example within liver and/or muscle cells, and delivery of therapeutics for treating various disorders.

### 1. FIELD OF THE INVENTION

The present invention relates to nucleic acid regulatory elements engineered to enhance gene expression, methods of employing the regulatory elements and uses thereof. Use of the engineered regulatory elements upstream of a transgene delivered to target cells confers desirable properties, and in some cases confers desirable properties for gene therapy. In particular, the invention provides nucleic acid regulatory elements operably linked to a heterologous gene (transgene) inserted into an expression cassette, such that the regulatory elements drive expression of the transgene in specific cells. As such, the invention also provides a method to target tissues, in particular, expression cassettes comprising the engineered regulatory elements improve expression of the transgene in liver and/or muscle, including heart, tissue or in liver and/or bone tissue, as well as deliver therapeutics systemically for the treatment of various disorders. Moreover, the invention relates to a novel method of engineering tandem enhancer/promoter elements and expressing transgenes specifically within liver and/or muscle cells.

### 2. BACKGROUND

The use of regulatory elements to drive gene expression is highly complex. Both naturally occurring and synthetic regulatory elements, such as enhancers and promoters, have been reported in the art. It is not known whether multiple elements engineered for heterologous gene expression will produce various aberrant, unstable and/or competing transcripts in a given tissue environment.

Gene expression vectors that are highly productive and stable may be suitable for gene therapy. Transgenes delivered with AAV or other viral vectors aim to provide long-term gene expression and thus may boost systemic expression levels or serum half-life of a biotherapeutic transgene. As such, improved gene expression systems for gene therapy would greatly benefit patients compared to direct injection of a biologic drug, such as in enzyme replacement therapy. Although AAV capsid proteins that carry genome DNA can confer a particular tissue tropism to deliver DNA into target cells, it is desirable to express greater amounts of the gene of interest in liver, due to its low immunogenicity (Pastore, et al. Human Gene Therapy Vol. 10, No. 11, July 1999 online ahead of print).

Thus, liver and muscle, including, heart, or liver and bone expression of a biotherapeutic would be desirable to elevate serum levels and systemic delivery of the protein. There remains a need for tissue-targeted gene expression and vectors that are highly productive in liver, bone, heart and/or skeletal muscle.

### 3. SUMMARY OF THE INVENTION

Provided are recombinant expression cassettes comprising a composite nucleic acid regulatory element for enhancing or directing gene expression in the liver and, in certain embodiments, also muscle, which includes skeletal muscle and, in embodiments, may also include heart or cardiac muscle, or bone tissue, comprising at least two enhancers and at least two promoters, particularly those listed in Table 1, operably linked to a transgene. In some embodiments, the composite nucleic acid regulatory element comprises two promoters arranged in tandem where the downstream or 3′ promoter is start codon-modified (for example, deleted for the start codon (ΔATG)).

Provided are recombinant expression cassettes comprising a composite nucleic acid regulatory element which comprises two promoters arranged in tandem, wherein one of the promoters is an hAAT promoter and, in certain embodiments, the hAAT promoter is the downstream promoter in the arrangement and is start-codon modified (that is deleted for the start codon or ΔATG), wherein the composite nucleic acid regulatory element is operably linked to a transgene. In some embodiments, the composite nucleic acid regulatory element comprises two promoters arranged in tandem, wherein one of the promoters is TBG and, in embodiments, the TBG promoter is the downstream promoter and is start-codon modified (ΔATG), wherein the nucleic acid regulatory element is operably linked to a transgene. In certain embodiments, the second promoter is an hAAT promoter, a TBG promoter, a CK8 promoter, an Spc5.12 promoter, a minSpc5.12 promoter, a Sp7/Osx promoter or a minSp7/Osx. The composite nucleic acid regulatory element further comprises one or more enhancer elements, including one or two copies of the ApoE enhancer (including two copies arranged in tandem), one or two copies of the Mic/Bike (including two copies arranged in tandem), one or two copies of MckE (including two copies arranged in tandem), or a copy of MhcE and MckE arranged in tandem.

In some embodiments, the composite nucleic acid regulatory element comprises a) two copies in tandem of Mic/BiKE, two copies in tandem of ApoE enhancer, two or three copies in tandem of MckE, or one copy of MhcE in tandem with one copy of MckE, b) one promoter or, in an embodiment, two promoters arranged in tandem wherein at least one promoter is the hAAT promoter (in embodiments, the 3′ promoter) and it is, optionally, start-codon modified or deleted (ΔATG), and the composite nucleic acid regulatory element is operably linked to a transgene. In some embodiments, the composite nucleic acid regulatory element comprises LSPX1, LSPX2, LTP1, LTP2, or LTP3 of Table 1, and the composite nucleic acid regulatory element is operably linked to a transgene. In some embodiments, the composite nucleic acid regulatory element comprises LMTP6, LMTP13, LMTP14, LMTP15, LMTP18, LMTP19, or LMTP20 of Table 1, and the composite nucleic acid regulatory element is operably linked to a transgene. In some embodiments, the composite nucleic acid regulatory element comprises LBTP1 or LBTP2 of Table 1, and the composite nucleic acid regulatory element is operably linked to a transgene. The transgene may be any one of the genes or nucleic acids encoding the therapeutic proteins listed in, but not limited to, Tables 4A-4D. In certain embodiments, the transgene encodes a therapeutic antibody, either having full length heavy and light chains or an antigen binding fragment, such as a Fab fragment.

Provided are composite nucleic acid regulatory elements for enhancing and/or directing gene expression in the liver comprising nucleic acid sequences SEQ ID NO:1, SEQ ID NO:2, SEQ ID NO:3, SEQ ID NO:4, SEQ ID NO:5, SEQ ID NO:6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31.

Also provided are vectors comprising an expression cassette comprising more than one (for example, 2, 3 or 4) Mic/BiK enhancer sequence, Mck enhancer sequence, or MhcE sequence or one or more ApoE (one, two or three ApoE sequence) upstream of (that is 5′ of) more than one tissue-specific promoter, and, optionally, the downstream tissue-specific promoter (i.e., the tissue-specific promoter closest to the transgene) but not the first tissue-specific promoter (i.e., the most 5′ tissue specific promoter) is start codon modified (ΔATG). In some embodiments, the expression cassette directs expression of the transgene in target tissues, e.g. the transgene listed in, but not limited to, Tables 4A-4D, including a therapeutic antibody, such as a full length antibody or antigen binding fragment, such as a Fab fragment. In some embodiments, the vectors comprise a transgene operably linked to a composite nucleic acid regulatory element comprising or consisting of a nucleic acid sequence of SEQ ID NO:1, SEQ ID NO:2, SEQ ID NO:3, SEQ ID NO:4, SEQ ID NO:5, SEQ ID NO:6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31. In some embodiments, the vectors comprise a transgene operably linked to a composite nucleic acid regulatory element comprising or consisting of a nucleic acid sequence of SEQ ID NO:1, SEQ ID NO:2, SEQ ID NO:3, SEQ ID NO:4, SEQ ID NO:5, SEQ ID NO:6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31, wherein the transgene is expressed in the liver following administration to the subject. In some embodiments, the vectors comprise a transgene operably linked to a composite nucleic acid regulatory element comprising or consisting of a nucleic acid sequence of SEQ ID NO:1, SEQ ID NO:2, SEQ ID NO:3, SEQ ID NO:4, or SEQ ID NO:5, wherein the transgene is expressed in greater amounts in the liver (for example, more protein per viral genome detected) than in the muscle following administration to the subject. In some embodiments, the vectors comprise a transgene operably linked to a composite nucleic acid regulatory element comprising or consisting of a nucleic acid sequence of SEQ ID NO:6, SEQ ID NO:21, SEQ ID NO:22, SEQ ID NO:23, SEQ ID NO:24, SEQ ID NO:25, or SEQ ID NO:26, wherein the transgene is expressed in both the liver and the muscle, including skeletal muscle, as well as, in embodiments, in the heart, following administration to the subject. In some embodiments, the vectors comprise a transgene operably linked to a composite nucleic acid regulatory element comprising or consisting of a nucleic acid sequence of SEQ ID NO: 30 or SEQ ID NO: 31, wherein the transgene is expressed in both the liver and the bone following administration to the subject.

Also provided are methods for enhancing expression of a transgene, comprising delivery of viral vectors comprising nucleic acid expression cassettes comprising a 5′ to 3′ arrangement of a) more than one, for example, two or three sequences, selected from Mic/BiK enhancer sequences, ApoE enhancer sequences, Mck enhancer sequences, or Mhc enhancer sequences b) one or more, for example two, liver-specific promoters, wherein at least one liver-specific promoter, for example one liver-specific promoter, comprises a modified start codon, and c) a transgene. In some embodiments, provided are viral vectors incorporating the engineered expression cassettes described herein, including rAAVs. sequences

Also provided are methods for enhancing expression of a transgene, comprising delivery of viral vectors comprising nucleic acid expression cassettes comprising a 5′ to 3′ arrangement of a) one or more, for example, two or three, ApoE enhancer, b) one or more, for example two, bone-specific promoters, such as an Sp7 or Sp7/Osx promoter, c) one or more, for example, two, liver-specific promoters, optionally wherein at least one liver-specific promoter, for example one liver-specific promoter, comprises a modified start codon, and d) a transgene.

In some embodiments, provided are viral vectors incorporating the engineered expression cassettes described herein, including rAAVs.

In another aspect, a method of treatment by delivery of rAAVs comprising the nucleic acid expression cassettes described herein are also provided. A method for treating a disease or disorder in a subject in need thereof comprising the administration of recombinant AAV particles comprising an expression cassette having more than one, for example two or three, Mic/BiK enhancer sequences, or ApoE enhancer sequences, or Mck enhancer sequences upstream of one or more, for example two, liver-specific promoters, wherein at least one liver-specific promoter comprises a modified start codon, and a transgene, is provided. Also provided is a method for treating a disease or disorder in a subject in need thereof comprising the administration of recombinant AAV particles comprising an expression cassette having one or more ApoE enhancer sequences, one or more bone-specific promoters, such as an Sp7 or Sp7/Osx promoter, one or more liver-specific promoters wherein at least one liver-specific promoter comprises a modified start codon, and a transgene is provided.

Also provided are methods of producing recombinant AAV vectors comprising an expression cassette with AAV comprising a composite nucleic acid regulatory element described herein operably linked to a transgene by culturing a host cell comprising an artificial genome flanked by AAV ITRs and comprising the nucleic acid regulatory element operably linked to the transgene and a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans; sufficient adenovirus helper functions to permit replication and packaging of the artificial genome by the AAV capsid protein; and recovering recombinant AAV encapsidating the artificial genome from the cell culture. Host cells for production of the recombinant AAV described herein are also provided.

The invention is illustrated by way of examples infra describing the construction and function of gene cassettes engineered with composite regulatory elements designed on the basis of several liver-specific enhancers and promoters, in tandem with or without muscle (skeletal and/or cardiac)-specific or bone-specific enhancers and promoters, whereas the downstream elements are modified at their translation start sites.

### 4. BRIEF DESCRIPTION OF THE FIGURES

FIG. 1 depicts construction of recombinant nucleic acid regulatory elements, particularly promoter and enhancer sequences for use in expression cassettes.

FIG. 2 depicts relative fluorescence as a measure of GFP expression in liver cells. All of the constructs having LSPX1, LSPX2, LTP1, LTP2, and LTP3 promoters maintain liver specificity.

FIG. 3 depicts relative fluorescence as a measure of GFP expression in muscle cells. Dual specificity construct LMTP6 maintains muscle specificity, whereas LSPX1, LSPX2, LTP1, LTP2, and LTP3 promoters do not appear to promote expression in muscle cells.

FIG. 4 depicts GFP expression driven by the promoters of FIG. 1 in liver cells.

FIG. 5 depicts construction of recombinant nucleic acid regulatory elements, particularly dual specificity promoter LMTP6.

FIG. 6 depicts relative fluorescence as a measure of GFP expression in liver cells. Dual specificity construct LMTP6 maintains liver specificity, whereas CK8 control promoter is silent.

FIG. 7 depicts various measures of GFP expression driven by the LMTP6 promoter in liver cells.

FIGS. 8A and 8B depict construction of recombinant nucleic acid regulatory elements, particularly promoter and enhancer sequences for use in expression cassettes, particularly liver/muscle (A) and liver/bone (B) dual specificity regulatory elements.

FIGS. 9A and 9B depict GFP expression driven by different regulatory elements in a muscle-cell derived cell line, C2C12. (A) The activity, as eGFP intensity, of three liver/muscle dual-specific regulatory elements (LMTP13, LMTP14, and LMPT15) is shown in comparison to the muscle-specific promoters Spc5.12 and minSpc5.12. The liver-specific promoter hAAT and negative control are also shown. (B) The activity (GFP expression level) of four liver/muscle dual-specific regulatory elements (LMTP6, LMTP18, LMTP19, and LMPT20) is shown in comparison to the muscle-specific promoters CK8 and the liver-specific promoter hAAT.

FIG. 10 depicts the expression of an anti-plasma kallikrein antibody (pKal, referred to as “Mab1” herein) in C2C12 muscle cells upon transduction of the cells with different cis plasmids expressing the monoclonal antibody under the control of different regulatory elements: CAG, LMTP6, and hAAT. For detection of antibody protein, following transduction, the cells were treated with FITC conjugated anti-Fc (IgG) antibody. DAPI staining is shown to confirm confluency and viability of the cells under all conditions tested.

FIGS. 11A and 11B. A Serum expression levels (μg/ml) of a pKal antibody (Mab1) upon intravenous injection of C/57BL6 mice with 2.5×10¹² vg/kg of AAV8 vectors encoding Mab1 regulated by different liver-specific, liver-tandem and liver-muscle regulatory elements (see Table 1). CAG (SEQ ID NO: 17) and TBG (SEQ ID NO: 10) promoters were used as controls. Data from the blood draw at 1, 3, 5 and 7 weeks post injection are shown. LSPX1, liver-specific promoter 1 (SEQ ID NO: 1); LSXP2, liver-specific promoter 2 (SEQ ID NO: 2); LTP1, liver-specific tandem promoter 1 (SEQ ID NO: 3); LMTP6, liver and muscle dual-specific tandem promoter 6 (SEQ ID NO: 6). Protein expression levels were quantified by ELISA from biweekly serum collections. N=5 mice per vector. Numbers on x-axis represent the weeks post vector administration. Data represent mean+SEM. B. Quantification of viral genomes in liver. C57Bl/6 mice were administrated intravenously with AAV8 vectors driven by different liver-specific, liver-tandem and liver-muscle regulatory elements (see Table 1; CAG (SEQ ID NO: 17) and TBG (SEQ ID NO: 10) promoters were used as controls) at equivalent doses (2.5×10¹² vg/kg). N=5 mice per group. Vector DNA was analyzed by ddPCR in mouse liver samples collected at 49 days post vector administration. Data represent mean+SEM.

FIGS. 12A and 12B. A shows serum expression levels (μg/ml) of an anti-plasma kallikrein (pKal) antibody (Mab1) upon intravenous injection of C/57BL6 mice with 2.5E12 gc/kg of AAV8 vectors encoding Mab1 under the control of different regulatory elements: CAG, LMTP6, and hAAT. CAG promoter was used as control. Data from the blood draw at 7, 21, and 35 days post injection are shown. B shows transgene expression from liver and heart upon intravenous injection of C/57BL6 mice with 2.5E12 gc/kg of AAV8 vectors encoding Mab1 under the control of different regulatory elements: CAG, LMTP6, and hAAT. Quantification was done with ddPCR analysis of anti-pKal mRNA copies and normalized to GAPDH across tissues.

FIGS. 13A-13D. A. Serum anti-kallikrein (pKal) antibody (Mab1) concentration following AAV8 delivery to mice where the pKal antibody expression is under control of CAG, hAAT or LMTP6 promoter. Animals received bilateral injections of 5×10¹⁰ vg/kg into the GA muscle. Serum was collected biweekly and pKal antibody concentration was quantified with ELISA. B. Vector genome quantification from GA muscle, liver and heart tissue with digital droplet PCR (ddPCR). C. Comparison of pKal antibody expression from liver as mRNA. Data represent relative fold gene expression (mRNA levels) as quantified by the AACT method. D. Comparison of AAV transgene expression from GA muscle, liver and heart tissues using digital droplet PCR (ddPCR). Anti-pKal antibody mRNA copies were normalized to GAPDH mRNA copies across tissues. Data are represented as mean±SEM. Statistical significance was determined using a one-way ANOVA followed by Tukey's HSD post-test. *P<0.05, **P<0.01.

FIG. 14 depicts the expression level of the secreted protein precursor of a lysosomal enzyme upon transduction of Huh-7 cells (hepatocytes) with different constructs comprising indicated promoters. V1, transgene sequence was codon-optimized; V2, transgene sequence was codon-optimized and CpG depleted.

FIG. 15 illustrates a flowchart for processing and analyzing sequence data via open source data analysis tools through usegalaxy.org and final tabulation of reads by their start location using standard calculation and data sorting tools.

FIGS. 16A-16F depict various promoter constructs and their transcriptional start sites in liver or muscle cells as determined by RACE and NGS.

### 5. DETAILED DESCRIPTION

The inventors have provided, in part, unique combinations of promoter and enhancer sequences in expression cassettes suitable for improvement of transgene expression while maintaining or conferring tissue specificity. Provided are vectors, such as viral vectors, incorporating the engineered expression cassettes described herein, including rAAVs, for use in therapy, and methods and host cells for producing same. The novel regulatory element nucleic acids were generated by a new method to improve transgene expression from tandem promoters (i.e. two promoter sequences driving expression of the same transgene) by depleting the 3′ promoter sequence of potential ‘ATG’ initiation sites. This approach was employed to improve transgene expression from tandem tissue-specific promoter cassettes (such as those targeting the liver) as well as promoter cassettes to achieve dual expression in at least two separate tissue populations (such as liver and skeletal muscle and/or cardiac muscle; or liver and bone). Ultimately, these designs may improve the therapeutic efficacy of gene transfer by providing more robust levels of transgene expression, improved stability/persistence, and induction of immune tolerance to the transgene product.

### 5.1. Definitions

The term “regulatory element” or “nucleic acid regulatory element” are non-coding nucleic acid sequences that control the transcription of neighboring genes. Cis regulatory elements typically regulate gene transcription by binding to transcription factors. This includes “composite nucleic acid regulatory elements” comprising more than one enhancer or promoter elements as described herein.

The term “expression cassette” or “nucleic acid expression cassette” refers to nucleic acid molecules that include one or more transcriptional control elements including, but not limited to promoters, enhancers and/or regulatory elements, introns and polyadenylation sequences. The enhancers and promoters typically function to direct (trans)gene expression in one or more desired cell types, tissues or organs.

The term “operably linked” and “operably linked to” refers to nucleic acid sequences being linked and typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked and still be functional while not directly contiguous with a downstream promoter and transgene.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into or modification of the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

The terms “subject”, “host”, and “patient” are used interchangeably. As used herein, a subject is preferably a mammal such as a non-primate (e.g., cows, pigs, horses, cats, dogs, rats etc.) or a primate (e.g., monkey and human), most preferably a human.

The terms “therapeutic agent” or “biotherapeutic agent” refer to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The phrase “liver-specific” or “liver-directed” refers to nucleic acid elements that have adapted their activity in liver (hepatic) cells or tissue due to the interaction of such elements with the intracellular environment of the hepatic cells. The liver acts as a bioreactor or “depot” for the body in the context of a gene therapy delivered to the liver tissue and a gene cassette enhanced for expression in the liver will produce the biotherapeutic (translated protein) that is secreted into the circulation. As such, the biotherapeutic agent is delivered systemically to the subject by way of liver expression. Without being bound by any one theory, liver production of a biotherapeutic agent (such as produced by the delivered transgene) can provide immunotolerance to the agent such that endogenous T cells of the subject producing the protein will recognize the protein as self-protein, and not induce an innate immune response.

The phrase “bone-specific” or “bone-directed” refers to nucleic acid elements that have adapted their activity in bone cells (e.g. osteoblasts, osteoclasts, osteocytes and bone lining cells) or tissue including various types of cells and collagenous extracellular organic matrix due to the interaction of such elements with the intracellular environment of the bone cells. Secretion of transgene product into the bone, muscle and/or bloodstream may be enhanced following various routes of rAAV administration, such as intravenous or intramuscular administration, due to expression in bone where bone-specific promoters are present. Various therapeutics benefit from bone-specific expression of the transgene, or from both bone-specific and liver-specific expression of the transgene. Bone production of a biotherapeutic agent (such as produced by the delivered transgene) may provide also provide the host with increased immunotolerance to the agent, as compared to direct injection of an equivalent protein agent to the host.

The phrase “muscle-specific” or “muscle-directed” refers to nucleic acid elements that have adapted their activity in muscle cells or tissue due to the interaction of such elements with the intracellular environment of the muscle cells. Muscle cells include skeletal muscle as well as cardiac muscle. Secretion of transgene product into the muscle, and/or bloodstream may also be enhanced following various routes of administration, such as intravenous or intramuscular administration, due to intramuscular expression where muscle-specific promoters are present. Various therapeutics benefit from muscle-specific expression of the transgene, or from both muscle-specific and liver-specific expression of the transgene. Muscle production of a biotherapeutic agent (such as produced by the delivered transgene) may provide also provide the host with increased immunotolerance to the agent, as compared to direct injection of an equivalent protein agent to the host.

### 5.2. Regulatory Elements

One aspect relates to nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

Provided are arrangements of combinations of nucleic acid regulatory elements that promote transgene expression in liver and muscle (skeletal and/or cardiac) tissue or liver and bone tissue. In particular, certain elements are arranged with two or more copies of the individual enhancer and promoter elements arranged in tandem and operably linked to a transgene to promote expression, particularly tissue specific expression. Exemplary nucleotide sequences of the individual promoter and enhancer elements are provided in Table 1. Also provided in Table 1 are exemplary composite nucleic acid regulatory elements comprising the individual tandem promoter and enhancer elements. In certain embodiments the downstream promoter is an hAAT promoter (in certain embodiments the hAAT promoter is an hAAT(ΔATG) promoter) and the other promoter is another hAAT promoter or is a TBG promoter).

Accordingly, with respect to liver and muscle specific expression, provided are nucleic acid regulatory elements that comprise or consist of promoters and/or other nucleic acid elements, such as enhancers, that promote liver expression, such as ApoE enhancers, Mic/BiKE elements or hAAT promoters. These may be present as single copies or with two or more copies in tandem. The nucleic acid regulatory element may also comprise, in addition to the one or more elements that promote liver specific expression, one or more elements that promote muscle specific expression (including skeletal and/or cardiac muscle), for example, one or more copies, for example two copies, of the MckE element, which may be arranged as two or more copies in tandem or an MckE and MhcE elements arranged in tandem. In certain embodiments, a promoter element is deleted for the initiation codon to prevent translation initiation at that site, and preferably, the element with the modified start codon is the promoter that is the element at the 3′ end or the downstream end of the nucleic acid regulatory element, for example, closest within the nucleic acid sequence of the expression cassette to the transgene. In certain embodiments, the composite nucleic acid regulatory element comprises an hAAT promoter, in embodiments an hAAT which is start-codon modified (ΔATG) as the downstream promoter, and a second promoter in tandem with the hAAT promoter, which is an hAAT promoter, a CK8 promoter, an Spc5.12 promoter or an minSpc5.12 promoter.

The recombinant expression cassettes provided herein comprise i) a composite nucleic acid regulatory element comprising a) two copies of Mic/BiKE arranged in tandem or two copies of ApoE arranged in tandem or two copies of Mic/BiKE arranged in tandem with one copy of ApoE, b) one promoter or, in tandem promoter embodiments, two promoters arranged in tandem comprising at least one copy of hAAT which is start-codon modified (ΔATG) (where in certain embodiments the hAAT promoter is the downstream or 3′ promoter), and ii) a transgene, to which the composite nucleic acid regulatory element is operably linked. In some embodiments, the composite nucleic acid regulatory element comprises LSPX1, LSPX2, LTP1, LTP2, or LTP3 of Table 1. In some embodiments, the composite nucleic acid regulatory element is operably linked to a transgene. The transgene may be any one of the genes or nucleic acids encoding the therapeutic proteins listed in, but not limited to, Tables 4A-4D. The transgene may also encode a therapeutic antibody, including a full length antibody or an antigen binding fragment, such as a Fab fragment.

The recombinant expression cassettes provided herein comprise i) a nucleic acid regulatory element comprising a) one copy of ApoE, two or three copies of MckE arranged in tandem, one copy of each MckE, MhcE, and ApoE arrange in tandem, or two or three copies of MckE arranged in tandem with one copy of ApoE, b) two copies of a promoter arranged in tandem comprising at least one copy of hAAT which is start-codon modified (ΔATG), and ii) a transgene. In certain embodiments, the second and upstream promoter is a CK8 promoter, an Spc5.12 promoter or a minSpc5.12 promoter. In some embodiments, the composite nucleic acid regulatory element comprises LMTP6, LMTP13, LMTP14, LMTP15, LMTP18, LMTP19, or LMTP20 of Table 1. In some embodiments, the composite nucleic acid regulatory element is operably linked to a transgene. The transgene may be any one of the genes or nucleic acids encoding the therapeutic proteins listed in, but not limited to, Tables 4A-4D. In certain embodiments, the transgene is a therapeutic antibody, including a full length antibody or antigen binding fragment thereof, such as, a Fab fragment.

Provided are composite nucleic acid regulatory elements for enhancing gene expression in the liver comprising nucleic acid sequences SEQ ID NO:1, SEQ ID NO:2, SEQ ID NO:3, SEQ ID NO:4, SEQ ID NO:5, SEQ ID NO:6, SEQ ID NO:21, SEQ ID NO:22, SEQ ID NO:23, SEQ ID NO:24, SEQ ID NO:25, SEQ ID NO: 26, SEQ ID NO: 30, or SEQ ID NO:31. Also included are composite regulatory elements that enhance gene expression in the liver, and in certain embodiments, also muscle or bone, which have 99%, 95%, 90%, 85% or 80% sequence identity with one of nucleic acid sequences SEQ ID NO:1, SEQ ID NO:2, SEQ ID NO:3, SEQ ID NO:4, SEQ ID NO:5, SEQ ID NO:6, SEQ ID NO:21, SEQ ID NO:22, SEQ ID NO:23, SEQ ID NO:24, SEQ ID NO:25, SEQ ID NO: 26, SEQ ID NO: 30, or SEQ ID NO:31.

Accordingly, with respect to liver and bone specific expression, provided are composite nucleic acid regulatory elements that comprise or consist of promoters and/or other nucleic acid elements, such as enhancers that promote liver expression, such as ApoE enhancers, Mic/BiKE elements or hAAT promoters. These may be present as single copies or with two or more copies in tandem. The nucleic acid regulatory element may also comprise, in addition to the one or more elements that promotes liver specific expression, one or more elements that promote bone specific expression, for example one or more copies of the Sp7/Osx or minSp7/Osx elements, which may be arranged as two or more copies in tandem. In certain embodiments, a promoter element is deleted for the initiation codon, and preferably the element that has the modified initiation codon is the promoter that is the element at the 3′ of the nucleic acid regulatory element, for example, closest to the transgene.

The recombinant expression cassettes provided herein comprise i) a composite nucleic acid regulatory element comprising a) one copy of ApoE or two copies of ApoE arranged in tandem, b) a copy or two copies arranged in tandem of Sp/Osx or minSp7/Osx; c) a copy or two copies of a hAAT promoter arranged in tandem comprising at least one copy of hAAT which is start-codon modified (ΔATG), ii) a transgene to which the composite regulatory element is operably linked. In some embodiments, the nucleic acid regulatory element comprises LBTP1 or LBTP2 of Table 1 (SEQ ID NO: 30 or 31). In and some embodiments, the composite nucleic acid regulatory element is operably linked to a transgene. The transgene may be any one of the genes or nucleic acids encoding the therapeutic proteins listed in, but not limited to, Tables 4A-4D. In certain embodiments, the transgene is a therapeutic antibody, including a full length antibody or antigen binding fragment thereof, such as, a Fab fragment.

The tandem and composite promoters described herein result in preferred transcription start sites within the promoter region. See for example, the results of Example 10 and Table 14. Thus, in certain embodiments, the constructs described herein have a tandem or composite nucleic acid regulatory sequence that comprises an hAAT promoter (particularly a modified start codon hAAT promoter) and has a transcription start site of TCTCC (SEQ ID NO: 43) (corresponding to nt 1541-1545 of LMTP6 SEQ ID NO: 6), which overlaps with the active TTS found in hAAT (nt 355-359 of SEQ ID NO: 11 or SEQ ID NO: 12) or GGTACAATGACTCCTTTCG (SEQ ID NO: 41), which corresponds to nucleotides 139-157 of SEQ ID NO: 11, or GGTACAGTGACTCCTTTCG (SEQ ID NO: 42), which corresponds to nucleotides 139-157 of SEQ ID NO: 12. In other embodiments, the constructs described herein have a tandem or composite regulatory sequence that comprises a CK8 promoter and has a transcription start site at TCATTCTACC (SEQ ID NO: 46), which corresponds to nucleotides 377-386 of SEQ ID NO: 16, particularly starting at the nucleotide corresponding to nucleotide 377 of SEQ ID NO: 16 or corresponding to nucleotide 1133 of SEQ ID NO: 6.

In an aspect of the invention, various regulatory elements and combinations of elements were utilized to design and generate nucleic acid expression cassettes, and are listed in Table 1.

TABLE 1
Promoter sequences

| | SEQ | |
|---|---|---|
| Name | ID NO: | Nucleic Acid Sequence |
| LSPX1 | 1 | aggttaatttttaaaaagcagtcaaaagtccaagtggcccttggcagca |
| | | tttactctctctgtttgctctggttaataatctcaggagcacaaacatt |
| | | ccagatccaggttaatttttaaaaagcagtcaaaagtccaagtggccct |
| | | tggcagcatttactctctctgtttgctctggttaataatctcaggagca |
| | | caaacattccagatccggcgcgccagggctggaagctacctttgtctag |
| | | aaggctcagaggcacacaggagtttctgggctcaccctgcccccttcca |
| | | acccctcagttcccatcctccagcagctgtttgtgtgctgcctctgaag |
| | | tccacactgaacaaacttcagcctactcatgtccctaaaatgggcaaac |
| | | attgcaagcagcaaacagcaaacacacagccctccctgcctgctgacct |
| | | tggagctggggcagaggtcagagacctctctgggcccatgccacctcca |
| | | acatccactcgaccccttggaatttcggtggagaggagcagaggttgtc |
| | | ctggcgtggtttaggtagtgtgagaggggtacccggggatcttgctacc |
| | | agtggaacagccactaaggattctgcagtgagagcagagggccagctaa |
| | | gtggtactctcccagagactgtctgactcacgccaccccctccaccttg |
| | | gacacaggacgctgtggtttctgagccaggtacaatgactcctttcggt |
| | | aagtgcagtggaagctgtacactgcccaggcaaagcgtccgggcagcgt |
| | | aggcgggcgactcagatcccagccagtggacttagcccctgtttgctcc |
| | | tccgataactggggtgaccttggttaatattcaccagcagcctcccccg |
| | | ttgcccctctggatccactgcttaaatacggacgaggacagggccctgt |
| | | ctcctcagcttcaggcaccaccactgacctgggacagt |
| LSPX2 | 2 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagaaggctcagaggcacac |
| | | aggagtttctgggctcaccctgcccccttccaacccctcagttcccatc |
| | | ctccagcagctgtttgtgtgctgcctctgaagtccacactgaacaaact |
| | | tcagcctactcatgtccctaaaatgggcaaacattgcaagcagcaaaca |
| | | gcaaacacacagccctccctgcctgctgaccttggagctggggcagagg |
| | | tcagagacctctctgggcccatgccacctccaacatccactcgacccct |
| | | tggaatttcggtggagaggagcagaggttgtcctggcgtggtttaggta |
| | | gtgtgagaggggtacccggggatcttgctaccagtggaacagccactaa |
| | | ggattctgcagtgagagcagagggccagctaagtggtactctcccagag |
| | | actgtctgactcacgccaccccctccaccttggacacaggacgctgtgg |
| | | tttctgagccaggtacaatgactcctttcggtaagtgcagtggaagctg |
| | | tacactgcccaggcaaagcgtccgggcagcgtaggcgggcgactcagat |
| | | cccagccagtggacttagcccctgtttgctcctccgataactggggtga |
| | | ccttggttaatattcaccagcagcctcccccgttgcccctctggatcca |
| | | ctgcttaaatacggacgaggacagggccctgtctcctcagcttcaggca |
| | | ccaccactgacctgggacagt |
| LTP1 | 3 | aggttaatttttaaaaagcagtcaaaagtccaagtggcccttggcagca |
| | | tttactctctctgtttgctctggttaataatctcaggagcacaaacatt |
| | | ccagatccaggttaatttttaaaaagcagtcaaaagtccaagtggccct |
| | | tggcagcatttactctctctgtttgctctggttaataatctcaggagca |
| | | caaacattccagatccggcgcgccagggctggaagctacctttgacatc |
| | | atttcctctgcgaatgcatgtataatttctacagaacctattagaaagg |
| | | atcacccagcctctgcttttgtacaactttcccttaaaaaactgccaat |
| | | tccactgctgtttggcccaatagtgagaactttttcctgctgcctcttg |
| | | gtgcttttgcctatggcccctattctgcctgctgaagacactcttgcca |
| | | gcatggacttaaacccctccagctctgacaatcctctttctcttttgtt |
| | | ttacatgaagggtctggcagccaaagcaatcactcaaagttcaaacctt |
| | | atcattttttgctttgttcctcttggccttggttttgtacatcagcttt |
| | | gaaaataccatcccagggttaatgctggggttaatttataactaagagt |
| | | gctctagttttgcaatacaggacatgctataaaaatggaaagatgttgc |
| | | tttctgagaggatcttgctaccagtggaacagccactaaggattctgca |
| | | gtgagagcagagggccagctaagtggtactctcccagagactgtctgac |
| | | tcacgccaccccctccaccttggacacaggacgctgtggtttctgagcc |
| | | aggtacagtgactcctttcggtaagtgcagtggaagctgtacactgccc |
| | | aggcaaagcgtccgggcagcgtaggcgggcgactcagatcccagccagt |
| | | ggacttagcccctgtttgctcctccgataactggggtgaccttggttaa |
| | | tattcaccagcagcctcccccgttgcccctctggatccactgcttaaat |
| | | acggacgaggacagggccctgtctcctcagcttcaggcaccaccactga |
| | | cctgggacagt |
| LTP2 | 4 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagagcccttaagctagcag |
| | | gttaatttttaaaaagcagtcaaaagtccaagtggcccttggcagcatt |
| | | tactctctctgtttgctctggttaataatctcaggagcacaaacattcc |
| | | agatccaggttaatttttaaaaagcagtcaaaagtccaagtggcccttg |
| | | gcagcatttactctctctgtttgctctggttaataatctcaggagcaca |
| | | aacattccagatccggcgcgccagggctggaagctacctttgacatcat |
| | | ttcctctgcgaatgcatgtataatttctacagaacctattagaaaggat |
| | | cacccagcctctgcttttgtacaactttcccttaaaaaactgccaattc |
| | | cactgctgtttggcccaatagtgagaactttttcctgctgcctcttggt |
| | | gcttttgcctatggcccctattctgcctgctgaagacactcttgccagc |
| | | atggacttaaacccctccagctctgacaatcctctttctcttttgtttt |
| | | acatgaagggtctggcagccaaagcaatcactcaaagttcaaaccttat |
| | | cattttttgctttgttcctcttggccttggttttgtacatcagctttga |
| | | aaataccatcccagggttaatgctggggttaatttataactaagagtgc |
| | | tctagttttgcaatacaggacatgctataaaaatggaaagatgttgctt |
| | | tctgagaggatcttgctaccagtggaacagccactaaggattctgcagt |
| | | gagagcagagggccagctaagtggtactctcccagagactgtctgactc |
| | | acgccaccccctccaccttggacacaggacgctgtggtttctgagccag |
| | | gtacagtgactcctttcggtaagtgcagtggaagctgtacactgcccag |
| | | gcaaagcgtccgggcagcgtaggcgggcgactcagatcccagccagtgg |
| | | acttagcccctgtttgctcctccgataactggggtgaccttggttaata |
| | | ttcaccagcagcctcccccgttgcccctctggatccactgcttaaatac |
| | | ggacgaggacagggccctgtctcctcagcttcaggcaccaccactgacc |
| | | tgggacagt |
| LTP3 | 5 | aggttaatttttaaaaagcagtcaaaagtccaagtggcccttggcagca |
| | | tttactctctctgtttgctctggttaataatctcaggagcacaaacatt |
| | | ccagatccaggttaatttttaaaaagcagtcaaaagtccaagtggccct |
| | | tggcagcatttactctctctgtttgctctggttaataatctcaggagca |
| | | caaacattccagatccggcgcgccagggctggaagctacctttgacatc |
| | | atttcctctgcgaatgcatgtataatttctacagaacctattagaaagg |
| | | atcacccagcctctgcttttgtacaactttcccttaaaaaactgccaat |
| | | tccactgctgtttggcccaatagtgagaactttttcctgctgcctcttg |
| | | gtgcttttgcctatggcccctattctgcctgctgaagacactcttgcca |
| | | gcatggacttaaacccctccagctctgacaatcctctttctcttttgtt |
| | | ttacatgaagggtctggcagccaaagcaatcactcaaagttcaaacctt |
| | | atcattttttgctttgttcctcttggccttggttttgtacatcagcttt |
| | | gaaaataccatcccagggttaatgctggggttaatttataactaagagt |
| | | gctctagttttgcaatacaggacatgctataaaaatggaaagatgttgc |
| | | tttctgagaggatcttgctaccagtggaacagccactaaggattctgca |
| | | gtgagagcagagggccagctaagtggtactctcccagagactgtctgac |
| | | tcacgccaccccctccaccttggacacaggacgctgtggtttctgagcc |
| | | aggtacagtgactcctttcggtaagtgcagtggaagctgtacactgccc |
| | | aggcaaagcgtccgggcagcgtaggcgggcgactcagatcccagccagt |
| | | ggacttagcccctgtttgctcctccgataactggggtgaccttggttaa |
| | | tattcaccagcagcctcccccgttgcccctctggatccactgcttaaat |
| | | acggacgaggacagggccctgtctcctcagcttcaggcaccaccactga |
| | | cctgggacagtaaaacaggtaagtccgctgtttgtgtgctgcctctgaa |
| | | gtccacactgaacaaacttcagcctactcatgtccctaaaatgggcaaa |
| | | cattgcaagcagcaaacagcaaacacacagccctccctgcctgctgacc |
| | | ttggagctggggcagaggtcagagacctctctggcctctactaaccatg |
| | | ttcatgttttctttttttttctacaggtcctgggtgacgaacag |
| LMTP6 | 6 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggccactacgggtttaggctgccc |
| | | atgtaaggaggcaaggcctggggacacccgagatgcctggttataatta |
| | | acccagacatgtggctgccccccccccccccaacacctgctgcctctaa |
| | | aaataaccctgtccctggtggatcccactacgggtttaggctgcccatg |
| | | taaggaggcaaggcctggggacacccgagatgcctggttataattaacc |
| | | cagacatgtggctgccccccccccccccaacacctgctgcctctaaaaa |
| | | taaccctgtccctggtggatcccactacgggtttaggctgcccatgtaa |
| | | ggaggcaaggcctggggacacccgagatgcctggttataattaacccag |
| | | acatgtggctgccccccccccccccaacacctgctgcctctaaaaataa |
| | | ccctgtccctggtggatcccctgcatgcgaagatcttcgaacaaggctg |
| | | tgggggactgagggcaggctgtaacaggcttgggggccagggcttatac |
| | | gtgcctgggactcccaaagtattactgttccatgttcccggcgaagggc |
| | | cagctgtcccccgccagctagactcagcacttagtttaggaaccagtga |
| | | gcaagtcagcccttggggcagcccatacaaggccatggggctgggcaag |
| | | ctgcacgcctgggtccggggtgggcacggtgcccgggcaacgagctgaa |
| | | agctcatctgctctcaggggcccctccctggggacagcccctcctggct |
| | | agtcacaccctgtaggctcctctatataacccaggggcacaggggctgc |
| | | cctcattctaccaccacctccacagcacagacagacactcaggagccag |
| | | ccagcgtcgagatcttgctaccagtggaacagccactaaggattctgca |
| | | gtgagagcagagggccagctaagtggtactctcccagagactgtctgac |
| | | tcacgccaccccctccaccttggacacaggacgctgtggtttctgagcc |
| | | aggtacagtgactcctttcggtaagtgcagtggaagctgtacactgccc |
| | | aggcaaagcgtccgggcagcgtaggcgggcgactcagatcccagccagt |
| | | ggacttagcccctgtttgctcctccgataactggggtgaccttggttaa |
| | | tattcaccagcagcctcccccgttgcccctctggatccactgcttaaat |
| | | acggacgaggacagggccctgtctcctcagcttcaggcaccaccactga |
| | | cctgggacagt |
| LMTP13 | 21 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggggtacccggggatcttgctacca |
| | | gtctagaggccgtccgccctcggcaccatcctcacgacacccaaatatg |
| | | gcgacgggtgaggaatggtggggagttatttttagagcggtgaggaagg |
| | | tgggcaggcagcaggtgttggcgctctaaaaataactcccgggagttat |
| | | ttttagagcggaggaatggtggacacccaaatatggcgacggttcctca |
| | | cccgtcgccatatttgggtgtccgccctcggccggggccgcattcctgg |
| | | gggccgggcggtgctcccgcccgcctcgataaaaggctccggggccggc |
| | | ggcggcccacgagctacccggaggagcgggaggcgccaagcgtgagtat |
| | | cgatcttgctaccagtggaacagccactaaggattctgcagtgagagca |
| | | gagggccagctaagtggtactctcccagagactgtctgactcacgccac |
| | | cccctccaccttggacacaggacgctgtggtttctgagccaggtacagt |
| | | gactcctttcggtaagtgcagtggaagctgtacactgcccaggcaaagc |
| | | gtccgggcagcgtaggcgggcgactcagatcccagccagtggacttagc |
| | | ccctgtttgctcctccgataactggggtgaccttggttaatattcacca |
| | | gcagcctcccccgttgcccctctggatccactgcttaaatacggacgag |
| | | gacagggccctgtctcctcagcttcaggcaccaccactgacctgggaca |
| | | gt |
| LMTP14 | 22 | gaatggtggacacccaaatatggcgacggttcctcacccgtcgccatat |
| | | ttgggtgtccgccctcggccggggccgcattcctgggggccgggcggtg |
| | | ctcccgcccgcctcgataaaaggctccggggccggcggcggcccacgag |
| | | ctacccggaggagcgggaggcgccaagcgatcttgctaccagtggaaca |
| | | gccactaaggattctgcagtgagagcagagggccagctaagtggtactc |
| | | tcccagagactgtctgactcacgccaccccctccaccttggacacagga |
| | | cgctgtggtttctgagccaggtacagtgactcctttcggtaagtgcagt |
| | | ggaagctgtacactgcccaggcaaagcgtccgggcagcgtaggcgggcg |
| | | actcagatcccagccagtggacttagcccctgtttgctcctccgataac |
| | | tggggtgaccttggttaatattcaccagcagcctcccccgttgcccctc |
| | | tggatccactgcttaaatacggacgaggacagggccctgtctcctcagc |
| | | ttcaggcaccaccactgacctgggacagt |
| LMTP15 | 23 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagagaatggtggacaccca |
| | | aatatggcgacggttcctcacccgtcgccatatttgggtgtccgccctc |
| | | ggccggggccgcattcctgggggccgggcggtgctcccgcccgcctcga |
| | | taaaaggctccggggccggcggcggcccacgagctacccggaggagcgg |
| | | gaggcgccaagcgatcttgctaccagtggaacagccactaaggattctg |
| | | cagtgagagcagagggccagctaagtggtactctcccagagactgtctg |
| | | actcacgccaccccctccaccttggacacaggacgctgtggtttctgag |
| | | ccaggtacagtgactcctttcggtaagtgcagtggaagctgtacactgc |
| | | ccaggcaaagcgtccgggcagcgtaggcgggcgactcagatcccagcca |
| | | gtggacttagcccctgtttgctcctccgataactggggtgaccttggtt |
| | | aatattcaccagcagcctcccccgttgcccctctggatccactgcttaa |
| | | atacggacgaggacagggccctgtctcctcagcttcaggcaccaccact |
| | | gacctgggacagt |
| LMTP18 | 24 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggccactacgggtttaggctgccc |
| | | atgtaaggaggcaaggcctggggacacccgagatgcctggttataatta |
| | | acccagacatgtggctgccccccccccccccaacacctgctgcctctaa |
| | | aaataaccctgtccctggtggatcccctgcatgcgaagatcttcgaaca |
| | | aggctgtgggggactgagggcaggctgtaacaggcttgggggccagggc |
| | | ttatacgtgcctgggactcccaaagtattactgttccatgttcccggcg |
| | | aagggccagctgtcccccgccagctagactcagcacttagtttaggaac |
| | | cagtgagcaagtcagcccttggggcagcccatacaaggccatggggctg |
| | | ggcaagctgcacgcctgggtccggggtgggcacggtgcccgggcaacga |
| | | gctgaaagctcatctgctctcaggggcccctccctggggacagcccctc |
| | | ctggctagtcacaccctgtaggctcctctatataacccaggggcacagg |
| | | ggctgccctcattctaccaccacctccacagcacagacagacactcagg |
| | | agccagccagcgtcgagatcttgctaccagtggaacagccactaaggat |
| | | tctgcagtgagagcagagggccagctaagtggtactctcccagagactg |
| | | tctgactcacgccaccccctccaccttggacacaggacgctgtggtttc |
| | | tgagccaggtacagtgactcctttcggtaagtgcagtggaagctgtaca |
| | | ctgcccaggcaaagcgtccgggcagcgtaggcgggcgactcagatccca |
| | | gccagtggacttagcccctgtttgctcctccgataactggggtgacctt |
| | | ggttaatattcaccagcagcctcccccgttgcccctctggatccactgc |
| | | ttaaatacggacgaggacagggccctgtctcctcagcttcaggcaccac |
| | | cactgacctgggacagt |
| LMTP19 | 25 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggccctgcatgcgaagatcttcga |
| | | acaaggctgtgggggactgagggcaggctgtaacaggcttgggggccag |
| | | ggcttatacgtgcctgggactcccaaagtattactgttccatgttcccg |
| | | gcgaagggccagctgtcccccgccagctagactcagcacttagtttagg |
| | | aaccagtgagcaagtcagcccttggggcagcccatacaaggccatgggg |
| | | ctgggcaagctgcacgcctgggtccggggtgggcacggtgcccgggcaa |
| | | cgagctgaaagctcatctgctctcaggggcccctccctggggacagccc |
| | | ctcctggctagtcacaccctgtaggctcctctatataacccaggggcac |
| | | aggggctgccctcattctaccaccacctccacagcacagacagacactc |
| | | aggagccagccagcgtcgagatcttgctaccagtggaacagccactaag |
| | | gattctgcagtgagagcagagggccagctaagtggtactctcccagaga |
| | | ctgtctgactcacgccaccccctccaccttggacacaggacgctgtggt |
| | | ttctgagccaggtacagtgactcctttcggtaagtgcagtggaagctgt |
| | | acactgcccaggcaaagcgtccgggcagcgtaggcgggcgactcagatc |
| | | ccagccagtggacttagcccctgtttgctcctccgataactggggtgac |
| | | cttggttaatattcaccagcagcctcccccgttgcccctctggatccac |
| | | tgcttaaatacggacgaggacagggccctgtctcctcagcttcaggcac |
| | | caccactgacctgggacagt |
| LMTP20 | 26 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggcccttcagattaaaaataactg |
| | | aggtaagggcctgggtaggggaggtggtgtgagacgctcctgtctctcc |
| | | tctatctgcccatcggccctttggggaggaggaatgtgcccaaggacta |
| | | aaaaaaggccatggagccagaggggcgagggcaacagacctttcatggg |
| | | caaaccttggggccctgctgaagctttggcccactacgggtttaggctg |
| | | cccatgtaaggaggcaaggcctggggacacccgagatgcctggttataa |
| | | ttaacccagacatgtggctgccccccccccccccaacacctgctgcctc |
| | | taaaaataaccctgtccctggtggatcccctgcatgcgaagatcttcga |
| | | acaaggctgtgggggactgagggcaggctgtaacaggcttgggggccag |
| | | ggcttatacgtgcctgggactcccaaagtattactgttccatgttcccg |
| | | gcgaagggccagctgtcccccgccagctagactcagcacttagtttagg |
| | | aaccagtgagcaagtcagcccttggggcagcccatacaaggccatgggg |
| | | ctgggcaagctgcacgcctgggtccggggtgggcacggtgcccgggcaa |
| | | cgagctgaaagctcatctgctctcaggggcccctccctggggacagccc |
| | | ctcctggctagtcacaccctgtaggctcctctatataacccaggggcac |
| | | aggggctgccctcattctaccaccacctccacagcacagacagacactc |
| | | aggagccagccagcgtcgagatcttgctaccagtggaacagccactaag |
| | | gattctgcagtgagagcagagggccagctaagtggtactctcccagaga |
| | | ctgtctgactcacgccaccccctccaccttggacacaggacgctgtggt |
| | | ttctgagccaggtacagtgactcctttcggtaagtgcagtggaagctgt |
| | | acactgcccaggcaaagcgtccgggcagcgtaggcgggcgactcagatc |
| | | ccagccagtggacttagcccctgtttgctcctccgataactggggtgac |
| | | cttggttaatattcaccagcagcctcccccgttgcccctctggatccac |
| | | tgcttaaatacggacgaggacagggccctgtctcctcagcttcaggcac |
| | | caccactgacctgggacagt |
| Alpha-Mic/Bik | 7 | aggttaatttttaaaaagcagtcaaaagtccaagtggcccttggcagca |
| Enhancer | | tttactctctctgtttgctctggttaataatctcaggagcacaaacatt |
| (Mic/BikE) | | cc |
| Tandem (2) | 8 | aggttaatttttaaaaagcagtcaaaagtccaagtggcccttggcagca |
| alpha-Mic/Bik | | tttactctctctgtttgctctggttaataatctcaggagcacaaacatt |
| Enhancers | | ccaggttaatttttaaaaagcagtcaaaagtccaagtggcccttggcag |
| (2 Mic/BikE) | | catttactctctctgtttgctctggttaataatctcaggagcacaaaca |
| | | ttcc |
| ApoE Hepatic | 9 | aggctcagaggcacacaggagtttctgggctcaccctgcccccttccaa |
| Control Region | | cccctcagttcccatcctccagcagctgtttgtgtgctgcctctgaagt |
| containing | | ccacactgaacaaacttcagcctactcatgtccctaaaatgggcaaaca |
| ApoE | | ttgcaagcagcaaacagcaaacacacagccctccctgcctgctgacctt |
| Enhancer | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggg |
| Tandem (2) | 20 | aggctcagaggcacacaggagtttctgggctcaccctgcccccttccaa |
| ApoE | | cccctcagttcccatcctccagcagctgtttgtgtgctgcctctgaagt |
| Enhancers | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggtctagaaggctcagaggcacac |
| | | aggagtttctgggctcaccctgcccccttccaacccctcagttcccatc |
| | | ctccagcagctgtttgtgtgctgcctctgaagtccacactgaacaaact |
| | | tcagcctactcatgtccctaaaatgggcaaacattgcaagcagcaaaca |
| | | gcaaacacacagccctccctgcctgctgaccttggagctggggcagagg |
| | | tcagagacctctctg |
| TBG Promoter | 10 | gggctggaagctacctttgacatcatttcctctgcgaatgcatgtataa |
| | | tttctacagaacctattagaaaggatcacccagcctctgcttttgtaca |
| | | actttcccttaaaaaactgccaattccactgctgtttggcccaatagtg |
| | | agaactttttcctgctgcctcttggtgcttttgcctatggcccctattc |
| | | tgcctgctgaagacactcttgccagcatggacttaaacccctccagctc |
| | | tgacaatcctctttctcttttgttttacatgaagggtctggcagccaaa |
| | | gcaatcactcaaagttcaaaccttatcattttttgctttgttcctcttg |
| | | gccttggttttgtacatcagctttgaaaataccatcccagggttaatgc |
| | | tggggttaatttataactaagagtgctctagttttgcaatacaggacat |
| | | gctataaaaatggaaagat |
| hAAT | 11 | gatcttgctaccagtggaacagccactaaggattctgcagtgagagcag |
| Promoter | | agggccagctaagtggtactctcccagagactgtctgactcacgccacc |
| | | ccctccaccttggacacaggacgctgtggtttctgagccaggtaca [symbol] |
| | | actcctttcggtaagtgcagtggaagctgtacactgcccaggcaaagcg |
| | | tccgggcagcgtaggcgggcgactcagatcccagccagtggacttagcc |
| | | cctgtttgctcctccgataactggggtgaccttggttaatattcaccag |
| | | cagcctcccccgttgcccctctggatccactgcttaaatacggacgagg |
| | | acagggccctgtctcctcagcttcaggcaccaccactgacctgggacag |
| | | t |
| hAAT(ΔATG) | 12 | gatcttgctaccagtggaacagccactaaggattctgcagtgagagcag |
| Promoter | | agggccagctaagtggtactctcccagagactgtctgactcacgccacc |
| | | ccctccaccttggacacaggacgctgtggtttctgagccaggtaca [symbol] |
| | | actcctttcggtaagtgcagtggaagctgtacactgcccaggcaaagcg |
| | | tccgggcagcgtaggcgggcgactcagatcccagccagtggacttagcc |
| | | cctgtttgctcctccgataactggggtgaccttggttaatattcaccag |
| | | cagcctcccccgttgcccctctggatccactgcttaaatacggacgagg |
| | | acagggccctgtctcctcagcttcaggcaccaccactgacctgggacag |
| | | t |
| ApoE.hAAT | 37 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagaggggtacccggggatcttgctacca |
| | | gtggaacagccactaaggattctgcagtgagagcagagggccagctaag |
| | | tggtactctcccagagactgtctgactcacgccaccccctccaccttgg |
| | | acacaggacgctgtggtttctgagccaggtacaatgactcctttcggta |
| | | agtgcagtggaagctgtacactgcccaggcaaagcgtccgggcagcgta |
| | | ggcgggcgactcagatcccagccagtggacttagcccctgtttgctcct |
| | | ccgataactggggtgaccttggttaatattcaccagcagcctcccccgt |
| | | tgcccctctggatccactgcttaaatacggacgaggacagggccctgtc |
| | | tcctcagcttcaggcaccaccactgacctgggacagt |
| Mck Enhancer | 13 | ccactacgggtttaggctgcccatgtaaggaggcaaggcctggggacac |
| (MckE) | | ccgagatgcctggttataattaacccagacatgtggctgcccccccccc |
| | | ccccaacacctgctgcctctaaaaataaccctgtccctggtggatc |
| Tandem (2) | 14 | ccactacgggtttaggctgcccatgtaaggaggcaaggcctggggacac |
| Mck | | ccgagatgcctggttataattaacccagacatgtggctgcccccccccc |
| Enhancers | | ccccaacacctgctgcctctaaaaataaccctgtccctggtggatccca |
| (2 MckE) | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatc |
| Tandem Mck | 15 | ccactacgggtttaggctgcccatgtaaggaggcaaggcctggggacac |
| (3) Enhancers | | ccgagatgcctggttataattaacccagacatgtggctgcccccccccc |
| (3 MckE) | | ccccaacacctgctgcctctaaaaataaccctgtccctggtggatccca |
| | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatcccacta |
| | | cgggtttaggctgcccatgtaaggaggcaaggcctggggacacccgaga |
| | | tgcctggttataattaacccagacatgtggctgccccccccccccccaa |
| | | cacctgctgcctctaaaaataaccctgtccctggtggatc |
| Myosin heavy | 27 | cccttcagattaaaaataactgaggtaagggcctgggtaggggaggtgg |
| chain enhancer | | tgtgagacgctcctgtctctcctctatctgcccatcggccctttgggga |
| (MhcE) | | ggaggaatgtgcccaaggactaaaaaaaggccatggagccagaggggcg |
| | | agggcaacagacctttcatgggcaaaccttggggccctgctgaagcttt |
| | | ggc |
| CK8 Promoter | 16 | ccctgcatgcgaagatcttcgaacaaggctgtgggggactgagggcagg |
| | | ctgtaacaggcttgggggccagggcttatacgtgcctgggactcccaaa |
| | | gtattactgttccatgttcccggcgaagggccagctgtcccccgccagc |
| | | tagactcagcacttagtttaggaaccagtgagcaagtcagcccttgggg |
| | | cagcccatacaaggccatggggctgggcaagctgcacgcctgggtccgg |
| | | ggtgggcacggtgcccgggcaacgagctgaaagctcatctgctctcagg |
| | | ggcccctccctggggacagcccctcctggctagtcacaccctgtaggct |
| | | cctctatataacccaggggcacaggggctgccctcattctaccaccacc |
| | | tccacagcacagacagacactcaggagccagccagcgtcga |
| SPc5.12 | 28 | ggccgtccgccctcggcaccatcctcacgacacccaaatatggcgacgg |
| | | gtgaggaatggtggggagttatttttagagcggtgaggaaggtgggcag |
| | | gcagcaggtgttggcgctctaaaaataactcccgggagttatttttaga |
| | | gcggaggaatggtggacacccaaatatggcgacggttcctcacccgtcg |
| | | ccatatttgggtgtccgccctcggccggggccgcattcctgggggccgg |
| | | gcggtgctcccgcccgcctcgataaaaggctccggggccggcggcggcc |
| | | cacgagctacccggaggagcgggaggcgccaagc |
| Minimal | 29 | gaatggtggacacccaaatatggcgacggttcctcacccgtcgccatat |
| SPc5.12 | | ttgggtgtccgccctcggccggggccgcattcctgggggccgggcggtg |
| | | ctcccgcccgcctcgataaaaggctccggggccggcggcggcccacgag |
| | | ctacccggaggagcgggaggcgccaag |
| CAG Promoter | 17 | gacattgattattgactagttattaatagtaatcaattacggggtcatt |
| | | agttcatagcccatatatggagttccgcgttacataacttacggtaaat |
| | | ggcccgcctggctgaccgcccaacgacccccgcccattgacgtcaataa |
| | | tgacgtatgttcccatagtaacgccaatagggactttccattgacgtca |
| | | atgggtggagtatttacggtaaactgcccacttggcagtacatcaagtg |
| | | tatcatatgccaagtacgccccctattgacgtcaatgacggtaaatggc |
| | | ccgcctggcattatgcccagtacatgaccttatgggactttcctacttg |
| | | gcagtacatctacgtattagtcatcgctattaccatggtcgaggtgagc |
| | | cccacgttctgcttcactctccccatctcccccccctccccacccccaa |
| | | ttttgtatttatttattttttaattattttgtgcagcgatgggggcggg |
| | | gggggggggggggcgcgcgccaggcggggcggggcggggcgaggggcgg |
| | | ggcggggcgaggcggagaggtgcggcggcagccaatcagagcggcgcgc |
| | | tccgaaagtttccttttatggcgaggcggcggcggcggcggccctataa |
| | | aaagcgaagcgcgcggcgggcgggagtcgctgcgcgctgccttcgcccc |
| | | gtgccccgctccgccgccgcctcgcgccgcccgccccggctctgactga |
| | | ccgcgttactcccacaggtgagcgggcgggacggcccttctcctccggg |
| | | ctgtaattagcgcttggtttaatgacggcttgtttcttttctgtggctg |
| | | cgtgaaagccttgaggggctccgggagggccctttgtgcggggggagcg |
| | | gctcggggggtgcgtgcgtgtgtgtgtgcgtggggagcgccgcgtgcgg |
| | | ctccgcgctgcccggcggctgtgagcgctgcgggcgcggcgcggggctt |
| | | tgtgcgctccgcagtgtgcgcgaggggagcgcggccgggggcggtgccc |
| | | cgcggtgcggggggggctgcgaggggaacaaaggctgcgtgcggggtgt |
| | | gtgcgtgggggggtgagcagggggtgtgggcgcgtcggtcgggctgcaa |
| | | ccccccctgcacccccctccccgagttgctgagcacggcccggcttcgg |
| | | gtgcggggctccgtacggggcgtggcgcggggctcgccgtgccgggcgg |
| | | ggggtggcggcaggtgggggtgccgggcggggcggggccgcctcgggcc |
| | | ggggagggctcgggggaggggcgcggcggcccccggagcgccggcggct |
| | | gtcgaggcgcggcgagccgcagccattgccttttatggtaatcgtgcga |
| | | gagggcgcagggacttcctttgtcccaaatctgtgcggagccgaaatct |
| | | gggaggcgccgccgcaccccctctagcgggcgcggggcgaagcggtgcg |
| | | gcgccggcaggaaggaaatgggcggggagggccttcgtgcgtcgccgcg |
| | | ccgccgtccccttctccctctccagcctcggggctgtccgcggggggac |
| | | ggctgccttcgggggggacggggcagggcggggttcggcttctggcgtg |
| | | tgaccggcggctctagagcctctgctaaccatgttcatgccttcttctt |
| | | tttcctacag |
| LBTP1 | 30 | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | AGGCTCAGAGGCACACAGGAGTTTCTGGGCTCACCCTGCCCCCTTCCAA |
| | | CCCCTCAGTTCCCATCCTCCAGCAGCTGTTTGTGTGCTGCCTCTGAAGT |
| | | CCACACTGAACAAACTTCAGCCTACTCATGTCCCTAAAATGGGCAAACA |
| | | TTGCAAGCAGCAAACAGCAAACACACAGCCCTCCCTGCCTGCTGACCTT |
| | | GGAGCTGGGGCAGAGGTCAGAGACCTCTCTGGGCCCATGCCACCTCCAA |
| | | CATCCACTCGACCCCTTGGAATTTCGGTGGAGAGGAGCAGAGGTTGTCC |
| | | TGGCGTGGTTTAGGTAGTGTGAGAGGGCGCGGCAGCAGCGGCGGCAGCC |
| | | TCGGTGGTAGCAGCAGCAGCAGCAGCAGCAGCAGCAGCAGCAGCAGCAG |
| | | CAGCAGCAACAGAAGCTGCCGCGCCGCTGAGTAGCAGCAAGGACTCCGA |
| | | GTCAAGAGTAGGATTGTAGGATTGGATCTGAGTGGGAACAAGAGTGAGC |
| | | TGGCCTGAGAGAGGAGCAGATGCCTCCCAGCGCCCTCAGGCCACCCATT |
| | | GCCAGTAATCTTCAAGCCAGACCTCTTGAGAGGAGACGGGACAGCCAAC |
| | | CCTAGCCTACCCAGGTACAGACACTGGGCAGTTCTGGGGGACTGCCCAC |
| | | AGATGCCTATTGGATTCCTGGGGTATGTAGGACTCCCGGGTCTACCAGC |
| | | CCTTTTCACCTTTCCCCATAGCACCCCCAAGGAAGCTCTGACAACTTGC |
| | | CCATATTCCTGTTTCCCACCCGTCCCCTGGGCACCCCCTTTTCTTCTCT |
| | | CCCTCCCAGATCCCTTCTTTGGGGAGCTCAGCAAATGGAGCAGGAAATT |
| | | TGGACCCTCTGCCTCCCTCTCTCGCCTTCCTCATTGGATCCGGAGTCTT |
| | | CTCCGCTGGGAAAGCTGTAATTAGAGGGTGGATCCCTACAGACAGAGAG |
| | | CAGCCCCCCCACCCCCACCCCCCAGTCCCTTCTAACTTTAGATCTCTTC |
| | | TCTCCCATTCTCCCATTCTCCCTCCCTCTCCCTTCTCCCTCTCCCACTG |
| | | GCTCCTCGGTTCTCTCCATCTGCCTGACTCCTTGGGACCCGGTCCCCAG |
| | | ATCTTGCTACCAGTGGAACAGCCACTAAGGATTCTGCAGTGAGAGCAGA |
| | | GGGCCAGCTAAGTGGTACTCTCCCAGAGACTGTCTGACTCACGCCACCC |
| | | CCTCCACCTTGGACACAGGACGCTGTGGTTTCTGAGCCAGGTACAGTGA |
| | | CTCCTTTCGGTAAGTGCAGTGGAAGCTGTACACTGCCCAGGCAAAGCGT |
| | | CCGGGCAGCGTAGGCGGGCGACTCAGATCCCAGCCAGTGGACTTAGCCC |
| | | CTGTTTGCTCCTCCGATAACTGGGGTGACCTTGGTTAATATTCACCAGC |
| | | AGCCTCCCCCGTTGCCCCTCTGGATCCACTGCTTAAATACGGACGAGGA |
| | | CAGGGCCCTGTCTCCTCAGCTTCAGGCACCACCACTGACCTGGGACAGT |
| LBTP2 | 31 | aggctcagaggcacacaggagtttctgggctcaccctgcccccttccaa |
| | | cccctcagttcccatcctccagcagctgtttgtgtgctgcctctgaagt |
| | | ccacactgaacaaacttcagcctactcatgtccctaaaatgggcaaaca |
| | | ttgcaagcagcaaacagcaaacacacagccctccctgcctgctgacctt |
| | | ggagctggggcagaggtcagagacctctctgggcccatgccacctccaa |
| | | catccactcgaccccttggaatttcggtggagaggagcagaggttgtcc |
| | | tggcgtggtttaggtagtgtgagagggcacacatacacgaacacacata |
| | | catatacatacattcacatatatgcacacacatacacatatacacgcat |
| | | acacgtacacacaaatgcacactcacacatgcacacacatacataatat |
| | | acacactctcacacatgcacatacacacatacatacacatacatgtgca |
| | | tgcacacacacaaatacacatgcatacatccacattcacacagatgcag |
| | | acacaaatgcacacacacacacacacacacacacacacacacacacaca |
| | | cacgcacactgccaccctgaactagtggtggctaaatgaacaataagtc |
| | | tccatcaccagcttggggggaggtaggtggtagtgtaggtgcccccatt |
| | | gtgtgatcatgttcattgtatgagtttgtctgtgttcattcatcatagt |
| | | gacagtccccatgtgggtagcagagagtacgtgtgcatgcatcatctcc |
| | | gtgtttgctcatgagtgtgtatgtcagtgtgttccagtctttctgtgtg |
| | | agtgtcgtccccaatcccccatccccccccccagatctctaattagtgg |
| | | tttggggtttgttccttttccctcctgttcctttcctcagcagcgcggc |
| | | agcagcggcggcagcctcggtggtagcagcagcagcagcagcagcagca |
| | | gcagcagcagcagcagcagcagcaacagaagctgccgcgccgctgagta |
| | | gcagcaaggactccgagtcaagagtaggattgtaggattggatctgagt |
| | | gggaacaagagtgagctggcctgagagaggagcagatgcctcccagcgc |
| | | cctcaggccacccattgccagtaatcttcaagccagacctcttgagagg |
| | | agacgggacagccaaccctagcctacccaggtacagacactgggcagtt |
| | | ctgggggactgcccacagatgcctattggattcctggggtatgtaggac |
| | | tcccgggtctaccagcccttttcacctttccccatagcacccccaagga |
| | | agctctgacaacttgcccatattcctgtttcccacccgtcccctgggca |
| | | cccccttttcttctctccctcccagatcccttctttggggagctcagca |
| | | aatggagcaggaaatttggaccctctgcctccctctctcgccttcctca |
| | | ttggatccggagtcttctccgctgggaaagctgtaattagagggtggat |
| | | ccctacagacagagagcagcccccccacccccaccccccagtcccttct |
| | | aactttagatctcttctctcccattctcccattctccctccctctccct |
| | | tctccctctcccactggctcctcggttctctccatctgcctgactcctt |
| | | gggacccggtccccagatcttgctaccagtggaacagccactaaggatt |
| | | ctgcagtgagagcagagggccagctaagtggtactctcccagagactgt |
| | | ctgactcacgccaccccctccaccttggacacaggacgctgtggtttct |
| | | gagccaggtacagtgactcctttcggtaagtgcagtggaagctgtacac |
| | | tgcccaggcaaagcgtccgggcagcgtaggcgggcgactcagatcccag |
| | | ccagtggacttagcccctgtttgctcctccgataactggggtgaccttg |
| | | gttaatattcaccagcagcctcccccgttgcccctctggatccactgct |
| | | taaatacggacgaggacagggccctgtctcctcagcttcaggcaccacc |
| | | actgacctgggacagt |
| SP7 | 32 | cacacatacacgaacacacatacatatacatacattcacatatatgcac |
| | | acacatacacatatacacgcatacacgtacacacaaatgcacactcaca |
| | | catgcacacacatacataatatacacactctcacacatgcacatacaca |
| | | catacatacacatacatgtgcatgcacacacacaaatacacatgcatac |
| | | atccacattcacacagatgcagacacaaatgcacacacacacacacaca |
| | | cacacacacacacacacacacacacgcacactgccaccctgaactagtg |
| | | gtggctaaatgaacaataagtctccatcaccagcttggggggaggtagg |
| | | tggtagtgtaggtgcccccattgtgtgatcatgttcattgtatgagttt |
| | | gtctgtgttcattcatcatagtgacagtccccatgtgggtagcagagag |
| | | tacgtgtgcatgcatcatctccgtgtttgctcatgagtgtgtatgtcag |
| | | tgtgttccagtctttctgtgtgagtgtcgtccccaatcccccatccccc |
| | | cccccagatctctaattagtggtttggggtttgttccttttccctcctg |
| | | ttcctttcctcagcagcgcggcagcagcggcggcagcctcggtggtagc |
| | | agcagcagcagcagcagcagcagcagcagcagcagcagcagcagcaaca |
| | | gaagctgccgcgccgctgagtagcagcaaggactccgagtcaagagtag |
| | | gattgtaggattggatctgagtgggaacaagagtgagctggcctgagag |
| | | aggagcagatgcctcccagcgccctcaggccacccattgccagtaatct |
| | | tcaagccagacctcttgagaggagacgggacagccaaccctagcctacc |
| | | caggtacagacactgggcagttctgggggactgcccacagatgcctatt |
| | | ggattcctggggtatgtaggactcccgggtctaccagcccttttcacct |
| | | ttccccatagcacccccaaggaagctctgacaacttgcccatattcctg |
| | | tttcccacccgtcccctgggcacccccttttcttctctccctcccagat |
| | | cccttctttggggagctcagcaaatggagcaggaaatttggaccctctg |
| | | cctccctctctcgccttcctcattggatccggagtcttctccgctggga |
| | | aagctgtaattagagggtggatccctacagacagagagcagccccccca |
| | | cccccaccccccagtcccttctaactttagatctcttctctcccattct |
| | | cccattctccctccctctcccttctccctctcccactggctcctcggtt |
| | | ctctccatctgcctgactccttgggacccggtcccca |
| Minimal SP7 | 33 | cgcggcagcagcggcggcagcctcggtggtagcagcagcagcagcagca |
| | | gcagcagcagcagcagcagcagcagcagcaacagaagctgccgcgccgc |
| | | tgagtagcagcaaggactccgagtcaagagtaggattgtaggattggat |
| | | ctgagtgggaacaagagtgagctggcctgagagaggagcagatgcctcc |
| | | cagcgccctcaggccacccattgccagtaatcttcaagccagacctctt |
| | | gagaggagacgggacagccaaccctagcctacccaggtacagacactgg |
| | | gcagttctgggggactgcccacagatgcctattggattcctggggtatg |
| | | taggactcccgggtctaccagcccttttcacctttccccatagcacccc |
| | | caaggaagctctgacaacttgcccatattcctgtttcccacccgtcccc |
| | | tgggcacccccttttcttctctccctcccagatcccttctttggggagc |
| | | tcagcaaatggagcaggaaatttggaccctctgcctccctctctcgcct |
| | | tcctcattggatccggagtcttctccgctgggaaagctgtaattagagg |
| | | gtggatccctacagacagagagcagcccccccacccccaccccccagtc |
| | | ccttctaactttagatctcttctctcccattctcccattctccctccct |
| | | ctcccttctccctctcccactggctcctcggttctctccatctgcctga |
| | | ctccttgggacccggtcccca |

5.2.1 Enhancers

The present inventors have surprisingly discovered multiple enhancers are amenable to tandem positioning while operably linked to one or more promoters. These enhancers when arranged in tandem and operably linked to promoters and a transgene promote tissue specific expression of the transgenes.

Accordingly, provided are ApoE enhancers, particularly an ApopE Hepatic Control Region containing an ApoE Enhancer, as in SEQ ID NO: 9.

Accordingly, provided are alpha-1-microglobulin/bikunin (alpha-Mic/Bik) enhancers either as a single copy or two copies arranged in tandem (SEQ ID NOs: 7 and 8, respectively in Table 1). The enhancer activity of alpha-Mic/Bik was found to be restricted to liver cells (Rouet, P. et al. 1992 J Biol Chem. October 15; 267(29):20765-73).

Accordingly, provided are muscle-specific enhancer (MckE) nucleic acid elements as a single copy, or two or three copies arranged in tandem (SEQ ID NOs: 13, 14 and 15, respectively, in Table 1) from the mouse muscle creatine kinase gene (Jaynes, J. B., Johnson, J. E., Buskin, J. N., Gartside, C. L., and Hauschka, S. D. The muscle creatine kinase gene is regulated by multiple upstream elements, including the muscle-specific enhancer. Mol. Cell. Biol., 8: 62-70, 1988; and GenBank Accn. No. AF188002.1). The 206-bp fragment from this region acts as a skeletal muscle enhancer and confers orientation-dependent activity in myocardiocytes. A 110-bp enhancer subfragment of this sequence confers high-level expression in skeletal myocytes but is inactive in myocardiocytes (Amacher, et al. 1993 Molecular and Cellular Biology 13(5):2753-64).

Also provided are Myosin heavy chain enhancer (MhcE) nucleic acids (SEQ ID NO: 27, in Table 1) placed in tandem with additional regulatory elements. Myosin is the most abundant protein in muscle, which is the most abundant tissue in the body. Enhancement of muscle production of transgene, including skeletal and cardiac muscle expression, would greatly benefit the biotherapeutic effect of many transgenes.

Other enhancers are well known to the skilled person in the art.

5.2.2 Promoters

Another aspect of the present invention relates to nucleic acid expression cassettes comprising chimeric regulatory elements designed to confer or enhance liver-specific expression, muscle-specific expression (including skeletal or cardiac muscle specific expression) or bone-specific expression. The invention involves engineering regulatory elements in tandem, including promoter elements, enhancer elements, and optionally introns. Examples include but are not limited to TBG promoters, hAAT promoters, CK8 promoters, and SPc5-12 promoters.

The unique combinations of promoter and enhancer sequences provided herein improve transgene expression while maintaining tissue specificity. The novel regulatory element nucleic acids were generated using a method to improve transgene expression from tandem promoters (i.e. two promoter sequences driving expression of the same transgene) by depleting the 3′ promoter sequence of potential ‘ATG’ initiation sites. This approach was employed to improve transgene expression from tandem tissue-specific promoter cassettes (such as those targeting the liver) as well as promoter cassettes to achieve dual expression in two separate tissue populations (such as liver and skeletal muscle, and in certain embodiments cardiac muscle, and liver and bone). Ultimately, these designs aim to improve the therapeutic efficacy of gene transfer by providing more robust levels of transgene expression, improved stability/persistence, and induction of immune tolerance to the transgene product. In certain aspects the hAAT promoter with the start codon deleted (ΔATG) is used in an expression cassette provided herein.

The CAG promoter (SEQ ID NO: 17) refers to a chimeric promoter constructed from the following sequences: the cytomegalovirus (CMV) early enhancer element (C), the chicken beta-actin promoter (the first exon and the first intron of chicken beta-actin gene) (A), and the splice acceptor of the rabbit beta-globin gene (G). The CAG promoter is frequently utilized in the art to drive high levels of expression in mammalian cells, and is non-preferential with respect to tissue specificity, therefore is typically utilized as a universal promoter.

Also provided are bone specific promoters that may be arranged in combination with liver specific expression elements. For example the Sp7/Osx promoter (SEQ ID NO: 32) or minimal Sp7/Osx promoter (SEQ ID NO: 33) fragment (Lu, X., et al. JBC 281, 6297-6306, Jan. 12, 2006, herein incorporated by reference in its entirety) promotes bone specific expression and may be included as either a single copy or two or more copies arranged in tandem in the gene cassettes provided herein.

Also provided is the muscle-specific synthetic promoter c5-12 (Li, X. et al. Nature Biotechnology Vol. 17, pp. 241-245, MARCH 1999), known as the SPc5-12 promoter. When arranged in tandem with enhancers or other promoters and operably linked to a transgene, the SPc5-12 promoter drives muscle-specific expression of the transgenes. In some embodiments, the muscle-specific promoter is a SPc5-12 promoter (SEQ ID NO: 28).

In order to further reduce the length of a vector, regulatory elements can be a reduced or shortened version (referred to herein as a “minimal promoter”) of any one of the promoters described herein. A minimal promoter comprises at least the transcriptionally active domain of the full-length version and is therefore still capable of driving expression. For example, in some embodiments, the transcriptionally active domain of a muscle-specific promoter, e.g., a minimal SPc5-12 promoter (e.g., SEQ ID NO: 29), can be placed in tandem with additional regulatory elements and be operably linked to a therapeutic protein transgene.

5.2.3 Introns

Another aspect of the present invention relates to nucleic acid expression cassettes comprising an intron within the regulatory cassette. In some embodiments, the intron nucleic acid is a chimeric intron derived from human β-globin and Ig heavy chain (also known as (3-globin splice donor/immunoglobulin heavy chain splice acceptor intron, or β-globin/IgG chimeric intron, Reed, R., et al. Genes and Development, 1989). Use of an intron may further induce efficient splicing in eukaryotic cells. Although use of an intron may not indicate increases in expression to an already strong promoter, the presence of an intron may increase the expression level of transgene and can also increase the duration of expression in vivo.

In some embodiments, the intron is a VH4 intron. The VH4 intron nucleic acid can comprise SEQ ID NO: 19 as shown in Table 2 below. The VH4 intron 5′ of the coding sequence may enhance proper splicing and, thus, transgene expression. Accordingly, in some embodiments, an intron is coupled to the 5′ end of a transgene sequence. In other embodiments, the intron is less than 100 nucleotides in length.

TABLE 2
Nucleotide sequences for different introns

| | SEQ | |
|---|---|---|
| Structure | ID | Sequence |
| Chimeric intron | 18 | GTAAGTATCAAGGTTACAAGACAGGTTTAA |
| (β-globin/Ig | | GGAGACCAATAGAAACTGGGCTTGTCGAGA |
| Intron) | | CAGAGAAGACTCTTGCGTTTCTGATAGGCA |
| | | CCTATTGGTCTTACTGACATCCACTTTGCC |
| | | TTTCTCTCCACAG |
| VH4 intron | 19 | GTGAGTATCTCAGGGATCCAGACATGGGGA |
| | | TATGGGAGGTGCCTCTGATCCCAGGGCTCA |
| | | CTGTGGGTCTCTCTGTTCACAG |
| SV40 intron | 34 | GTAAGTTTAGTCTTTTTGTCTTTTATTTCA |
| | | GGTCCCGGATCCGGTGGTGGTGCAAATCAA |
| | | AGAACTGCTCCTCAGTGGATGTTGCCTTTA |
| | | CTTCTAG |

In other embodiments, the intron is a chimeric intron derived from human β-globin and Ig heavy chain (also known as β-globin splice donor/immunoglobulin heavy chain splice acceptor intron, or β-globin/IgG chimeric intron) (Table 3, SEQ ID NO: 18). Other introns well known to the skilled person may be employed, such as the chicken β-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron (Table 3, SEQ ID NO: 34).

Other introns well known to the skilled person may be employed.

### 5.2.4 Other Regulatory Elements

5.2.4.1 polyA

Another aspect of the present disclosure relates to expression cassettes comprising a polyadenylation (polyA) site downstream of the coding region of the transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene (SEQ ID NO: 36), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57. In one embodiment, the polyA signal comprises SEQ ID NO: 36 as shown in Table 3.

TABLE 3
Nucleotide Sequence of the PolyA Signal

| | SEQ ID | |
|---|---|---|
| Structure | NO: | Sequence |
| β-globin PolyA | 35 | ataaaggaaatttattttcattgcaa |
| signal | | tagtgtgttggaattttttgtgtctc |
| | | tca |
| Rabbit β-globin | 36 | gatctttttccctctgccaaaaatta |
| polyA | | tggggacatcatgaagccccttgagc |
| | | atctgacttctggctaataaaggaaa |
| | | tttattttcattgcaatagtgtgttg |
| | | gaattttttgtgtctctcactcg |

### 5.3. Vectors for Gene Delivery

Another aspect of the present invention relates to the genetic engineering of tandem nucleic acid regulatory elements and incorporating these nucleic acid sequences in a vector expression system. In one embodiment, the vector is a viral vector, including but not limited to recombinant adeno-associated viral (rAAV) vectors (e.g. Gao G., et al 2003 Proc. Natl. Acad. Sci. U.S.A. 100 (10): 6081-6086), lentiviral vectors (e.g. Matrai, J, et al. 2011, Hepatology 53, 1696-707), retroviral vectors (e.g. Axelrod, J H, et al. 1990. Proc Natl Acad Sci USA; 87, 5173-7), adenoviral vectors (e.g. Brown et al., 2004 Blood 103, 804-10), herpes-simplex viral vectors (Marconi, P. et al. Proc Natl Acad Sci USA. 1996 93(21): 11319-11320; Baez, M V, et al. Chapter 19—Using Herpes Simplex Virus Type 1-Based Amplicon Vectors for Neuroscience Research and Gene Therapy of Neurologic Diseases, Ed.: Robert T. Gerlai, Molecular-Genetic and Statistical Techniques for Behavioral and Neural Research, Academic Press, 2018:Pages 445-477), and retrotransposon-based vector systems (e.g. Soifer, 2004, Current Gene Therapy 4(4):373-384). In another embodiment, the vector is a non-viral vector. rAAV vectors have limited packaging capacity of the vector particles (i.e. approximately 4.7 kb), constraining the size of the transgene expression cassette to obtain functional vectors (Jiang et al., 2006 Blood. 108:107-15). The length of the transgene and the length of the regulatory nucleic acid sequences comprising tandem enhancer(s) and promoter(s) are taken into consideration when selecting a regulatory region suitable for a particular transgene and target tissue.

Another aspect of the present invention relates to a viral vector comprising an expression cassette comprising a nucleic acid regulatory element LSPX1, LSPX2, LTP1, LTP2, or LTP3 of Table 1, operably linked to a transgene. In some embodiments, the expression cassette comprises a nucleic acid regulatory element comprising the nucleic acid sequence of SEQ ID NO: 1, SEQ ID NO: 2, SEQ ID NO: 3, SEQ ID NO: 4, or SEQ ID NO: 5, or a sequence that is 99%, 95%, 90%, 85% or 80% identical to SEQ ID NO: 1, SEQ ID NO: 2, SEQ ID NO: 3, SEQ ID NO: 4, or SEQ ID NO: 5 and enhances expression of the transgene in liver.

Another aspect of the present invention relates to a recombinant vector comprising an expression cassette comprising a nucleic acid regulatory element LMTP6, LMTP13, LMTP14, LMTP15, LMTP18, LMTP19, or LMTP20 of Table 1, operably linked to a transgene. In some embodiments, the expression cassette comprises a nucleic acid regulatory element comprising a nucleic acid sequence SEQ ID NO: 6, SEQ ID NO: 21, SEQ ID NO: 22, SEQ ID NO: 23, SEQ ID NO: 24, or SEQ ID NO: 25 or SEQ ID NO: 26 or a sequence that is 99%, 95%, 90%, 85% or 80% identical to SEQ ID NO: 6, SEQ ID NO: 21, SEQ ID NO: 22, SEQ ID NO: 23, SEQ ID NO: 24, or SEQ ID NO: 25 or SEQ ID NO: 26 and enhances expression in liver and muscle (skeletal and/or cardiac muscle) of the transgene.

Another aspect of the present invention relates to a recombinant vector comprising an expression cassette comprising a nucleic acid regulatory element LBTP1 (SEQ ID NO: 30) or LBTP2 (SEQ ID NO: 31) of Table 1, operably linked to a transgene. In some embodiments, the expression cassette comprises a nucleic acid regulatory element comprising a nucleic acid sequence of SEQ ID NO: 30 or SEQ ID NO: 31 or a sequence that is 99%, 95%, 90%, 85% or 80% identical to SEQ ID NO: 30 or SEQ ID NO: 31 and enhances expression in liver and bone of the transgene.

In another aspect, the expression cassettes are suitable for packaging in an AAV capsid, as such the cassette comprises (1) AAV inverted terminal repeats (ITRs) flank the expression cassette; (2) regulatory control elements, a) promoter/enhancers, such as any one of LSPX1, LSPX2, LTP1, LTP2, LTP3, LMTP6, LMTP13, LMTP14, LMTP15, LMTP18, LMTP19, LMTP20, LBTP1, or LBTP2 as in Table 1, b) a poly A signal, and c) optionally an intron; and (3) a transgene providing (e.g., coding for) one or more RNA or protein products of interest.

In certain embodiments, the transgene is from Tables 4A-4D. In embodiments for expressing an intact or substantially intact mAb, the constructs described herein comprise the following components: (1) AAV2 inverted terminal repeats that flank the expression cassette; (2) regulatory control elements, a) promoter/enhancers, such as any one of LSPX1, LSPX2, LTP1, LTP2, LTP3, LMTP6, LMTP13, LMTP14, LMTP15, LMTP18, LMTP19, LMTP20, LBTP1, or LBTP2 as in Table 1, b) a poly A signal, and c) optionally an intron; and (3) nucleic acid sequences coding for the heavy chain Fab of an anti-Aβ (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651,), anti-ALK1 (e.g., ascrinvacumab), anti-05 (e.g., tesidolumab, ravulizumab, and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof; and the light chain of an anti-Aβ (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651,), anti-ALK1 (e.g., ascrinvacumab), anti-05 (e.g., tesidolumab, ravulizumab, and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); wherein the heavy chain (Fab and Fc region) and the light chain are separated by a self-cleaving furin (F)/F2A or furin (F)/T2A or flexible linker, ensuring expression of equal amounts of the heavy and the light chain polypeptides.

In the various embodiments, the target tissue may be neural tissue, bone, kidney, liver, muscle, heart spleen, lung or endothelial tissue, or a particular receptor or tumor, and the regulatory agent is derived from a heterologous protein or domain that specifically recognizes and/or binds that tissue, particularly liver and muscle or liver and bone. The transgenes expressed in liver and muscle or liver and bone are considered systemic expression, since enhanced delivery of liver-expressed protein may be sufficient to cross into other tissues including crossing the blood brain barrier to the CNS and delivering therapeutics for treating neurological disorders or neurological symptoms of a systemic disorder.

In some embodiments, LBTP1 and LBTP2 promoters are particularly useful with any transgene in a gene therapy vector where it is desirous to confer expression of the gene therapy vector specifically in bone cells (such as osteoblasts) and liver cells (hepatocytes). The gene therapies thereof may be used for treatment of bone diseases and disorders and/or symptoms of any systemic disorder affecting the bone. For example, a gene therapy vector comprising a LBTP1 or LBTP2 promoter may be effective to ameliorate the bone-deforming symptoms of a systemic disorder, such as a lysosomal storage disease with skeletal involvement.

5.3.1 AAV

Another aspect of the present invention relates to expression cassettes suitable for packaging in an AAV capsid, as such the cassette comprises (1) AAV inverted terminal repeats (ITRs) flank the expression cassette; (2) regulatory control elements, consisting essentially of one or more enhancers and one or more promoters, particularly one of the muscle-liver specific or muscle-bone specific nucleic acid regulatory elements provided herein, d) a poly A signal, and e) optionally, an intron; and (3) a transgene providing (e.g., coding for) one or more RNA or protein products of interest.

The provided nucleic acids and methods are suitable for use in the production of any isolated recombinant AAV particles, in the production of a composition comprising any isolated recombinant AAV particles, or in the method for treating a disease or disorder in a subject in need thereof comprising the administration of any isolated recombinant AAV particles. As such, the rAAV may be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAV-PHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689, (see, e.g., SEQ ID NOs: 5-38 in '689 publication) WO2009/104964 (see, e.g., SEQ ID NOs: 1-5, 7, 9, 20, 22, 24 and 31 in '964 publication), WO2010/127097 (see, e.g., SEQ ID NOs: 5-38 in '097 publication), and WO 2015/191508 (see, e.g., SEQ ID NOs: 80-294 in '508 publication), and U.S. Appl. Publ. No. 20150023924 (see, e.g., SEQ ID NOs: 1, 5-10 in '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689 (see, e.g., SEQ ID NOs: 5-38 in '689 publication) WO2009/104964 (see, e.g., SEQ ID NOs: 1-5, 7, 9, 20, 22, 24 and 31 in '964 publication), WO 2010/127097 (see, e.g., SEQ ID NOs: 5-38 in '097 publication), and WO 2015/191508 (see, e.g., SEQ ID NOs: 80-294 of in '508 publication), and U.S. Appl. Publ. No. 20150023924 (see, e.g., SEQ ID NOs: 1, 5-10 in '924 publication).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for used in the production of recombinant AAV encoding a transgene. In some embodiments, provided herein are rAAV viral vectors encoding an anti-VEGF Fab. In some embodiments, provided herein are rAAV8-based viral vectors encoding an anti-VEGF Fab. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP. In some embodiments, provided herein are rAAV viral vectors encoding anti-kallikrein (anti-pKal) antibody. In some embodiments, provided herein are rAAV8-based or rAAV9-based viral vectors encoding a pKal antibody Fab or full-length antibody.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, rAAV particles comprise a capsid protein from an AAV capsid serotype selected from AAV-8 or AAV-9. In some embodiments, the rAAV particles have an AAV capsid serotype of AAV-1 or a derivative, modification, or pseudotype thereof. In some embodiments, the rAAV particles have an AAV capsid serotype of AAV-4 or a derivative, modification, or pseudotype thereof. In some embodiments, the rAAV particles have an AAV capsid serotype of AAV-5 or a derivative, modification, or pseudotype thereof. In some embodiments, the rAAV particles have an AAV capsid serotype of AAV-8 or a derivative, modification, or pseudotype thereof. In some embodiments, the rAAV particles have an AAV capsid serotype of AAV-9 or a derivative, modification, or pseudotype thereof.

In some embodiments, rAAV particles comprise a capsid protein that is a derivative, modification, or pseudotype of AAV-8 or AAV-9 capsid protein. In some embodiments, rAAV particles comprise a capsid protein that has an AAV-8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV-8 capsid protein.

In some embodiments, rAAV particles comprise a capsid protein that is a derivative, modification, or pseudotype of AAV-9 capsid protein. In some embodiments, rAAV particles in the clarified feed comprise a capsid protein that has an AAV-8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV-9 capsid protein.

In additional embodiments, rAAV particles comprise a mosaic capsid. Mosaic AAV particles are composed of a mixture of viral capsid proteins from different serotypes of AAV. In some embodiments, rAAV particles comprise a mosaic capsid containing capsid proteins of a serotype selected from AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles comprise a mosaic capsid containing capsid proteins of a serotype selected from AAV-1, AAV-2, AAV-5, AAV-6, AAV-7, AAV-8, AAV-9, AAV-10, AAVrh.8, and AAVrh.10. In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV-1, AAV-3, AAV-4, AAV-5, AAV-6, AAV-7, AAV-8, AAV-9, AAV-10 AAV-11, AAV-12, AAV-13, AAV-14, AAV-15 and AAV-16). In additional embodiments, rAAV particles comprise a pseudotyped rAAV particle comprised of a capsid protein of an AAV serotype selected from AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In additional embodiments, rAAV particles comprise a pseudotyped rAAV particle containing AAV-8 capsid protein. In additional embodiments, rAAV particles comprise a pseudotyped rAAV particle is comprised of AAV-9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In further embodiments, the capsid protein is a chimeric of 2 or more AAV capsid proteins from AAV serotypes selected from AAV1, AAV2, rAAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV-8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV-8 capsid protein and one or more AAV capsid proteins from an AAV serotype selected from AAV1, AAV2, AAV5, AAV6, AAV7, AAV9, AAV10, AAVrh.8, and AAVrh.10.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AA6, AAV7, AAV8, AAV9, AAVrh.8, and AAVrh.10.

Methods of Making rAAV Vectors

Another aspect of the present invention involves making molecules disclosed herein. In some embodiments, a molecule according to the invention is made by providing a nucleotide comprising the nucleic acid sequence encoding an AAV capsid protein; and using a packaging cell system to prepare corresponding rAAV particles with capsid coats made up of the capsid protein. In some embodiments, the nucleic acid sequence encodes a sequence having at least 60%, 70%, 80%, 85%, 90%, or 95%, preferably 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, or 95%, preferably 96%, 97%, 98%, 99% or 99.9%, identity to a particular sequence of the AAV capsid protein, while retaining (or substantially retaining) biological function of the AAV capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In certain embodiments, the cap and rep genes are provided by a packaging cell and not present in the viral genome.

In some embodiments, the nucleic acid encoding the capsid protein is cloned into an AAV Rep-Cap helper plasmid in place of the existing capsid gene. When introduced together into host cells, this plasmid helps package an rAAV genome into the capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In preferred embodiments, the rAAVs provide transgene delivery vectors that can be used in therapeutic and prophylactic applications, as discussed in more detail below. The rAAV vector also includes the regulatory control elements discussed supra to influence the expression of the RNA and/or protein products encoded by nucleic acids (transgenes) within target cells of the subject.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV capsid protein.

The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

The rAAV vector for delivering the transgene to target tissues, cells, or organs, may also have a tropism for that particular target tissue, cell, or organ, e.g. liver and/or muscle, in conjunction with the use of tissue-specific promoters as described herein. The construct can further include additional expression control elements such as introns that enhance expression of the transgene (e.g., introns such as the chicken β-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a certain embodiment, the constructs described herein comprise the following components (LSPX1): (1) AAV inverted terminal repeats (ITRs) that flank the expression cassette; (2) control elements, which include a) two tandem Mic/Bik enhancers, b) ApoE enhancer, c) human AAT promoter, d) a poly A signal, and e) optionally an intron; (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) two tandem Mic/Bik enhancers, b) ApoE enhancer, c) human AAT promoter, d) a rabbit β-globin poly A signal and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LSPX2): (1) AAV inverted terminal repeats (ITRs) that flank the expression cassette; (2) control elements, which include a) two tandem ApoE enhancers, b) human AAT promoter, c) a poly A signal; and d) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) two tandem ApoE enhancers, b) human AAT promoter, c) a poly A signal; and d) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LTP1): (1) AAV inverted terminal repeats (ITRs) that flank the expression cassette; (2) control elements, which include a) two tandem Mic/Bik enhancers, b) TBG promoter, c) human AAT (ΔATG) promoter, d) a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) two tandem Mic/Bik enhancers, b) TBG promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LTP2): (1) AAV inverted terminal repeats (ITRs) that flank the expression cassette; (2) control elements, which include a) ApoE enhancer, b) two tandem Mic/Bik enhancers, c) TBG promoter, d) human AAT (ΔATG) promoter, e) a poly A signal; and f) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) two tandem MckE enhancers, c) TBG promoter, d) human AAT (ΔATG) promoter, e) a poly A signal; and f) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LTP3): (1) AAV inverted terminal repeats (ITRs) that flank the expression cassette; (2) control elements, which include a) two tandem Mic/Bik enhancers, b) TBG promoter, c) human AAT (ΔATG) promoter, d) ApoE enhancer, e) a poly A signal; and f) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) two tandem MckE enhancers, b) TBG promoter, c) human AAT (ΔATG) promoter, d) ApoE enhancer, e) a poly A signal; and f) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP6): (1) AAV inverted terminal repeats (ITRs) that flank the expression cassette; (2) control elements, which include a) ApoE enhancer, b) three tandem MckE enhancers, c) CK8 promoter, d) human AAT (ΔATG) promoter, e) a poly A signal; and f) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) three tandem MckE enhancers, c) CK8 promoter, d) human AAT (ΔATG) promoter, e) a poly A signal; and f) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP13): (1) AAV inverted terminal repeats (ITRs) that flank the expression cassette; (2) control elements, which include a) ApoE enhancer, b) Spc5.12 promoter c) human AAT (ΔATG) promoter, d a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) Spc5.12 promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP14): (1) AAV inverted terminal repeats (ITRs) that flank the expression cassette; (2) control elements, which include a) minimal Spc5.12 promoter, b) human AAT (ΔATG) promoter, c) a poly A signal; and d) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) minimal Spc5.12 promoter, b) human AAT (ΔATG) promoter, c) a poly A signal; and d) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP15): (1) AAV inverted terminal repeats (ITRs) that flank the expression cassette; (2) control elements, which include a) ApoE enhancer, b) minimal Spc5.12 promoter c) human AAT (ΔATG) promoter, d a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) minimal Spc5.12 promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP18): (1) AAV inverted terminal repeats (ITRs) that flank the expression cassette; (2) control elements, which include a) ApoE enhancer, b) MckE enhancer, c) CK8 promoter, d) human AAT (ΔATG) promoter, e) a poly A signal; and f) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) MckE enhancer, c) CK8 promoter, d) human AAT (ΔATG) promoter, e) a poly A signal; and f) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP19): (1) AAV inverted terminal repeats (ITRs) that flank the expression cassette; (2) control elements, which include a) ApoE enhancer, b) CK8 promoter, c) human AAT (ΔATG) promoter, d) a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) CK8 promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LMTP20): (1) AAV inverted terminal repeats (ITRs) that flank the expression cassette; (2) control elements, which include a) ApoE enhancer, b) MhcE enhancer, c) MckE enhancer, d) CK8 promoter, e) human AAT (ΔATG) promoter, f) a poly A signal; and g) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) MhcE enhancer, c) MckE enhancer, d) CK8 promoter, e) human AAT (ΔATG) promoter, f) a poly A signal; and g) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LBTP1): (1) AAV inverted terminal repeats (ITRs) that flank the expression cassette; (2) control elements, which include a) ApoE enhancer, b) minimal SP7/Osx promoter c) human AAT (ΔATG) promoter, d) a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) minimal SP7/Osx promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

In a certain embodiment, the constructs described herein comprise the following components (LBTP2): (1) AAV inverted terminal repeats (ITRs) that flank the expression cassette; (2) control elements, which include a) ApoE enhancer, b) SP7/Osx promoter c) human AAT (ΔATG) promoter, d) a poly A signal; and e) optionally an intron; and (3) transgene providing (e.g., coding for) one or more RNA or protein products of interest. In another embodiment, the constructs described herein comprise the following components: (1) AAV2 inverted terminal repeats that flank the expression cassette; (2) control elements, which include a) ApoE enhancer, b) SP7/Osx promoter, c) human AAT (ΔATG) promoter, d) a poly A signal; and e) optionally a chimeric intron derived from human β-globin and Ig heavy chain, or other intron; and (3) transgene providing (e.g., coding for) one or more RNA or protein products of interest, such as those in Tables 4A-4D.

The tandem and composite promoters described herein result in preferred transcription start sites within the promoter region. See for example, the results of Example 10 and Table 14. Thus, in certain embodiments, the constructs described herein have a tandem or composite nucleic acid regulatory sequence that comprises an hAAT promoter (particularly a modified start codon hAAT promoter) and has a transcription start site of TCTCC (SEQ ID NO: 43) (corresponding to nucleotides 1541-1545 of LMTP6 SEQ ID NO: 6), or the active transcription site found in hAAT (corresponding to 355-359 of SEQ ID NO: 11 or SEQ ID NO: 12) or GGTACAATGACTCCTTTCG (SEQ ID NO: 41), which corresponds to nucleotides 139-157 of SEQ ID NO: 11, or GGTACAGTGACTCCTTTCG (SEQ ID NO: 42), which corresponds to nucleotides 139-157 of SEQ ID NO: 12. In other embodiments, the constructs described herein have a tandem or composite regulatory sequence that comprises a CK8 promoter and has a transcription start site at TCATTCTACC (SEQ ID NO: 46), which corresponds to nucleotides 377-386 of SEQ ID NO: 16, particularly starting at the nucleotide corresponding to nucleotide 377 of SEQ ID NO: 16 or corresponding to nucleotide 1133 of SEQ ID NO: 6.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or muscle or other cell types may be used, for example, but not limited, to HuH-7, HEK293, fibrosarcoma HT-1080, HKB-11, C2C12 myoblasts, and CAP cells. Once expressed, characteristics of the expressed product (transgene product) can also be determined, including serum half-life, functional activity of the protein (e.g. enzymatic activity or binding to a target), determination of the glycosylation and tyrosine sulfation patterns, and other assays known in the art for determining protein characteristics.

Provided are methods of manufacturing a recombinant AAV comprising culturing a host cell capable of producing a recombinant AAV described herein under conditions appropriate for production of the recombinant AAV comprising an artificial genome with an expression cassette comprising a synthetic promoter operably linked to a transgene. In particular, the method provides (1) culturing a host cell containing (i) an artificial genome comprising AAV ITRs flanking a recombinant cis expression cassette which comprises a nucleic acid regulatory element comprising a composite nucleic acid regulatory element as disclosed herein operably linked to a transgene; (ii) a trans expression cassette lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and (iii) sufficient adenovirus helper functions to permit replication and packaging of the artificial genome by the AAV capsid protein; and (2) recovering recombinant AAV encapsidating the artificial genome from the cell culture. Also provided are host cells containing (i) an artificial genome comprising AAV ITRs flanking a recombinant cis expression cassette which comprises a composite nucleic acid regulatory element disclosed herein operably linked to a transgene; (ii) a trans expression cassetted lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and, optionally, (iii) sufficient adenovirus helper functions to permit replication and packaging of the artificial genome by the AAV capsid protein In particular embodiments, the composite nucleic acid regulatory element is LSPX1, LSPX2, LTP1, LTP2, LTP3, LMTP6, LMTP13, LMTP14, LMTP15, LMTP18, LMTP19, LMTP20, LBTP1, or LBTP2 of Table 1. In particular embodiments, the composite nucleic acid regulatory element comprises or consists of SEQ ID NO: 1, SEQ ID NO: 2, SEQ ID NO: 3, SEQ ID NO: 4, SEQ ID NO: 5, SEQ ID NO: 6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31. In certain embodiments, the artificial genome comprises a transgene encoding one of the therapeutics listed in Tables 4A-4D.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 4A-4D below provides a list of transgenes that may be used in any of the rAAV vectors described herein, in particular, in the novel insertion sites described herein, preferably to treat or prevent the disease with which it is associated, also listed in Tables 4A-4D. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 4A

| | | Possible |
|---|---|---|
| | | AAV |
| | | serotype for |
| | | delivery of |
| Disease | Transgene | transgene |
| MPS I | alpha-L-iduronidase (IDUA) | AAV9 |
| MPS II (Hunter | iduronate-2-sulfatase (IDS) | AAV9 |
| Syndrome) | | |
| ceroid lipofuscinosis | (CLN1, CLN2, CLN10, CLN13), a soluble | AAV9 |
| (Batten disease) | lysosomal protein (CLN5), a protein in the | |
| | secretory pathway (CLN11), two cytoplasmic | |
| | proteins that also peripherally associate with | |
| | membranes (CLN4, CLN14), and many | |
| | transmembrane proteins with different subcellular | |
| | locations (CLN3, CLN6, CLN7, CLN8, CLN12) | |
| MPS IIIa (Sanfilippo | heparan sulfate sulfatase (also called N- | AAV9, Rh10 |
| type A Syndrome) | sulfoglucosamine sulfohydrolase (SGSH)) | |
| MPS IIIB (Sanfilippo | N-acetyl-alpha-D-glucosaminidase (NAGLU) | AAV9 |
| type B Syndrome) | | |
| MPS VI (Maroteaux- | arylsulfatase B | AAV8 |
| Lamy Syndrome) | | |
| MPS IVA (Morquio | GALNS | AAV8 |
| syndrome type A) | | |
| MPS IVA (Morquio | GLB1 | AAV8 |
| syndrome type B) | | |
| Osteogenesis | COL1A1 and/or COL1A2 | AAV8 |
| Imperfecta Type I, II, | | |
| III, or IV | | |
| Osteogenesis | IFITM5 | AAV8 |
| Imperfecta Type V | | |
| Osteogenesis | SERPINF1 | AAV8 |
| Imperfecta Type VI | | |
| Osteogenesis | CRTAP | AAV8 |
| Imperfecta Type VII | | |
| Osteogenesis | LEPRE1 and/or P3H1 | AAV8 |
| Imperfecta Type VIII | | |
| Osteogenesis | PPIB | AAV8 |
| Imperfecta Type IX | | |
| Gaucher disease (type | Glucocerebrosidase, GBA1 | AAV9 |
| 1, II and III) | | |
| Parkinson's Disease | Glucocerebrosidase; GBA1 | AAV9 |
| Parkinson's Disease | dopamine decarboxylase | AAV2 |
| Pompe | acid maltase; GAA | AAV9 |
| Metachromatic | Aryl sulfatase A | Rh10 |
| leukodystrophy | | |
| MPS VII (Sly | beta-glucuronidase | |
| syndrome) | | |
| MPS VIII | glucosamine-6-sulfate sulfatase | |
| MPS IX | Hyaluronidase | |
| Niemann-Pick disease | Sphingomyelinase | |
| Niemann-Pick disease | a npc1 gene encoding a | |
| without | cholesterol metabolizing enzyme | |
| sphingomyelinase | | |
| deficiency | | |
| Tay-Sachs disease | Alpha subunit of beta-hexosaminidase | |
| Sandhoff disease | both alpha and beta subunit of beta- | |
| | hexosaminidase | |
| Fabry Disease | alpha-galactosidase | |
| Fucosidosis | Fucosidase (FUCA1 gene) | |
| Alpha-mannosidosis | alpha-mannosidase | |
| Beta-mannosidosis | Beta-mannosidase | |
| Wolman disease | cholesterol ester hydrolase | |
| Parkinson's disease | Neurturin | |
| Parkinson's disease | glial derived growth factor (GDGF) | |
| Parkinson's disease | tyrosine hydroxylase | |
| Parkinson's disease | glutamic acid decarboxylase. | |
| | fibroblast growth factor-2 (FGF-2) | |
| | brain derived growth factor (BDGF) | |
| No disease listed | neuraminidase deficiency with betagalactosidase | |
| (Galactosialidosis | deficiency | |
| (Goldberg syndrome)) | | |
| Spinal Muscular | SMN | AAV9 |
| Atrophy (SMA) | | |
| Friedreich's ataxia | Frataxin | AAV9 |
| | | PHP.B |
| Amyotrophic lateral | SOD1 | Rh10 |
| sclerosis (ALS) | | |
| Glycogen Storage | Glucose-6-phosphatase | AAV8 |
| Disease 1a | | |
| XLMTM | MTM1 | AAV8 or |
| | | AAV9 |
| Crigler Najjar | UGT1A1 | AAV8 |
| CPVT | CASQ2 | AAV9 |
| Rett syndrome | MECP2 | AAV9 |
| Achromatopsia | CNGB3, CNGA3, GNAT2, PDE6C | AAV8 |
| Choroidermia | CDM | AAV8 |
| Danon Disease | LAMP2 | AAV9 |

TABLE 4B

| | | Possible AAV |
|---|---|---|
| | | serotype for |
| | | delivery of |
| Disease | Transgene | transgene |
| Cystic Fibrosis | CFTR | AAV2 |
| Duchenne Muscular Dystrophy | Mini-/Micro-Dystrophin Gene | AAV2, AAV8, |
| | | AAV9 |
| Limb Girdle Muscular Dystrophy Type | human-alpha-sarcoglycan | AAV1 |
| 2C\|Gamma-sarcoglycanopathy | | |
| Advanced Heart Failure | SERCA2a | AAV6 |
| Rheumatoid Arthritis | TNFR:Fc Fusion Gene | AAV2 |
| Leber Congenital Amaurosis | GAA | AAV1 |
| Limb Girdle Muscular Dystrophy Type | gamma-sarcoglycan | AAV1 |
| 2C\|Gamma-sarcoglycanopathy | | |
| Retinitis Pigmentosa | hMERTK | AAV2 |
| Age-Related Macular Degeneration | sFLT01 | AAV2 |
| Becker Muscular Dystrophy and | huFollistatin344 | AAV1 |
| Sporadic Inclusion Body Myositis | | |
| Parkinson's Disease | GDNF | AAV2 |
| Metachromatic Leukodystrophy (MLD) | cuARSA | AAVrh.10 |
| Hepatitis C | anti-HCV shRNA | AAV8 |
| Limb Girdle Muscular Dystrophy Type | hSGCA | AAVrh74 |
| 2D | | |
| Human Immunodeficiency Virus | PG9DP | AAVI |
| Infections; HIV Infections (HIV-1) | | |
| Acute Intermittant Porphyria | PBGD | AAV5 |
| Leber's Hereditary Optical Neuropathy | P1ND4v2 | AAV2 |
| Alpha-1 Antitrypsin Deficiency | alpha1AT | AAVrh10 |
| Pompe Disease | hGAA | AAV9 |
| X-linked Retinoschisis | RS1 | AAV8 |
| Choroideremia | hCHM | AAV2 |
| Giant Axonal Neuropathy | JeT-GAN | AAV9 |
| Duchenne Muscular Dystrophy | micro-Dystrophin | AAVrh74 |
| X-linked Retinoschisis | hRS1 | AAV2 |
| Squamous Cell Head and Neck Cancer; | hAQP1 | AAV2 |
| Radiation Induced Xerostomia | | |
| Hemophilia B | Factor IX | AAVrh10/ |
| | | Rh74 |
| Homozygous FH | hLDLR | AAV8 |
| Dysferlinopathies | rAAVrh74.MHCK7.DYSF.DV | AAVrh74 |
| Hemophilia B | AAV6 ZFP nuclease | AAV6 |
| MPS I | AAV6 ZFP nuclease | AAV6 |
| Rheumatoid Arthritis | NF-kB.IFN-β | AAV5 |
| Batten/CLN6 | CLN6 | AAV9 |
| Sanfilippo Disease Type A | hSGSH | AAV9 |
| Osteoarthritis | 5IL-1Ra | AAV2.5 |
| Achromatopsia | CNGA3 | AAV2tYF |
| Achromatopsia | CNGB3 | AAV8 |
| Ornithine Transcarbamylase (OTC) | OTC | scAAV8 |
| Deficiency | | |
| Hemophilia A | Factor VIII | LK03/AAV3B |
| Mucopolysaccharidosis II | ZFP nuclease | AAV6 |
| Hemophilia A | ZFP nuclease | AAV6 |
| Wet AMD | anti-VEGF | AAV8 |
| X-Linked Retinitis Pigmentosa | RPGR | AAV2 |
| Mucopolysaccharidosis Type VI | hARSB | AAV8 |
| Leber Hereditary Optic Neuropathy | ND4 | AAV2 |
| X-Linked Myotubular Myopathy | MTM1 | AAV8 |
| Crigler-Najjar Syndrome | UGT1A1 | AAV8 |
| Achromatopsia | CNGB3 | AAV8 |
| Retinitis Pigmentosa | hPDE6B | AAV5 |
| X-Linked Retinitis Pigmentosa | RPGR | AAV2tYF |
| Mucopolysaccharidosis Type 3 B | hNAGLU | AAV9 |
| Duchenne Muscular Dystrophy | GALGT2 | AAVrh74 |
| Arthritis, Rheumatoid; Arthritis, | TNFR:Fc Fusion Gene | AAV2 |
| Psoriatic; Ankylosing Spondylitis | | |
| Idiopathic Parkinson's Disease | Neurturin | AAV2 |
| Alzheimer's Disease | NGF | AAV2 |
| Human Immunodeficiency Virus | tgAAC09 | AAV2 |
| Infections; HIV Infections (HIV-1) | | |
| Familial Lipoprotein Lipase Deficiency | LPL | AAV1 |
| Idiopathic Parkinson's Disease | Neurturin | AAV2 |
| Alpha-1 Antitrypsin Deficiency | hAAT | AAV1 |
| Leber Congenital Amaurosis (LCA) 2 | hRPE65v2 | AAV2 |
| Batten Disease; Late Infantile | CLN2 | AAVrh.10 |
| Neuronal Lipofuscinosis | | |
| Parkinson's Disease | GAD | AAV2 |
| Sanfilippo Disease Type A/ | N-sulfoglucosamine | AAVrh.10 |
| Mucopolysaccharidosis Type IIIA | sulfohydrolase (SGSH) gene | |
| Congestive Heart Failure | SERC2a | AAV1 |
| Becker Muscular Dystrophy and | rAAV1.CMV.huFollistatin344 | AAV1 |
| Sporadic Inclusion Body Myositis | | |
| Parkinson's Disease | hAADC-2 | AAV2 |
| Choroideremia | REP1 | AAV2 |
| CEA Specific AAV-DC-CTL | CEA | AAV2 |
| Treatment in Stage IV Gastric Cancer | | |
| Gastric Cancer | MUC1-peptide-DC-CTL | |
| Leber's Hereditary Optical Neuropathy | scAAV2-P1ND4v2 | scAAV2 |
| Aromatic Amino Acid Decarboxylase | hAADC | AAV2 |
| Deficiency | | |
| Hemophilia B | Factor IX | AAVrh10 |
| Parkinson's Disease | AADC | AAV2 |
| Leber Hereditary Optic Neuropathy | Genetic: GS010\|Drug: Placebo | AAV2 |
| SMA—Spinal Muscular Atrophy\|Gene | SMN | AAV9 |
| Therapy | | |
| Hemophilia A | B-Domain Deleted Factor VIII | AAV8 |
| MPS I | IDUA | AAV9 |
| MPS II | IDS | AAV9 |
| CLN3-Related Neuronal Ceroid- | CLN3 | AAV9 |
| Lipofuscinosis (Batten) | | |
| Limb-Girdle Muscular Dystrophy, Type | hSGCB | rh74 |
| 2E | | |
| Alzheimer Disease | APOE2 | rh10 |
| Retinitis Pigmentosa | hMERKTK | AAV2 |
| Retinitis Pigmentosa | RLBP1 | AAV8 |
| Wet AMD | Anti-VEGF antibody | AAV2.7m8 |

TABLE 4C

| Antigens | | Antibodies | INDICATIONS |
|---|---|---|---|
| Nervous System | Amyloid beta | solanezumab | Alzheimer's Disease |
| Targets | (Aβ or Abeta) | GSK933776 | |
| | peptides | lecanemab | |
| | derived from | | |
| | APP | | |
| | Sortilin | AL-001 | Frontotemporal dementia |
| | | | (FTD) |
| | Tau protein | ABBV-8E12 | Alzheimer's, Progressive |
| | | UCB-0107 | supranuclear palsy, |
| | | NI-105 (BIIB076) | frontotemporal |
| | | | demential, chronic |
| | | | traumatic |
| | | | encephalopathy, Pick's |
| | | | complex, primary age- |
| | | | related taupathy |
| | Semaphorin- | VX15/2503 | Huntington's disease, |
| | 4D | | juvenile Huntington's |
| | (SEMA4D) | | disease |
| | alpha- | prasinezumab | Parkinson's disease, |
| | synuclein | NI-202 (BIIB054) | synucleinopathies |
| | | MED-1341 | |
| | superoxide | NI-204 | ALS, Alzheimer's |
| | dismutase-1 | | Disease |
| | (SOD-1) | | |
| | CGRP | eptinezumab, | Migraines, Cluster |
| | Receptor | fremanezumab | headaches |
| | | galcanezumab | |
| Ocular Anti- | VEGF | sevacizumab | diabetic retinopathy |
| Angiogenic | | | (DR), myopic choroidal |
| Targets | | | neovascularization |
| | | | (mCNV), age-related |
| | | | macular degeneration |
| | | | (AMD), macular edema |
| | erythropoietin | LKA-651 | retinal vein occlusion |
| | receptor | | (RVO), wet AMD, |
| | | | macular edema |
| | Amyloid beta | solanezumab | Dry AMD |
| | (Aβ or Abeta) | GSK933776 | |
| | peptides | lecanemab | |
| | derived from | | |
| | APP | | |
| | activin | ascrinvacumab | neovascular age-related |
| | receptor like | | macular degeneration |
| | kinase 1 | | |
| | (ALK1) | | |
| | complement | tesidolumab | dry AMD, uveitis |
| | component 5 | ravulizumab | |
| | (C5) | | |
| | endoglin | carotuximab | wet AMD and other |
| | (END or | | retinal disorders caused |
| | CD105) | | by increased |
| | | | vascularization |
| | complement | ANX-007 | glaucoma |
| | component | | |
| | 1Q (C1Q) | | |
| TNF-alpha | | adalimumab (HUMIRA ®) | uveitis |
| | | infliximab | |
| | | (REMICADE ®) | |
| | | golimumab | |
| Repulsive guidance molecule-A | | elezanumab | multiple sclerosis |
| Transthyretin (TTR) | | NI-301 | amyloidosis |
| | | PRX-004 | |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Neuromyelitis | interleukin | Satralizumab | NMO, DR, DME, uveitis |
| optica | receptor 6 | Sarilumab | |
| (NMO)/Uveitis | (IL6R) | Tocilizumab | |
| targets | Interleukin 6 | siltuximab, | NMO, DR, DME, uveitis |
| | | clazakizumab | |
| | | sirukumab | |
| | | olokizumab | |
| | | gerilimzumab | |
| | CD19 | inebilizumab | NMO |

| | | |
|---|---|---|
| Integrin beta 7 | etrolizumab | ulcerative colitis, Crohn's |
| | | disease |
| Sclerostin | romosozumab | Osteoporosis, abnormal |
| | (EVENITY ®) | bone loss or weakness |
| Complement Component 5 | ravulizumab | Myasthenia Gravis |
| Interleukin receptor 6 (IL6R) | Satralizumab | Adverse immune |
| | Sarilumab | responses (e.g. cytokine |
| | Tocilizumab | storm, CAR-T therapy) |
| Interleukin 6 (IL6) | siltuximab, | |
| | clazakizumab | |
| | sirukumab | |
| | olokizumab | |
| | gerilimzumab | |
| Immuno globin E (IgE) | omolizumab | Asthma, COPD, |
| | | eosinophilic asthma, |
| | | chronic idiopathic |
| | | urticaria |
| Thymic stromal lymphopoietin | tezelipumab | Asthma, COPD |
| (TSLP) | | |
| Interleukin 5 (IL5) | benralizumab | Asthma, COPD |
| Interleukin 5 receptor (IL5R) | reslizumab | Asthma, COPD, |
| | | eosinophilic asthma |
| Interleukin 13 (IL13) | tralokinumab | Atopic dermatitis |
| Interleukin 31 recptor alpha | nemolizumab | Atopic dermatitis |
| (IL31RA) | | |

TABLE 4D

| Antigens | | Antibodies | INDICATIONS |
|---|---|---|---|
| Nervous System | Amyloid beta | Aducanumab | Alzheimer's Disease |
| Targets | (Aβ or Abeta) | crenezumab | |
| | peptides | gantenerumab | |
| | Tau protein | anti-TAU | Alzheimer's, Progressive |
| | | | supranuclear palsy, |
| | | | frontotemporal demential, |
| | | | chronic traumatic |
| | | | encephalopathy, Pick's |
| | | | complex, primary age- |
| | | | related taupathy |
| | CGRP Receptor | erenumab | Migraine |
| | | (AIMOVIG ™) | |
| Interleukins or | IL-17A | ixekizumab (TALTZ ®) | Plaque psoriasis, psoriatic |
| interleukin | | secukinumab | arthritis, ankylosing |
| receptors | | (COSENTYX ®) | sponylitis |
| | IL-5 | mepolizumab | Asthma |
| | | (NUCALA ®) | |
| | IL-12/IL-23 | ustekinumab | Psoriasis & Crohn's |
| | | (STELARA ®) | disease |
| | IL-4R | dupilumab | Atopic dermatitis |
| Integrin | | vedolizumab | Ulcerative colitis & |
| | | (ENTYVIO ®) | Crohn's disease |
| | | Natalizumab (anti- | Multiple sclerosis & |
| | | integrin alpha 4) | Crohn's disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab (XGEVA ® | Osteoporosis, increasing |
| | | and | bone mass in breast and |
| | | PROLIA ®) | prostate cancer patients, & |
| | | | preventing skeletal-related |
| | | | events due to bone |
| | | | metastasis |
| PD-1, or PD-L1 or PD-L2 | | nivolumab (OPDIVO ®) | Metastatic melanoma, |
| | | pembrolizumab | lymphoma, non-small cell |
| | | (KEYTRUDA ®) | lung carcinoma |
| BLyS (B-lymphocyte stimulator, | | belimumab | Systemic lupus |
| also known as B-cell activating | | (BENLYSTA ®) | erythromatosis |
| factor (BAFF)) | | | |
| Ocular Targets | VEGF | ranibizumab | Wet AMD |
| | | (LUCENTIS ®) | |
| | | bevacizumab | |
| | | (AVASTIN ®) | |
| | | brolucizumab | |
| | Factor D | lampalizumab | Dry AMD |
| | MMP9 | andecaliximab | |
| TNF-alpha | | adalimumab | Rheumatoid arthritis, |
| | | (HUMIRA ®) and | psoriatic arthritis, |
| | | infliximab | askylosing spondylitis, |
| | | (REMICADE ®) | Crohn's disease, plaque |
| | | | psoriasis, ulcerative colitis |
| Plasma Protein | C5, C5a | eculizumab (SOLIRIS ®) | Paroxysmal nocturnal |
| targets | | | hemoglobinuria, atypical |
| | | | hemolytic uremic |
| | | | syndrome, complement- |
| | | | mediated thrombotic |
| | | | microangiopathy |
| | Plasma | lanadelumab | Hereditary angioedema |
| | kallikrein | | (HAE) |

In one example, a rAAV vector comprising a transgene encoding glial derived neurotrophic factor (GDNF) finds use in treating/preventing/managing Parkinson's disease. In another example, a rAAV comprising a transgene encoding an anti-kallikrein antibody, such as lanadelumab finds use in treating/preventing/managing hereditary angioedema (HAE). In still another example, a rAAV comprising a transgene encoding a lysosomal enzyme finds use in treating/preventing/managing mucopolysaccharidosis. Generally, the rAAV vector is administered systemically, and following transduction, the vector's production of the protein product is enhanced by an expression cassette employing engineered liver-specific and optionally muscle-specific or bone-specific nucleic acid regulatory elements. For example, the rAAV vector may be provided by intravenous, intramuscular, and/or intra-peritoneal administration.

With respect to the therapeutic antibodies in Tables 4C and 4D, the expression cassettes comprising the regulatory sequences operably linked to the transgene encoding the therapeutic antibody may be packaged in an rAAV for delivery that preferably has an AAV8 capsid, an AAV9 capsid or an AAVrh10 capsid for targeting or expression in liver and/or muscle cells.

In some aspects, the rAAVs of the present invention find use in delivery to target tissues associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject the an rAAV where the expression cassette comprises a nucleic acid regulatory element LSPX1, LSPX2, LTP1, LTP2, LTP3, LMTP6, LMTP13, LMTP14, LMTP15, LMTP18, LMTP19, LMTP20, LBTP1, or LBTP2 such as in Table 1 operably linked to a transgene.

Following transduction of target cells, the expression of the protein product is enhanced by employing such liver-specific expression cassettes. Such enhancement may be measured by the following non-limiting list of determinations such as 1) protein titer by assays known to the skilled person, not limited to sandwich ELISA, Western Blot, histological staining, and liquid chromatography tandem mass spectrometry (LC-MS/MS); 2) protein activity, by assays such as binding assays, functional assays, enzymatic assays and/or substrate detection assays; and/or 3) serum half-life or long-term expression. Enhancement of transgene expression may be determined as efficacious and suitable for human treatment (Hintze, J. P. et al, Biomarker Insights 2011:6 69-78). Assessment of the quantitative and functional properties of a transgene using such in vitro and in vivo cellular, blood and tissue studies have been shown to correlate to the efficacy of certain therapies (Hintze, J. P. et al, 2011, supra), and are utilized to evaluate response to gene therapy treatment of the transgene with the vectors described herein.

rAAV vectors of the invention also can facilitate delivery, in particular, targeted delivery, of transgenes operably linked to the chimeric regulatory sequences described herein, including but not limited to oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. In some embodiments, the rAAV molecule may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some preferred embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁹ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In certain embodiments, the transgene is administered intravenously even if intended to be expressed in the CNS, for example, by forming a depot in the liver where the transgene is expressed and secreted into the bloodstream.

In certain embodiments, the agents of the invention are administered intravenously or intramuscularly and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Intl. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e. g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, the rAAVs can be used for in vivo delivery of transgenes for scientific studies such as gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention comprising a transgene cassette wherein the transgene expression is driven by the chimeric regulatory elements described herein. In preferred embodiments, the pharmaceutical composition comprises rAAV combined with a pharmaceutically acceptable carrier for administration to a subject. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously or intramuscularly. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In preferred embodiments, the agent of the invention is substantially purified (i.e., substantially free from substances that limit its effect or produce undesired side-effects). In a specific embodiment, the host or subject is an animal, preferably a mammal such as non-primate (e.g., cows, pigs, horses, cats, dogs, rats etc.) and a primate (e.g., monkey such as, a cynomolgous monkey and a human). In a preferred embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

### 6.1. Example 1—Construction of Tandem Promoters

### 6.1.1. Liver-Specific Promoters (LSPX)

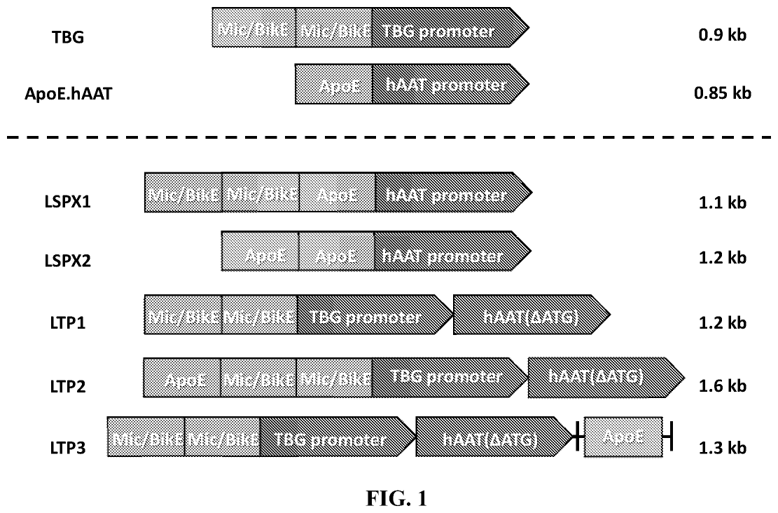

FIG. 1 depicts various arrangements of tandem promoters for use with any transgene. The promoter sequences were rationally designed promoters to confer expression of gene therapy vectors specifically within hepatocytes. The sequences of these elements were derived, in part, from canonical promoters such as TBG and hAAT promoters, and cassettes were cloned into AAV (cis) plasmids using standard molecular biology techniques. For example, the ApoE.hAAT promoter used in the Examples herein was constructed as in FIG. 1, where one hepatic control region which contains an ApoE enhancer (SEQ ID NO: 9) upstream of the hAAT promoter sequence (SEQ ID NO: 11). The following is a description of each liver-specific promoter:

LSPX1: This sequence contains two copies of the alpha-Mic/Bik enhancer (derived from the TBG promoter), the ApoE enhancer, and the hAAT promoter. A chimeric β-globin/Ig intron was placed downstream (3′) of the promoter sequence.

LSPX2: this promoter contains two copies of the ApoE enhancer and hAAT promoter. The canonical hAAT promoter cassette only contains one copy of the ApoE enhancer. A chimeric β-globin/Ig intron was placed downstream (3′) of the promoter sequence. 6.1.2. Liver-Specific Tandem Promoters (LTP)

A novel approach to express a single transgene from two promoters (tandem systems) was employed by depleting the 3′ promoter of ‘ATG’ sequences (FIG. 1). The following promoter cassettes utilize this strategy to express transgenes specifically from the liver:

LTP1: This sequence contains two copies of the alpha-Mic/Bik enhancer followed by the TBG promoter. Downstream of the TBG promoter is the hAAT promoter sequence that has been depleted of ‘ATG’ sequences (i.e. hAAT-ΔATG). Not wishing to be bound by theory, the tandem promoters having ATG sites eliminated from the downstream promoter allows for expression of two mRNA transcripts—one driven by each of the promoters depending on host cell transcription machinery-however, protein translation initiation will occur at the single, intended start codon of the protein coding sequence in the cassette. This strategy should provide more efficient and robust expression compared to tandem promoters that contain superfluous ATG sites upstream of the protein initiation codon. A chimeric β-globin/Ig intron was placed downstream (3′) of the promoter sequence.

LTP2: This sequence is similar to LTP1 (contains a hAAT-ΔATG downstream of the TBG promoter) with the exception that it contains the ApoE enhancer further upstream of the alpha-Mic/Bik enhancers. A chimeric β-globin/Ig intron was placed downstream (3′) of the promoter sequence.

LTP3: This design is similar to LTP1 (contains a hAAT-ΔATG downstream of the TBG promoter). However, it contains a synthetic intron harboring the ApoE enhancer downstream of the other promoter elements, instead of the chimeric β-globin/Ig intron.

### 6.1.3. Liver/Muscle Dual-Specific Tandem Promoters (LMTP)

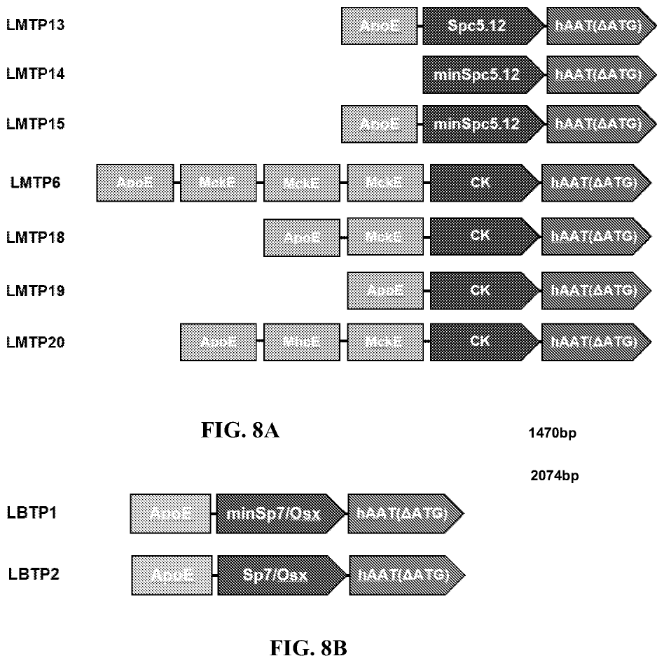

An approach to express a single transgene from two promoters (tandem systems) was employed by depleting the 3′ promoter of ‘ATG’ sequences (FIGS. 5 and 8A). The following promoter cassettes utilize this strategy to achieve dual transgene expression in the liver and muscle.

LMTP6: This is a tandem promoter cassette that demonstrates expression within both liver and muscle cells. It contains the ApoE enhancer followed by the complete CK8 promoter cassette (which contains three copies of the Mck Enhancer (MckE) upstream of a CK8 promoter sequence). Downstream of the CK8 promoter is the hAAT promoter depleted of ATG sites. This cassette theoretically allows expression of two transcripts. The CK8 promoter will express a transcript within muscle cell types, while the hAAT-ΔATG produces a transcript within hepatocytes. Once again, the first ‘ATG’ initiation codon encountered in both transcripts occurs at the intended site of translation.

LMTP13: This tandem promoter cassette was engineered to express transgene within both liver and muscle cells. It contains the ApoE enhancer followed by the complete Spc5.12 promoter cassette. Downstream of the Spc5.12 promoter is the hAAT promoter depleted of ATG sites. Similar to the examples above, all of cassettes LMTP13, LMTP14, LMTP15, LMTP18, LMTP19, and LMTP20 theoretically allow expression of two transcripts, one specific for muscle and one for liver cell expression. A chimeric β-globin/Ig intron was placed downstream (3′) of the promoter sequence.

LMTP14: This sequence contains a minimal Spc5.12 promoter upstream of a hAAT promoter depleted of ATG sites. A VH4 intron was placed downstream (3′) of the promoter sequence.

LMTP15: This sequence contains the ApoE enhancer followed by the minimal Spc5.12 promoter. A hAAT-ΔATG is placed downstream of the minimal Spc5.12 promoter. A VH4 intron was placed downstream (3′) of the promoter sequence.

LMTP18: This tandem promoter sequence was constructed to contain, from 5′ to 3′, an ApoE enhancer upstream of one copy of an Mck Enhancer (MckE), CK8 promoter, and finally the hAAT-ΔATG promoter. A chimeric β-globin/Ig intron was placed downstream (3′) of the promoter sequence.

LMTP19: This tandem promoter sequence was constructed to contain, from 5′ to 3′, an ApoE enhancer upstream of a CK8 promoter (devoid of Mck Enhancer elements), followed by the hAAT-ΔATG promoter.

LMTP20: This tandem promoter sequence was constructed to contain, from 5′ to 3′, an ApoE enhancer upstream of a two copies of an Mck Enhancer (MckE), CK8 promoter, then followed by the hAAT-ΔATG promoter.

AAV proviral (cis) plasmids containing these sequence elements were also packaged into infectious vector particles and purified as products for gene therapy.

### 6.1.4. Liver/Bone Dual-Specific Tandem Promoters (LBTP)

Bone/liver dual-specific tandem promoters were designed and recombinantly engineered into Cis plasmids. See FIG. 8B.

LBTP1: A minimal Sp7/Osx promoter fragment driving osteoblast-specific expression was determined to be a transcriptionally active fragment of the Sp7/Osx promoter (Lu, X., et al. JBC 281, 6297-6306, Jan. 12, 2006, herein incorporated by reference in its entirety). The LBTP1 sequence contains one copy of the minimal Sp7 promoter fragment (SEQ ID NO: 30) flanked 5′ by a liver-specific ApoE enhancer/hepatic control region, and 3′ by a hAAT promoter depleted of ATG trinucleotides (hAAT-ΔATG) to drive hepatocyte-specific expression, as illustrated in FIG. 1. A chimeric β-globin/Ig intron was placed downstream (3′) of the promoter sequence, i.e. downstream of the hAATΔATG.

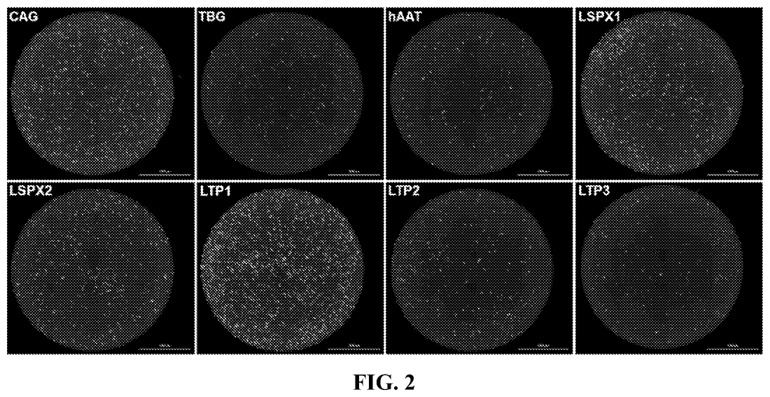

LBTP2: A full-length Sp7/Osx promoter (Lu, X., et al. JBC 281, 6297-6306, Jan. 12, 2006, herein incorporated by reference in its entirety) (SEQ ID NO: 31) was flanked 5′ by a liver-specific ApoE enhancer/hepatic control region, and 3′ by a hAAT promoter depleted of ATG sites (hAAT-ΔATG) to drive hepatocyte-specific expression, as illustrated in FIG. 2. Not wishing to be bound by theory, this design may allow for expression of two mRNA transcripts (one driven by each of the bone promoter and the liver promoter), however protein translation initiation will only occur at the single, intended start codon of the protein coding sequence. A chimeric β-globin/Ig intron was placed downstream (3′) of the promoter sequence, i.e. downstream of the hAAT-ΔATG.

Cis plasmids comprising the LBTP1 and LBTP2 promoter were constructed to express a lysosomal enzyme (transgene) and the entire cassette was flanked by AAV ITRs. LBTP1 or LBTP2 promoter are expected to confer expression of the gene therapy vector specifically in osteoblasts and hepatocytes. AAV proviral (cis) plasmids containing these sequence elements can be packaged into infectious vector particles and purified as products for gene therapy.

### 6.2. Example 2—GFP Expression Driven by Liver-Specific Promoters

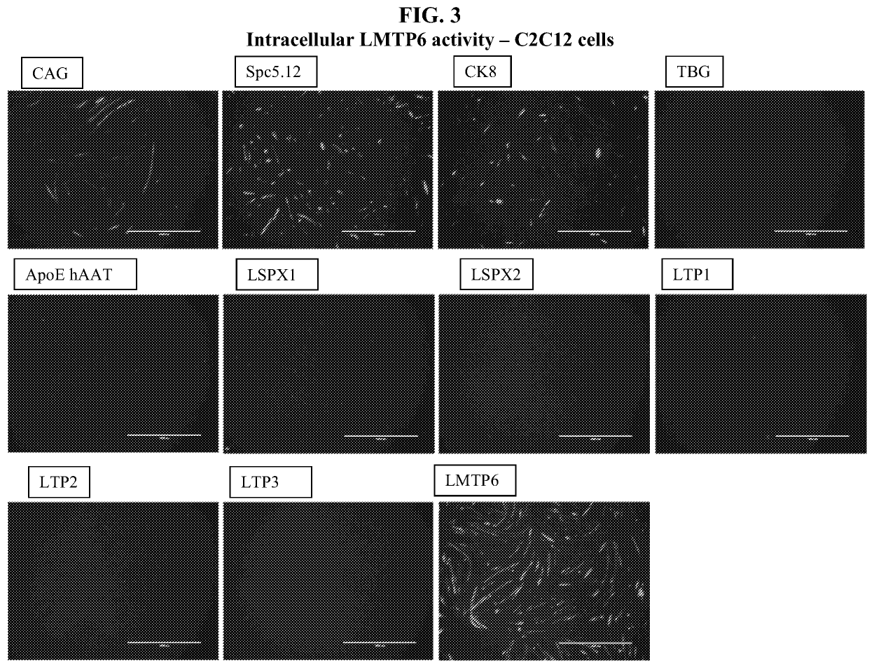

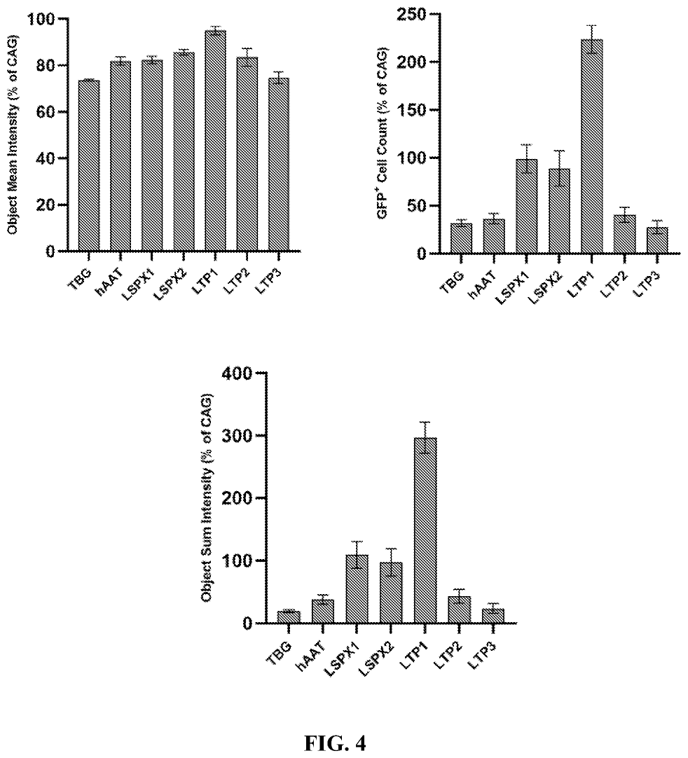

Eight GFP-expressing constructs (AAV cis plasmids) as depicted in FIG. 1, including a construct utilizing the universal CAG promoter (SEQ ID NO: 17) operably linked to GFP, were transfected into HuH7 and C2C12 cells. Briefly, Huh-7 cells and C2C12 cells were maintained at 37° C. in a 5% CO2 incubator in Dulbecco's modified Eagle's medium (Corning, Corning, N.Y., USA) supplemented with 10% fetal bovine serum and penicillin-streptomycin (100 U/ml). Transient transfection was respectively performed with 1 μg of each vector plasmid expressing GFP using Lipofectamine 3000 (Invitrogen, Carlsbad, Calif., USA) in a 6 well plate. After 48 hr transfection, cell pellet and medium were collected and stored at −20° C. until further processing. Each construct, except LTP3, additionally contains a chimeric intron derived from human β-globin and Ig heavy chain downstream (3′) of the promoter sequence. Results of gene expression of the various vectors is shown in Table 5, FIG. 2, FIG. 3, and FIG. 4. LSPX1 (SEQ ID NO: 1), LSPX2 (SEQ ID NO: 2), LTP1 (SEQ ID NO: 3), LTP2 (SEQ ID NO: 4), and LTP3 (SEQ ID NO: 5) maintain liver specificity.

TABLE 5

| | | Relative Expression Levels | |
|---|---|---|---|
| Vector designation | Name | HuH7 (liver) | C2C12 (Muscle) |
| pRGNX001 | CAG | +++ | +++ |
| pRGNX002 | TBG | + | − |
| pRGNX003 | hAAT | + | − |
| pRGNX004 | LSPX1 | ++ | − |
| pRGNX005 | LSPX2 | ++ | − |
| pRGNX006 | LTP1 | +++ | − |
| pRGNX007 | LTP2 | + | − |
| pRGNX008 | LTP3 | + | − |

### 6.3. Example 3—Analysis of Gene Expression of Tandem Liver- and Muscle-Specific Promoters

In analogous experiments to Example 2, five GFP-expressing constructs (AAV cis plasmids) having expression cassettes as depicted in FIG. 5, the hAAT promoter cassette depicted in FIG. 1, and an additional control CAG promoter (SEQ ID NO: 17) construct not depicted in the figure and flanked by ITRs, were transfected into HuH7 and C2C12 cells and assessed for gene expression.

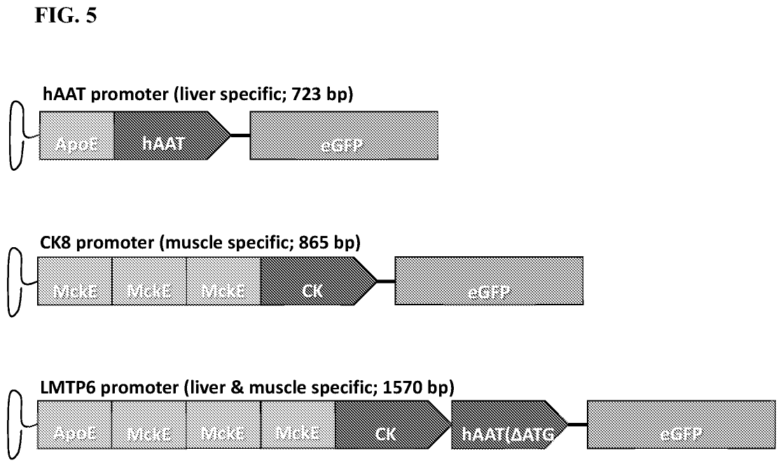

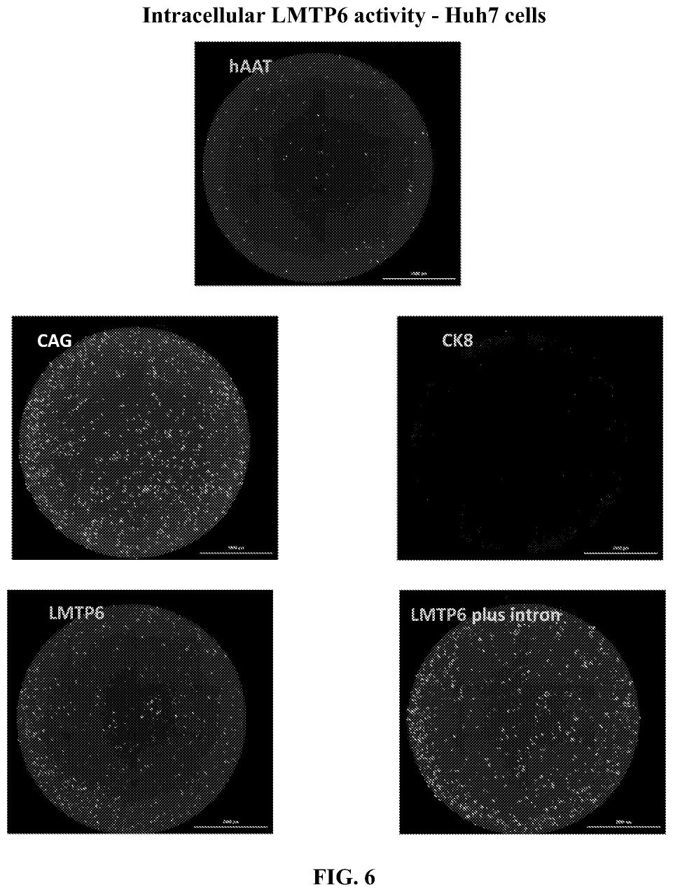

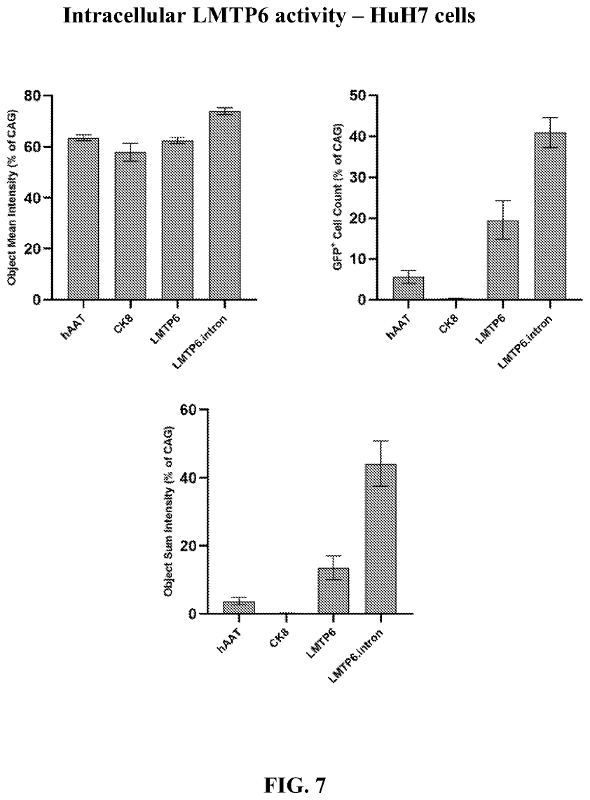

FIG. 6 depicts relative intensity of GFP gene expression for constructs CAG (universal, SEQ ID NO: 17), hAAT (liver-specific, SEQ ID NO: 11), CK8 (muscle-specific, SEQ ID NO: 16), and LMTP6 (dual specificity, SEQ ID NO: 6). The LMTP6 construct with the chimeric intron (LMTP6 plus intron) shows increased GFP gene expression compared to the LMTP6 without the intron. See also quantitative measures of GFP expression in FIG. 7.

As seen in FIG. 3, all liver-specific promoters are silent for GFP expression in muscle cells, while the control muscle promoters (Spc512, CK8) and LMTP6, the promoter with dual specificity for both muscle and liver are active.

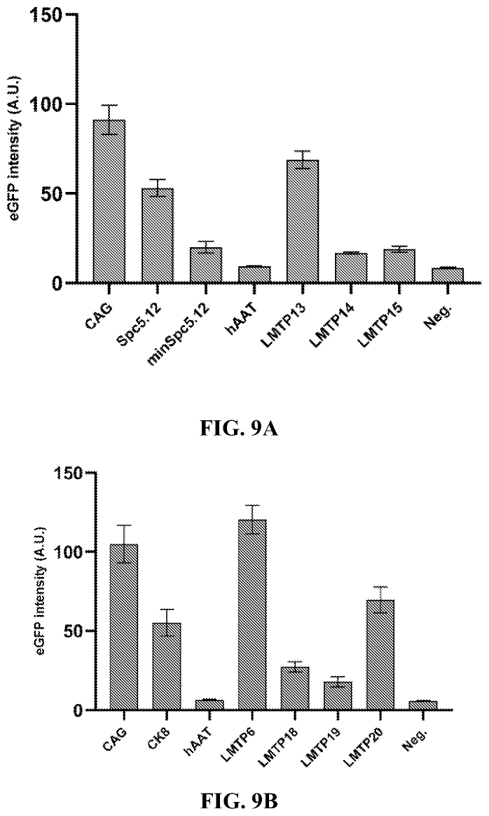

Other experiments, analogous to Example 2, were performed to test LMTP13 (SEQ ID NO: 21), LMTP14 (SEQ ID NO: 22), LMTP15 (SEQ ID NO: 23), LMTP18 (SEQ ID NO: 24), LMTP19 (SEQ ID NO: 25), LMTP20 (SEQ ID NO: 26) (FIG. 8A) and their ability to drive GFP expression in a muscle-cell derived cell line, C2C12 (FIGS. 9A and 9B). Liver/muscle dual specificity promoter LMTP13 shows increased ability to drive GFP expression compared to both the muscle-specific Spc5.12 and minimal Spc5.12 promoter. LMTP14, LMTP15, and the minimal Spc5.12 promoter achieve comparable GFP expression levels and outperform the negative control as well as the liver-specific hAAT promoter. LMTP6 and LMTP20 outperform the muscle-specific CK8 promoter. All LMTP constructs tested show increased GFP expression compared to both the negative control and the liver-specific hAAT promoter.

### 6.4. Example 4—Analysis of Gene Expression of Tandem Liver- and Tandem Liver/Muscle-Specific Promoters Driving Expression of an Anti-Plasma Kallikrein Antibody

A cDNA-based vector was constructed comprising a transgene comprising a nucleotide sequence encoding the heavy and light chain sequences of a pKal antibody (Mab1). The nucleotide sequences encoding the light chain and heavy chain were separated by a Furin-F2A linker (RKRR(GSG)APVKQTLNFDLLKLAGDVESNPGP) or a Furin-T2A linker RKRR(GSG)EGRGSLLTCGDVEENPGP) to create a bicistronic vector. The vector additionally included a constitutive CAG promoter in certain embodiments.

Table 1 above provides the sequences of composite nucleic acid regulatory sequences that may be incorporated into expression cassettes and be operably linked to the transgene to promote liver-specific expression (LSPX1, LSPX2, LTP1, LTP2, or LTP3, SEQ ID NOS: 1-5, respectively), liver and muscle expression (LMTP6, LMTP13, LMTP15, LMTP18, LMTP19 or LMTP20, SEQ ID NOS: 6, 21-26, respectively), liver and bone expression (LBTP1 or LBTP2, SEQ ID NOS: 30-31, respectively) Other promoter sequences provided, include the ApoE.hAAT (SEQ ID NO: 37, Table 1 above) promoter, wherein four copies of the liver-specific apolipoprotein E (ApoE) enhancer were placed upstream of the human alpha 1-antitrypsin (hAAT) promoter.).

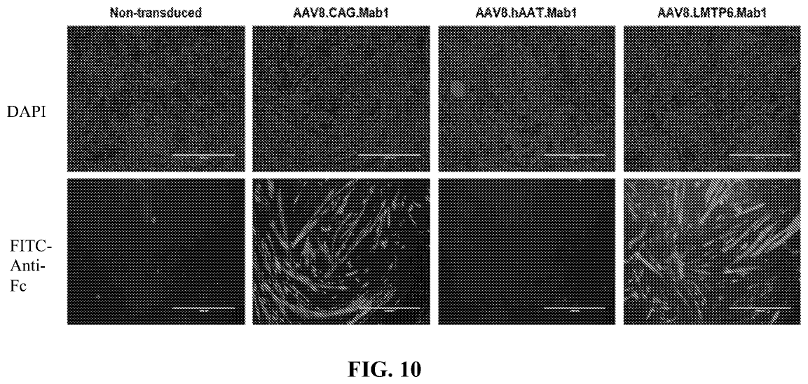

Cis plasmids expressing the pKal Mab1 were packaged in AAV, then rAAV particles evaluated for potency of the transduction by AAV. Each cis plasmid contained Mab1 antibody light chain and heavy chain which are multicistrons driven by the CAG (SEQ ID NO: 17), ApoE.hAAT (SEQ ID NO: 37) or LMTP6 (SEQ ID NO: 6) promoter. Full-length Mab1 antibody light chain and antibody heavy chain genes were separated by a furin 2A linker to ensure separate expression of each antibody chain. The entire cassette is flanked by AAV2 ITRs, and the genome is encapsidated in an AAV8 capsid for delivery to C2C12 cells (1E¹⁰ vg per well). For detection of antibody protein, following transduction, the cells are treated with FITC conjugated anti-Fc (IgG) antibody. The AAV8.CAG.Mab1 and AAV8.LMTP6.Mab1 infected cells show high expression in muscle cells, whereas the AAV8.hAAT.Mab1 infection does not result in expression of the antibody in muscle cells (FIG. 10). Cells appeared to be equally confluent and viable in all test wells, as seen by DAPI (DNA) staining (FIG. 10).

### 6.5. Example 5—Antibody Expression and Vector Biodistribution in Mice Treated with AAV8.Mab1 Vectors Driven by Various Promoters

Thyroxine binding globulin (TBG, SEQ ID NO: 10)) and alpha-1 antitrypsin (hAAT, SEQ ID NO: 11) promoters have been widely used as liver-specific promoters in previous pre-clinical and clinical gene therapy studies. A panel of designed promoter cassettes derived from multiple promoters and enhancers were generated and tested in vitro by transfecting Huh7 cells, a human liver cell line. Promoter candidates were selected, which include ApoE.hAAT (SEQ ID NO: 37), LSPX1 (SEQ ID NO: 1), LSPX2 (SEQ ID NO: 2), LTP1 (SEQ ID NO: 3) and LMTP6 (SEQ ID NO: 6). AAV8 vectors encoding Mab1 regulated by these promoter candidates were then generated. AAV8 vectors encoding Mab1 regulated by CAG and TBG promoters served as controls for ubiquitous and liver-specific promoters, respectfully. Strength of these promoters and vector biodistribution were tested in vivo by measuring Mab1 protein expression compared to vector genome copy in each wild type mouse.

Vectors were administered intravenously to C57Bl/6 mice at equivalent doses (2.5×10¹² vg/kg). Mouse serum was collected biweekly, and Mab1 protein expression levels were determined by ELISA. Liver samples were harvested at 49 days post vector administration. The presence of viral genomes in each sample was quantified using Mab1 probe and primer by Droplet Digital PCR (ddPCR) (the NAICA™ system from Stilla). The genome copy number of glucagon was also measured simultaneously in each sample, the viral genomes were then normalized and demonstrated as vector genome copy number per cell (assuming 2 glucagon/cell). Statistical analysis was performed using one-way ANOVA in GraphPad Prism 8.

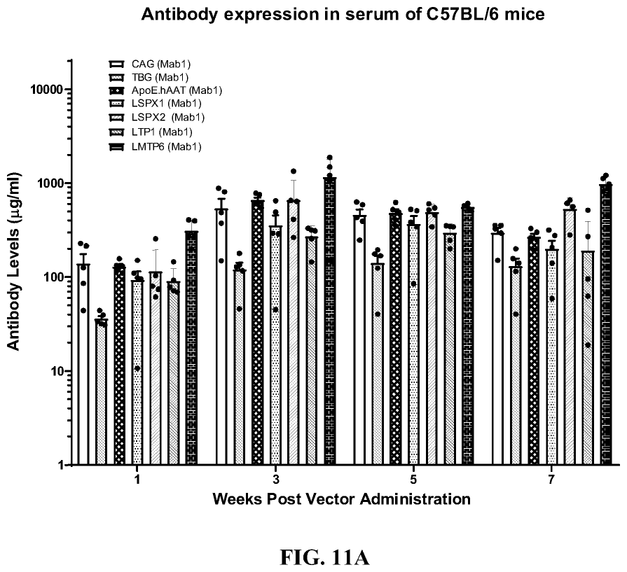

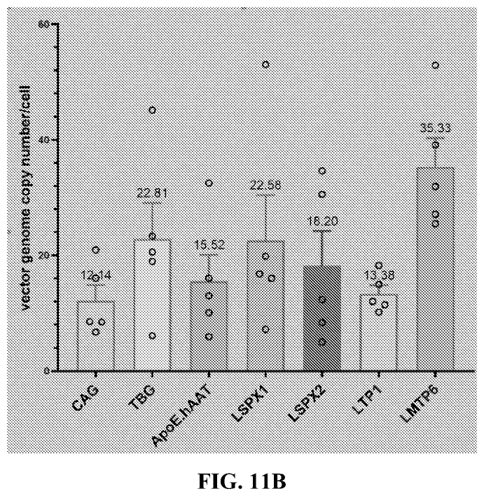

Among the AAV8 vectors with liver-specific promoters, the vectors driven by the ApoE.hAAT (SEQ ID NO: 37) and LMTP6 (SEQ ID NO: 6) promoters provided the highest amount of protein expression at all time points (FIG. 11A). While for the biodistribution data, there was no significant difference of vector genome copy number per cell in liver samples in animals treated with vectors driven by different promoters (FIG. 11B).

All liver-specific promoters outperform the TBG promoter (SEQ ID NO: 10), and the dual-specific LMTP6 promoter (SEQ ID NO: 6) consistently shows the highest expression in the serum (μg/ml) (FIG. 11A).

### 6.6. Example 6: Characterization of Mab1 Expression Regulated by Tissue-Specific Promoters Following Intravenous Administration

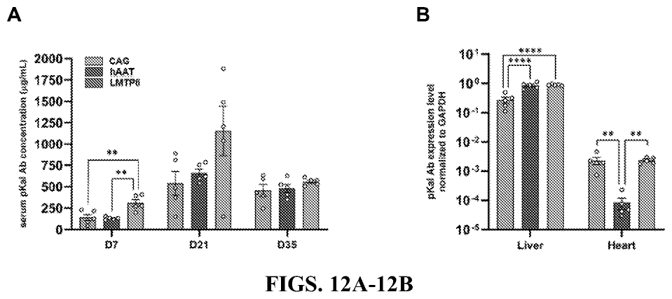

Analogous in vivo experiments (to Example 5) were conducted focusing on studying Mab1 (anti-pKal antibody) expression regulated by the ApoE.hAAT (SEQ ID NO: 37) or LMTP6 (SEQ ID NO: 6) promoters. AAV8 vectors (2.5×10¹² vg/kg) were intravenously administered to adult C57BL/6 mice (N=5/group). Mab1 levels were quantified from mouse serum at various time points with an ELISA. Vectors driven by the LMTP6 promoter displayed significantly increased antibody concentration at Day 7 (FIG. 12A).

In addition, transgene expression in the liver and heart was quantified with ddPCR analysis of Mab1 mRNA copies normalized to GAPDH across tissues. ApoE.hAAT and LMTP6 driven vectors exhibited increased transgene expression compared to CAG in the liver. LMTP6 demonstrated comparable expression to the CAG promoter in cardiac muscle while hAAT activity was far reduced (FIG. 12B).

### 6.7. Example 7: Characterization of Mab1 Expression Regulated by Tissue-Specific Promoters Following Intramuscular Administration

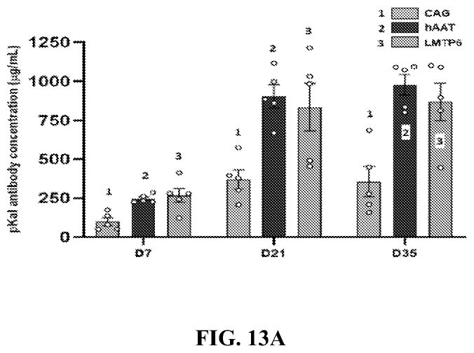

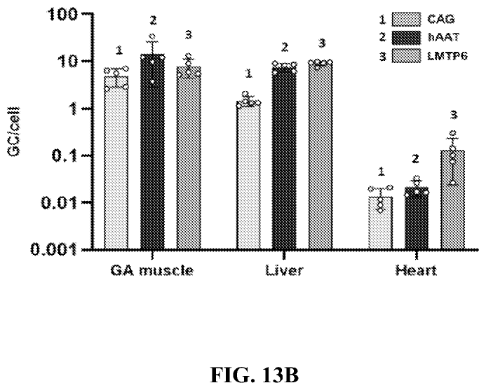

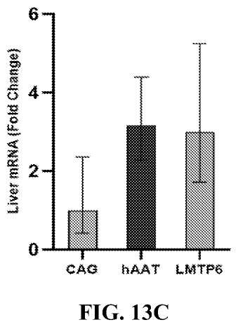

In a previous study, high liver-driven expression of Mab1 with AAV8 regulated by the ApoE.hAAT (SEQ ID NO: 37) or LMTP6 (SEQ ID NO: 6) promoters was identified. The goal of this study was to characterize muscle-driven expression of the LMTP6 promoter following direct injection of Mab1 vectors into the gastrocnemius (GA) muscle. Animals received bilateral injections of 5×10¹⁰ vg into the GA muscle. Serum was collected biweekly to measure systemic Mab1 concentration (FIG. 13A) Animals were harvested at 49 days post-injection, and relevant tissues (liver, GA muscle, heart) were analyzed for vector biodistribution and transgene expression.

Vectors in which gene expression is regulated by the ApoE.hAAT and LMTP6 promoters demonstrated significantly increased antibody concentrations in serum compared to CAG promoter driven expression at all time points (FIG. 13A). The ApoE.hAAT and LMTP6 were not significantly different from each other in this experiment. Vector genome copies per cell of vectorized Mab1 was detected and quantified in GA, liver and heart (FIG. 13B) with a notable difference of higher quantity of genome detected in heart for the dual muscle/liver promoter, LMTP6 vector. Increased liver RNA expression was also detected for all test vectors directly injected into GA muscle at 49 days (relative fold gene expression compared to a reference gene) (FIG. 13C). Gene expression (mRNA μg/mL) data from each of liver, GA muscle, and heart (FIG. 13D) indicates the dual specificity of LMPT6 in liver and muscle tissues following intramuscular administration, whereas the ApoE.hAAT-driven samples were reduced in muscle compared to LMTP6 and CAG. Significant differences were also seen between the ApoE.hAAT and LMTP6 groups.

### 6.8. Example 8—Analysis of Gene Expression of Tandem Liver- and Tandem Liver/Muscle-Specific Promoters Driving Expression of an Enzyme

6.8.1 In Vitro Expression of a Lysosomal Enzyme

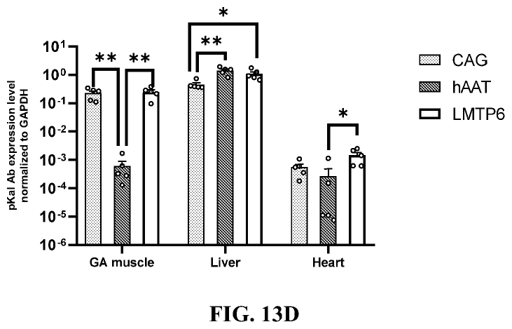

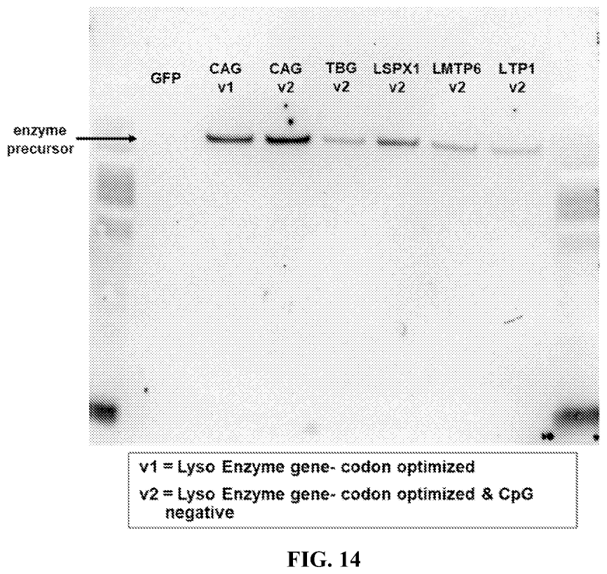

Analogous in vitro experiments (to Example 4) were conducted except Huh7 cells (hepatocytes) were transduced with lysosomal enzyme driven by different promoters and tested for secretion of the protein precursor (FIG. 14).

6.8.2 In Vivo Expression of a Lysosomal Enzyme

An in vivo study was conducted in C57BL/6 mice to evaluate the serum expression and activity of a lysosomal enzyme expressed via AAV delivery to the wild-type mice. On day 0, each animal was administered 5e12 GC/kg body weight (a final dose of 1e11 GC/animal) cis plasmid by IV. For 12 weeks, biweekly collection of serum/plasma from all animals, as such, at week 0, 2, 4, 6, 8, 10, 12 (at necropsy). The animals were perfused at necropsy with PBS to remove blood from organs before sample collection. Serum was collected from each blood sample and analyzed for lysosomal enzyme activity. Tissues were also collected at necropsy for analysis of biodistribution of each sample containing different promoters driving transgene expression in vivo.

TABLE 6
Enzyme activity post-AAV.LysoEnzyme injection

| | Day 28 | Day 42 | Day 56 | Day 70 | Day 84 |
|---|---|---|---|---|---|
| TBG1^{#} | 3.53 ± 0.46 | 4.73 ± 0.80 | 1.92 ± 0.31 | 3.94 ± 0.68 | 3.07 ± 0.99 |
| TBG2^{#} | 3.98 ± 0.57 | 6.42 ± 1.57 | 4.58 ± 1.75 | 11 ± 9.54 | 12.21 ± |
| | | | | | 15.21 |
| LSPX1 | 6.35 ± 2.89 | 6.66 ± 2.05 | 6.87 ± 3.26 | 7.53 ± 2.53 | 15.31 ± |
| | | | | | 11.26 |
| LTP1 | 7.88 ± 1.95 | 7.57 ± 1.48 | 3.91 ± 1.51 | 8.15 ± 1.66 | 7.52 ± 1.42 |
| LMTP6 | 4.84 ± 2.81 | 7.54 ± 3.44 | 5.2 ± 5.18 | 7.41 ± 3.20 | 10.09 ± 8.34 |
| Untreated | 2.52 ± 0.21 | 3.79 ± 0.56 | 1.02 ± 0.86 | 2.81 ± 0.71 | 3.59 ± 0.54 |

^{#}TBG1 = Lysosomal enzyme gene codon optimized; TBG2 = Lysosomal enzyme codon optimized & CpG depleted.

Activity of enzyme in the collected serum was tested, and LSPX1, LTP1 and LMTP6 appear to express adequate enzyme through day 84, having activity better than untreated (endogenous serum enzyme levels) or TBG1-driven constructs.

### 6.9. Example 9—Analysis of Gene Expression of Tandem Liver- and Bone-Specific Promoters

6.9.1 In Vitro Transfection and Expression of GFP

In analogous experiments to Example 2, GFP-expressing constructs (AAV cis plasmids) as depicted in FIG. 8B, including a construct utilizing the universal CAG promoter operably linked to GFP, will be transfected into HuH7 and hFOB 1.19 cells. Briefly, Huh-7 cells and hFOB 1.19 cells will be maintained at 37° C. in a 5% CO2 incubator in Dulbecco's modified Eagle's medium (Corning, Corning, N.Y., USA) supplemented with 10% fetal bovine serum and penicillin-streptomycin (100 U/ml). Transient transfection will be performed with 1 μg of each vector plasmid expressing GFP using Lipofectamine 3000 (Invitrogen, Carlsbad, Calif., USA) in a 6 well plate. After 48 hr transfection will be assessed by fluorescence microscopy and cell pellet and medium will be collected and stored at −20° C. until further processing.

6.9.2. In Vitro Transduction and Expression of Gene of Interest

Cis plasmids expressing antibody, lysosomal enzyme or other gene of interest will be packaged in AAV and rAAV particles will be evaluated for potency of the transduction by AAV. Each cis plasmid contains lysosomal enzyme or other gene of interest, such as an antibody light chain and heavy chain which are multicistrons driven by the same promoter. In the instance of antibody transgenes, full-length antibody light chain and antibody heavy chain genes are separated by a furin 2A linker to ensure separate expression of each antibody chain. The entire cassette is flanked by AAV2 ITRs, and the genome is encapsidated in an AAV8 capsid for delivery to hFOB 1.19 cells. For detection of protein, following transduction, the cells will be treated with FITC conjugated anti-transgene antibody or other detection method. The AAV8.CAG.transgene, AAV8.TBG.transgene, and/or AAV8.hAAT.transgene will be used as controls. Confluency and viability of the cells will be confirmed using DAPI staining.

6.9.3. In Vivo Expression of Vectorized Antibody

Mice will be injected IV with 2.5E12 gc/kg or 1E13 gc/kg rAAV8 vectors encoding a transgene encoding a therapeutic antibody (For Example as in Table 4C or 4D) regulated by different liver-specific, liver-tandem and liver-bone promoters (LBTP1 and LBTP2) and serum expression of the transgene will be evaluated. CAG and TBG promoters will be used as controls. Serum will be collected from the mice at weekly intervals.

### 6.10. Example 10—Analysis of Translation Start Sites from RACE Reaction Products from Genes Driven by Tandem Promoters

6.10.1 Generation of Rapid Amplification of cDNA Ends (RACE) Products

Plasmids containing DNA transgenes (enhanced green fluorescent protein (eGFP)) driven by different promoters were transfected into muscle myoblast cell line (C2C12 cells) or a liver cell line (Huh7 cells). Total RNA was extracted from transfected cells using NucleoSpin RNA kit (Item No. REF 740955; Macherey-Nagel, Germany) 2 to 5 days after transfection. cDNA was synthesized using gene specific primer (SP1, Table 7; SEQ ID NO: 38) and a 2^{nd} Generation RACE kit (Cat. No. 03353621001; Roche) by standard instruction methods. After cDNA synthesis, a homopolymeric A-tail was added to the 3′end of first-strand cDNA using recombinant terminal transferase and dATP. The dA-tailed cDNA was amplified using the Expand High Fidelity PCR System (Millipore Sigma, Cat #11732641001) with SP2 and oligo-dT-anchor primer (Table 7; SEQ ID NO: 39 and SEQ ID NO: 40, respectively). Since multiple variable length products were anticipated, individual bands were not isolated by gel extraction and instead the entirety of products were purified together, then sequenced and analyzed.

TABLE 7
RACE primers

| | |
|---|---|
| SP1 | 5′-CTTCACCTCGGCGCGGGTCTTGTAGTT-3′ |
| | (SEQ ID NO: 38) |
| SP2 | 5′-CTTGTAGTTGCCGTCGTCCTTGAAGAAG-3′ |
| | (SEQ ID NO: 39) |
| Oligo-dT- | 5′-GAC ACG CAT ATC GAT GTC GAC TTT |
| anchor | TTT TTT TTT TTT-TV-3′ |
| primer | (SEQ ID NO: 40) |
| | (V = a mixture of A, C, and G) |

6.10.2. Analysis of RACE Products: Sequencing and Data Processing

Rapid Amplification of cDNA Ends (RACE) reaction products were submitted for amplicon-based sequencing on an ILLUMINA® (San Diego, Calif., USA) platform through a GENEWIZ® Amplicon-EZ system (South Plainfield, N.J., USA).

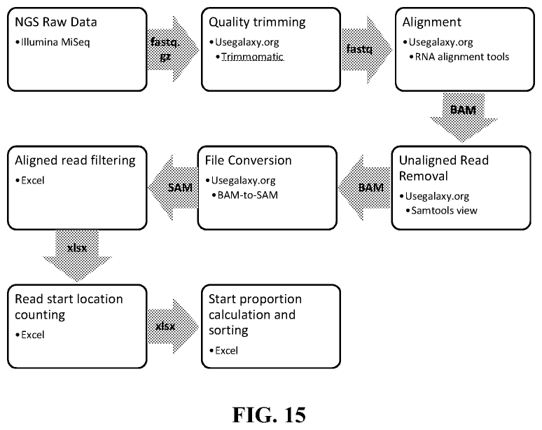

Resultant sequence data of file type fastq.gz were processed via open source data analysis tools through usegalaxy.org (Afgan, et al. “The Galaxy platform for accessible, reproducible and collaborative biomedical analyses: 2018 update”, Nucleic Acids Research, Volume 46, Issue W1, 2 Jul. 2018, Pages W537—W544, doi:10.1093/nar/gky379). See FIG. 15. A “read” refers to an uninterrupted series of nucleotides representing a sequence of the template, in this case each read represents a cDNA copy of the RNA transcripts from the test sample. A measure of the number of RNA transcripts that have the same starting site will indicate the most transcriptionally active sites.

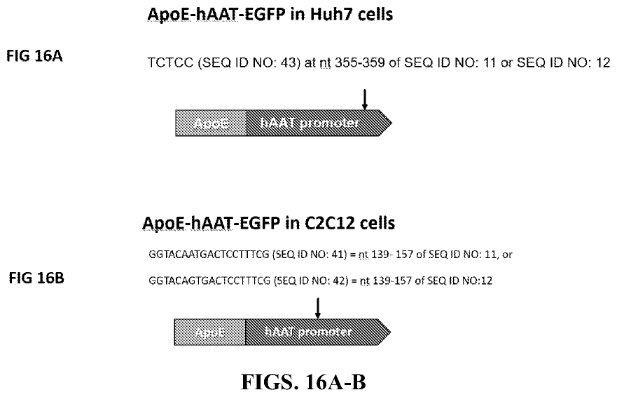

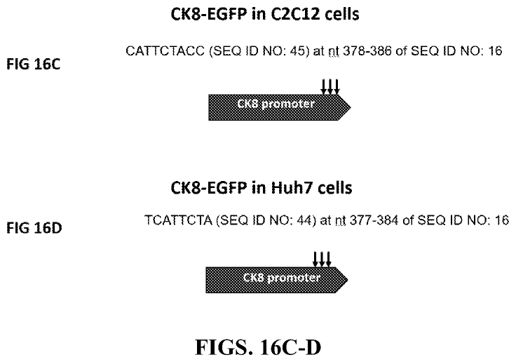

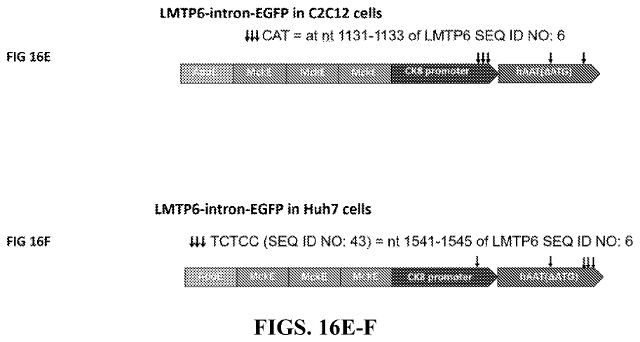

Briefly, low quality base calls on the ends of forward and reverse reads were removed (or “trimmed”) using Trimmomatic (Bolger, A M, et al., “Trimmomatic: a flexible trimmer for Illumina sequence data”, Bioinformatics, Volume 30, Issue 15, 1 Aug. 2014, Pages 2114-2120, doi.org/10.1093/bioinformatics/btu170). Paired-end reads were aligned to reference plasmid sequence using an RNA alignment tool resulting in a BAM file, and another tool, Samtools view, was then implemented to remove unaligned reads from the BAM file (Li, H. et al., 2009, “The Sequence Alignment/Map format and SAMtools.” Bioinformatics, 25 (16), pp. 2078-2079. doi:10.1093/bioinformatics/btp352). In order to continue processing the data, a BAM-to-SAM tool was used to convert the binary BAM file to a tabular SAM file. Subsequent data manipulation steps were performed in Microsoft® Excel (Li, H. et al., 2009, Bioinformatics, supra; Li, H., 2011, “Improving SNP discovery by base alignment quality.” Bioinformatics, 27 (8), pp. 1157-1158, doi:10.1093/bioinformatics/btr076; Li, H., 2011, “A statistical framework for SNP calling, mutation discovery, association mapping and population genetical parameter estimation from sequencing data.” Bioinformatics, 27 (21), pp. 2987-2993, doi:10.1093/bioinformatics/btr509). Reads with MAPQ values below 60 and length less than 20 were removed. The number of instances in which the first base of a read pair aligned with each locus in the reference sequence was tabulated. The region of interest used to calculate transcription start sites was defined as starting from the 5′ end of the 5′ ITR and ending 300 bp 5′ of gene specific primer (GSP). The total number of reads with alignment start sites within the region of interest was tallied and the number of reads starting at each locus within that region was divided by the total and tabulated. Loci which represented greater than or equal to 1% of the total read start count within that region were highlighted as potential transcription start sites. See Tables 8-14 and FIGS. 16A-16F.

6.10.3. Transcription Start Sites (TSS) as Determined by RACE

The data revealed both major and minor transcription start sites (TSS) for each promoter-driven transgene (eGFP) tested in liver and muscle cells, as seen in Tables 8-11. Nucleotide (nt) start numbers were provided (in the Tables) based on the reference plasmid sequence. Once the sequence is identified, the sequence was correlated with individual SEQ ID NOs of promoters described herein.

For the hAAT promoter in C2C12 cells, a major transcription start site, albeit a TSS with low activity, was identified at nucleotides (nt) 660-678 (Table 8, rows 1-6 and 13 highlighted in gray) which accounts collectively for 34% of total filtered reads. Nucleotides 660-678, GGTACAATGACTCCTTTCG (SEQ ID NO: 41), correspond to nucleotides 139-157 of the hAAT promoter sequence SEQ ID NO: 11, or GGTACAGTGACTCCTTTCG (SEQ ID NO: 42) which is at nt 139-157 of SEQ ID NO: 12, a modified ΔATG hAAT sequence. Alternative splicing sites were also observed. For example, alternative splicing sites were also identified by NGS in C2C12 for a gene driven by the hAAT promoter, whereas skipping of bases did occur (nt 919-1052, —678-815, and 680-1052 based on the reference plasmid sequence numbering, data not shown).

TABLE 8
Active start sites (>=1% filtered reads) based on an ApoE-
hAAT promoter-driven transgene expressed in C2C12 cells.

| Start | |
|---|---|
| (nt number per reference | |
| eGFP plasmid sequence) | Read Start/Reads in Range |
| 674 | 10% |
| 673 | 6% |
| 671 | 6% |
| 664 | 5% |
| 660 | 3% |
| 678 | 3% |
| 1055 | 2% |
| 744 | 2% |
| 709 | 2% |
| 688 | 1% |
| 871 | 1% |
| 876 | 1% |
| 670 | 1% |
| 1081 | 1% |
| 1083 | 1% |
| 1082 | 1% |

The major transcription start site of hAAT promoter in Huh7 cells was identified at 876-880, TCTCC (SEQ ID NO: 43)(Table 9, rows 1-2, 5-6 and 10) and accounted for 52% of total filtered reads. The sequence TCTCC (SEQ ID NO: 43) corresponds to nt 355-359 of SEQ ID NO: 11 or SEQ ID NO: 12. Table 9.

TABLE 9
Active start sites (>=1% filtered reads) based on an ApoE-hAAT
promoter-driven transgene expressed in Huh7 cells

| Start | |
|---|---|
| (nt number per Ref plasmid) | Read Start/Reads in Range |
| 876 | 32% |
| 878 | 12% |
| 1081 | 10% |
| 1082 | 6% |
| 877 | 5% |
| 880 | 3% |
| 1080 | 3% |
| 884 | 2% |
| 1055 | 2% |
| 879 | 2% |
| 1083 | 1% |

A TSS of the CK8 promoter, was found at nt 1024-1031 (TCATTCTA SEQ ID NO: 44) (Table 10, rows 1-3 and 6-7), which was highly active in C2C12 cells, producing (collectively) 81% of the reads in the muscle cells. Nucleotides 1024-1031 (TCATTCTA, SEQ ID NO: 44) correspond to nt 377-384 of SEQ ID NO: 16. Table 10.

TABLE 10
Active start sites (>=1% filtered reads) based on a CK8
promoter-driven transgene expressed in C2C12 cells

| Start | |
|---|---|
| (nt number per Ref plasmid) | Read Start/Reads in Range |
| 1028 | 59% |
| 1027 | 17% |
| 1025 | 5% |
| 1033 | 2% |
| 1249 | 2% |
| 1024 | 1% |
| 1031 | 1% |

The same site identified as a TSS in C2C12 cells correlates to the site at 1025-1033 (CATTCTACC, SEQ ID NO: 45)(Table 11, rows 1-3 and 6-7) in Huh7, although the site is less active (62% reads, Table 11) when compared to transcriptional activity of the promoter in C2C12 cells (Table 10). Nucleotides 1025-1033 (CATTCTACC, SEQ ID NO: 45) correspond to nt 378-386 of SEQ ID NO: 16.

TABLE 11
Active start sites (>=1% filtered reads) based on a CK8 promoter-
driven transgene expressed in Huh7 cells

| Start | |
|---|---|
| (nt number per Ref | |
| plasmid) | Read Start/Reads in Range |
| 1028 | 43% |
| 1027 | 12% |
| 1025 | 4% |
| 1222 | 4% |
| 1247 | 2% |
| 1031 | 2% |
| 1033 | 1% |
| 1082 | 1% |
| 1248 | 1% |

Upon testing the LMTP6 promoter in C2C12 cells, three TSSs were identified: 1321-1324 which site correlates with the CK8 starting site (accounting for 33% of total reads), 1505 which correlates with the hAAT promoter in C2C12 cells, and 1702-1737 which correlates with the starting site of hAAT in Huh7 cells. See Table 12.

The active muscle-specific TSS at 1321-1324 (Table 12, rows 1-2 and 5) is found on the CK8 promoter portion of the tandem promoter sequence, and corresponds to nucleotides 1131-1133 (CAT) of SEQ ID NO: 6. Nucleotide 1505, although located in the hAAT portion of the LMTP6 tandem promoter, is slightly active in C2C12 cells and corresponds to nt 1314 of SEQ ID NO: 6. Also, another slightly active TSS identified at 1702-1737 corresponds to nt 1512-1547 of SEQ ID NO: 6.

TABLE 12
Active start sites (>=1% filtered reads): LMTP6 promoter
(+intron) in C2C12 cells

| Start | |
|---|---|
| (nt number per Ref | |
| plasmid) | Read Start/Reads in Range |
| 1324 | 25% |
| 1323 | 6% |
| 1505 | 4% |
| 1704 | 3% |
| 1321 | 2% |
| 1737 | 2% |
| 1702 | 1% |

For the LMTP6 promoter in Huh7 cells, a major starting site is located in the liver-specific hAAT promoter (at 1732-1736)(Table 13, rows 1˜4 and 8) which is highly active corresponding to at least 72% of the total filtered reads. Two minor TTSs were identified as i) a TSS on the hAAT promoter (at 1514-1516), and ii) another TSS on the CK8 promoter (at 1324). See Table 13.

TABLE 13
Active start sites (>=1% filtered reads): LMTP6 promoter
(+intron) in Huh7 cells

| Start | |
|---|---|
| (nt number per Ref plasmid) | Read Start/Reads in Range |
| 1732 | 45% |
| 1734 | 14% |
| 1733 | 8% |
| 1736 | 3% |
| 1515 | 2% |
| 1740 | 2% |
| 1514 | 2% |
| 1735 | 2% |
| 1516 | 2% |
| 1324 | 1% |

The LMTP6 promoter site identified at 1732-1736 and active in Huh7 cells is TCTCC (SEQ ID NO: 43) and corresponds to nt 1541-1545 of SEQ ID NO: 6. The LMTP6 promoter site identified at 1514-1516 and also slightly active in Huh7 cells is CAG and corresponds to nt 1323-1325 of SEQ ID NO: 6, and the TSS identified at 1324 is on the CK8 promoter of the tandem promoter sequence and corresponds to nucleotide 1133 of SEQ ID NO: 6.

TABLE 14
RACE TSS Summary

| Promoter/ | |
|---|---|
| gene tested | |
| (cells) | TSS Conclusions |
| ApoE-hAAT-driven | A highly active TSS of the hAAT promoter in Huh 7 cells: |
| eGFP transgene | TCTCC (SEQ ID NO: 43) = nt 355-359 of SEQ ID NO: 11 or SEQ ID |
| (in Huh7 cells) | NO: 12 |
| ApoE-hAAT-driven | A mildly active TSS of the hAAT promoter in C2C12: |
| eGFP transgene | GGTACAATGACTCCTTTCG (SEQ ID NO: 41) = nt 139-157 of |
| (in C2C12 cells) | SEQ ID NO: 11, or |
| | GGTACAGTGACTCCTTTCG (SEQ ID NO: 42) = nt 139-157 of |
| | SEQ ID NO: 12, having a modified ΔATG hAAT sequence. |
| | Three splicing sites were identified in hAAT. |
| CK8-driven eGFP | An active TSS of the CK8 promoter was found in both C2C12 and |
| transgene | Huh7 cells, yet the site was determined to be more active (more reads) |
| (C2C12 and | when driving expression in C2C12 cells: |
| Huh7 cells) | TCATTCTACC (SEQ ID NO: 46) = nt 377-386 of SEQ ID NO: 16. |
| LMTP6 (+intron)- | CAT = nucleotides 1131-1133 of LMTP6 SEQ ID NO: 6, in the CK8 |
| driven eGFP | promoter portion of the tandem promoter sequence. This sequence |
| transgene | overlaps with the major TSS identified in CK8 (starting at nt 377 of |
| (in C2C12 cells) | SEQ ID NO: 16). |
| | Three transcription start sites and several alternative splicing sites |
| | have been identified, which correlated that observed of CK8 and hAAT |
| | promoter in C2C12 cells. |
| LMTP6 (+intron)- | One major transcription start site TCTCC (SEQ ID NO: 43) = nt 1541- |
| driven eGFP | 1545 of LMTP6 SEQ ID NO: 6. This site overlaps with the active TTS |
| transgene | found in hAAT (= nt 355-359 of SEQ ID NO: 11 or SEQ ID NO: 12). |
| (in Huh7 cells) | One minor TSS (the sequence CAG) has been identified on the hAAT |
| | portion of the tandem promoter at nt 1323-1325 of SEQ ID NO: 6 and |
| | overlaps with GGTACAGTGACTCCTTTCG (SEQ ID NO: 42) which |
| | is at nt 139-157 of SEQ ID NO: 12. |
| | One minor TSS was identified on CK8 portion of the tandem promoter |
| | is at nt 1133 of SEQ ID NO: 6. |
| | Several alternative splicing sites were identified which correlated |
| | with the observation in C2C12 cells for the hAAT promoter. |

Major and minor TSSs were identified for each promoter depending on the cell type in which the gene was expressed. For example, an ApoE-hAAT promoter-driven gene in C2C12 (muscle) cells contained several start sites with very low activity, while the same plasmid expressed in Huh7 (liver) cells reveals high activity at a different TSS. Tables 10 and Table 11. CK8 promoter behaved similarly in C2C12 cells and Huh7 with respect to TSS, however the promoter was more active in C2C12, the muscle cell line, as expected.

Interestingly, the tandem promoter, LMTP6, resulted in a major TSS at nt 1541-1545 of LMTP6 SEQ ID NO: 6 in liver cells, which correlates to the same TSS for the single promoter hAAT. A minor TSS correlates between both the tandem LMTP6 and hAAT alone, while the correlative CK8 TSS was considered minor (at nt 1324, only 1% of reads). An LMTP6—driven transgene also correlated with transcriptional sites in C2C12 cells compared to CK8 single promoter. The muscle-specific TSS found active with LMTP6 in C2C12 cells overlaps with the major TSS identified in CK8 in these cells. Given the integrity of transcriptional start sites shown herein, and by eliminating the extra ATG translational start site in tandem promoters which further reduces overall translation of aberrant species of final protein, the tandem promoters of the disclosure provide for efficient and robust expression of transgenes.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A recombinant expression cassette comprising a composite nucleic acid regulatory element comprising a) one or two copies of Mic/BiKE arranged in tandem, one or two copies of ApoE enhancer arranged in tandem, or one or two copies of MckE arranged in tandem, and b) at least two promoters arranged in tandem wherein at least one promoter is hAAT, wherein the hAAT is start-codon modified (ΔATG), operably linked to a transgene.

2. The recombinant expression cassette of claim 1 comprising a TBG promoter, a CK8 promoter, an Spc5.12 promoter, an Sp7/Osx promoter or a minSp7/Osx promoter.

3. The recombinant expression cassette of claim 1, wherein the nucleic acid regulatory element is LTP1, LTP2, LTP3, LMTP6, LMTP13, LMTP14, LMTP15, LMTP18, LMTP19, LMTP20, LBTP1, or LBTP2.

4. A recombinant expression cassette comprising a composite regulatory element comprising LSPX1 (SEQ ID NO: 1) or LSPX2 (SEQ ID NO: 2) operably linked to a transgene.

5. The recombinant expression cassette of claim 1, where the transgene is a gene or nucleic acid encoding alpha-L-iduronidase (IDUA), iduronate-2-sulfatase (IDS), CLN1, CLN2, CLN10, CLN13, CLN5, CLN11, CLN4, CLN14, CLN3, CLN6, CLN7, CLN8, CLN12, N-sulfoglucosamine sulfohydrolase (SGSH), N-acetyl-alpha-D-glucosaminidase (NAGLU), arylsulfatase B, GLANS, GLB1, COL1A1, COL1A2, IFITM5, SERPINF1, CRTAP, LEPRE1, P3H1, PPIB, Glucocerebrosidase (GBA1), dopamine decarboxylase, acid maltase, GAA, aryl sulfatase A, beta-glucuronidase, glucosamine-6-sulfate sulfatase, hyaluronidase, sphingomyelinase, npc1, alpha subunit of beta-hexosaminidase, beta subunit of beta-hexosaminidase, alpha-galactosidase, Fucosidase (FUCA1 gene), alpha-mannosidase, Beta-mannosidase, cholesterol ester hydrolase, Neurturin, glial derived growth factor (GDGF), tyrosine hydroxylase, glutamic acid decarboxylase, fibroblast growth factor-2 (FGF-2), brain derived growth factor (BDGF), neuraminidase, betagalactosidase, SMN, Frataxin, SOD1, Glucose-6-phosphatase, MTM1, UGT1A1, CASQ2, MECP2, CNGB3, CNGA3, GNAT2, PDE6C, CDM, LAMP2, CFTR, mini-Dystrophin, micro-dystrophin, human-alpha-sarcoglycan, SERCA2a, TNFR:Fc Fusion Gene, GAA, gamma-sarcoglycan, hMERTK, sFLT01, huFollistatin344, GDNF, cuARSA, anti-HCV shRNA, hSGCA, PG9DP, PBGD, P1ND4v2, alphalAT, hGAA, RS1, hCHM, JeT-GAN, micro-Dystrophin, hRS1, hAQP1, Factor IX, hLDLR, rAAVrh74.MHCK7.DYSF.DV, ZFP nuclease, NF-kB.IFN-β, CLN6, hSGSH, 5IL-1Ra, CNGA3, CNGB3, OTC, Factor VIII, ZFP nuclease, anti-VEGF, RPGR, hARSB, ND4, MTM1, UGT1A1, CNGB3, hPDE6B, RPGR, hNAGLU, GALGT2, TNFR:Fc Fusion Gene, Neurturin, NGF, tgAAC09, LPL, Neurturin, hAAT, hRPE65v2, CLN2, GAD, N-sulfoglucosamine sulfohydrolase (SGSH), SERC2a, CMV.huFollistatin344, hAADC-2, REP1, CEA, MUC1-peptide-DC-CTL, P1ND4v2, hAADC, Factor IX, AADC, GS010, SMN, B-Domain Deleted Factor VIII, IDS, CLN3, hSGCB, APOE2, hMERKTK, RLBP1, Anti-VEGF antibody, solanezumab, GSK933776, lecanemab, AL-001, ABBV-8E12, UCB-0107, NI-105 (BIIB076), VX15/2503, prasinezumab, NI-202 (BIIB054), MED-1341, NI-204, eptinezumab, fremanezumab, galcanezumab, sevacizumab, LKA-651, solanezumab, GSK933776, lecanemab, ascrinvacumab, tesidolumab, ravulizumab carotuximab, ANX-007, adalimumab, infliximab, golimumab, elezanumab, NI-301, PRX-004, pamrevlumab, Satralizumab, Sarilumab, Tocilizumab, siltuximab, clazakizumab, sirukumab, olokizumab, gerilimzumab, inebilizumab, etrolizumab, romosozumab, ravulizumab, Satralizumab, Sarilumab, Tocilizumab, siltuximab, clazakizumab, sirukumab, olokizumab, gerilimzumab, omolizumab, tezelipumab, benralizumab, reslizumab, tralokinumab, nemolizumab, Aducanumab, crenezumab, gantenerumab, anti-TAU, erenumab, ixekizumab, secukinumab, mepolizumab, ustekinumab, dupilumab, vedolizumab, Natalizumab (anti-integrin alpha 4), alirocumab, evolucomab, evinacumab, E06-scFv, denosumab, nivolumab, pembrolizumab, belimumab, ranibizumab, bevacizumab, brolucizumab, lampalizumab, andecaliximab, adalimumab, infliximab, eculizumab or lanadelumab.

6. The recombinant expression cassette of claim 1, wherein the transgene encodes a therapeutic antibody, or antigen binding fragment thereof.

7. The recombinant expression cassette of claim 1, wherein the composite nucleic acid regulatory element comprises a nucleic acid sequence of SEQ ID NO: 3, SEQ ID NO: 4, SEQ ID NO: 5, SEQ ID NO: 6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31.

8. A vector comprising the expression cassette of claim 1.

9. (canceled)

10. The vector of claim 8 wherein the cassette is suitable for packaging in an AAV capsid.

11. The vector of claim 10, comprising an artificial genome comprising (1) AAV inverted terminal repeats (ITRs) flanking the expression cassette; (2) an expression cassette comprising (a) a composite nucleic acid regulatory control element comprising a nucleic acid sequence of SEQ ID NO: 1, SEQ ID NO: 2, SEQ ID NO: 3, SEQ ID NO: 4, SEQ ID NO: 5, SEQ ID NO: 6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31, b) a poly A signal, c) optionally an intron; and d) a transgene coding for one or more RNA or protein products to which the composite nucleic acid regulatory element is operably linked.

12. An rAAV particle comprising the vector of claim 11, and a capsid protein from an AAV capsid serotype selected from AAV1, AAV1, AAV2, rAAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

13-18. (canceled)

19. A method for treating a disease or disorder in a subject in need thereof comprising the administration of recombinant AAV particles comprising an expression cassette having more than one Mic/BiK enhancer sequences, or ApoE enhancer sequences, or Mck enhancer sequences upstream of one or more liver-specific promoters, wherein at least one liver-specific promoter comprises a modified start codon (ΔATG), operably linked to a transgene.

20. The method of claim 19, wherein the expression cassette comprises nucleic acid sequences set forth in SEQ ID NO: 1, SEQ ID NO: 2, SEQ ID NO: 3, SEQ ID NO: 4, SEQ ID NO: 5, SEQ ID NO: 6, SEQ ID NO: 21, SEQ ID NO: 22, SEQ ID NO: 23, SEQ ID NO: 24, SEQ ID NO: 25, SEQ ID NO: 26, SEQ ID NO: 30, or SEQ ID NO: 31.

21. The method of claim 19, wherein the transgene is selected from alpha-L-iduronidase (IDUA), iduronate-2-sulfatase (IDS), CLN1, CLN2, CLN10, CLN13, CLN5, CLN11, CLN4, CLN14, CLN3, CLN6, CLN7, CLN8, CLN12, N-sulfoglucosamine sulfohydrolase (SGSH), N-acetyl-alpha-D-glucosaminidase (NAGLU), arylsulfatase B, GLANS, GLB1, COL1A1, COL1A2, IFITM5, SERPINF1, CRTAP, LEPRE1, P3H1, PPIB, Glucocerebrosidase (GBA1), dopamine decarboxylase, acid maltase, GAA, aryl sulfatase A, beta-glucuronidase, glucosamine-6-sulfate sulfatase, hyaluronidase, sphingomyelinase, npc1, alpha subunit of beta-hexosaminidase, beta subunit of beta-hexosaminidase, alpha-galactosidase, Fucosidase (FUCA1 gene), alpha-mannosidase, Beta-mannosidase, cholesterol ester hydrolase, Neurturin, glial derived growth factor (GDGF), tyrosine hydroxylase, glutamic acid decarboxylase, fibroblast growth factor-2 (FGF-2), brain derived growth factor (BDGF), neuraminidase, betagalactosidase, SMN, Frataxin, SOD1, Glucose-6-phosphatase, MTM1, UGT1A1, CASQ2, MECP2, CNGB3, CNGA3, GNAT2, PDE6C, CDM, LAMP2, CFTR, mini-Dystrophin, micro-dystrophin, human-alpha-sarcoglycan, SERCA2a, TNFR:Fc Fusion Gene, GAA, gamma-sarcoglycan, hMERTK, sFLT01, huFollistatin344, GDNF, cuARSA, anti-HCV shRNA, hSGCA, PG9DP, PBGD, P1ND4v2, alphalAT, hGAA, RS1, hCHM, JeT-GAN, micro-Dystrophin, hRS1, hAQP1, Factor IX, hLDLR, rAAVrh74.MHCK7.DYSF.DV, ZFP nuclease, NF-kB.IFN-β, CLN6, hSGSH, 5IL-1Ra, CNGA3, CNGB3, OTC, Factor VIII, ZFP nuclease, anti-VEGF, RPGR, hARSB, ND4, MTM1, UGT1A1, CNGB3, hPDE6B, RPGR, hNAGLU, GALGT2, TNFR:Fc Fusion Gene, Neurturin, NGF, tgAAC09, LPL, Neurturin, hAAT, hRPE65v2, CLN2, GAD, N-sulfoglucosamine sulfohydrolase (SGSH), SERC2a, CMV.huFollistatin344, hAADC-2, REP1, CEA, MUC1-peptide-DC-CTL, P1ND4v2, hAADC, Factor IX, AADC, GS010, SMN, B-Domain Deleted Factor VIII, IDS, CLN3, hSGCB, APOE2, hMERKTK, RLBP1, Anti-VEGF antibody, solanezumab, GSK933776, lecanemab, AL-001, ABBV-8E12, UCB-0107, NI-105 (BIIB076), VX15/2503, prasinezumab, NI-202 (BIIB054), MED-1341, NI-204, eptinezumab, fremanezumab, galcanezumab, sevacizumab, LKA-651, solanezumab, GSK933776, lecanemab, ascrinvacumab, tesidolumab, ravulizumab carotuximab, ANX-007, adalimumab, infliximab, golimumab, elezanumab, NI-301, PRX-004, pamrevlumab, Satralizumab, Sarilumab, Tocilizumab, siltuximab, clazakizumab, sirukumab, olokizumab, gerilimzumab, inebilizumab, etrolizumab, romosozumab, ravulizumab, Satralizumab, Sarilumab, Tocilizumab, siltuximab, clazakizumab, sirukumab, olokizumab, gerilimzumab, omolizumab, tezelipumab, benralizumab, reslizumab, tralokinumab, nemolizumab, Aducanumab, crenezumab, gantenerumab, anti-TAU, erenumab, ixekizumab, secukinumab, mepolizumab, ustekinumab, dupilumab, vedolizumab, Natalizumab (anti-integrin alpha 4), alirocumab, evolucomab, evinacumab, E06-scFv, denosumab, nivolumab, pembrolizumab, belimumab, ranibizumab, bevacizumab, brolucizumab, lampalizumab, andecaliximab, adalimumab, infliximab, eculizumab or lanadelumab.

22. The method of claim 21, wherein the transgene encodes a therapeutic antibody, or antigen binding fragment thereof.

23. The method of claim 19, wherein the rAAV is administered intravenously or intramuscularly.

24. A method of producing recombinant AAVs comprising:
(a) culturing a host cell containing:
(i) an artificial genome comprising the recombinant expression cassette of claim 1 flanked by AAV ITRs wherein the recombinant expression cassette is operably linked to a transgene coding for one or more RNA or protein products;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
(iii) sufficient adenovirus helper functions to permit replication and packaging of the artificial genome by the AAV capsid protein; and
(b) recovering recombinant AAV encapsidating the artificial genome from the cell culture.

25. The method of claim 24, wherein the composite nucleic acid regulatory element is LTP1, LTP2, LTP3, LMTP6, LMTP13, LMTP14, LMTP15, LMTP18, LMTP19, LMTP20, LBTP1, or LBTP2.

26. A host cell comprising a plasmid comprising the recombinant expression cassette of claim 1 flanked by AAV ITRs wherein the recombinant expression cassette is operably linked to a transgene coding for one or more RNA or protein products.

27. The host cell of claim 26, wherein the composite nucleic acid regulatory element is LTP1, LTP2, LTP3, LMTP6, LMTP13, LMTP14, LMTP15, LMTP18, LMTP19, LMTP20, LBTP1, or LBTP2.

28. The method of claim 19 wherein the nucleic acid expression cassette comprises a nucleic acid regulatory element of LTP1, LTP2, LTP3, LMTP6, LMTP13, LMTP14, LMTP15, LMTP18, LMTP19, LMTP20, LBTP1, or LBTP2.


SEQUENCE LISTING (50 sequences)

SEQ ID NO: 1 - DNA, 969 bp, Artificial Sequence
  source: /note=“Description of Artificial Sequence Synthetic polynucleotide”
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 2 - DNA, 1050 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 3 - DNA, 1089 bp, Artificial Sequence
  source: /note=“Description of Artificial Sequence Synthetic polynucleotide”
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 4 - DNA, 1430 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 5 - DNA, 1318 bp, Artificial Sequence
  source: /note=“Description of Artificial Sequence Synthetic polynucleotide”
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 6 - DNA, 1579 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 7 - DNA, 100 bp, Artificial Sequence
  source: /note=“Description of Artificial Sequence Synthetic polynucleotide”
aggttaatttttaaaaagcagtcaaaagtccaagtggcccttggcagcatttactctctctgtttgctctggttaataatctcaggagcacaaacattcc

SEQ ID NO: 8 - DNA, 200 bp, Artificial Sequence
  source: /note=“Description of Artificial Sequence Synthetic polynucleotide”
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 9 - DNA, 321 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 10 - DNA, 460 bp, Artificial Sequence
  source: /note=“Description of Artificial Sequence Synthetic polynucleotide”
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 11 - DNA, 393 bp, Artificial Sequence
  source: /note=“Description of Artificial Sequence Synthetic polynucleotide”
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 12 - DNA, 393 bp, Artificial Sequence
  source: /note=“Description of Artificial Sequence Synthetic polynucleotide”
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 13 - DNA, 144 bp, Artificial Sequence
  source: /note=“Description of Artificial Sequence Synthetic polynucleotide”
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 14 - DNA, 288 bp, Artificial Sequence
  source: /note=“Description of Artificial Sequence Synthetic polynucleotide”
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 15 - DNA, 432 bp, Artificial Sequence
  source: /note=“Description of Artificial Sequence Synthetic polynucleotide”
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 16 - DNA, 433 bp, Artificial Sequence
  source: /note=“Description of Artificial Sequence Synthetic polynucleotide”
ccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 17 - DNA, 1676 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 18 - DNA, 133 bp, Artificial Sequence
  source: /note=“Description of Artificial Sequence Synthetic polynucleotide”
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 19 - DNA, 82 bp, Artificial Sequence
  source: /note=“Description of Artificial Sequence Synthetic oligonucleotide”
gtgagtatctcagggatccagacatggggatatgggaggtgcctctgatcccagggctcactgtgggtctctctgttcacag

SEQ ID NO: 20 - DNA, 554 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 21 - DNA, 1080 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 22 - DNA, 568 bp, Artificial Sequence
  source: /note=“Description of Artificial Sequence Synthetic polynucleotide”
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 23 - DNA, 895 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 24 - DNA, 1291 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 25 - DNA, 1147 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 26 - DNA, 1490 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 27 - DNA, 199 bp, Artificial Sequence
  source: /note=“Description of Artificial Sequence Synthetic polynucleotide”
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 28 - DNA, 328 bp, Artificial Sequence
  source: /note=“Description of Artificial Sequence Synthetic polynucleotide”
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 29 - DNA, 174 bp, Artificial Sequence
  source: /note=“Description of Artificial Sequence Synthetic polynucleotide”
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaag

SEQ ID NO: 30 - DNA, 969 bp, Artificial Sequence; identical to SEQ ID NO: 1
  source: /note=“Description of Artificial Sequence Synthetic polynucleotide”

SEQ ID NO: 31 - DNA, 2074 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcacacatacacgaacacacatacatatacatacattcacatatatgcacacacatacacatatacacgcatacacgtacacacaaatgcacactcacacatgcacacacatacataatatacacactctcacacatgcacatacacacatacatacacatacatgtgcatgcacacacacaaatacacatgcatacatccacattcacacagatgcagacacaaatgcacacacacacacacacacacacacacacacacacacacacacgcacactgccaccctgaactagtggtggctaaatgaacaataagtctccatcaccagcttggggggaggtaggtggtagtgtaggtgcccccattgtgtgatcatgttcattgtatgagtttgtctgtgttcattcatcatagtgacagtccccatgtgggtagcagagagtacgtgtgcatgcatcatctccgtgtttgctcatgagtgtgtatgtcagtgtgttccagtctttctgtgtgagtgtcgtccccaatcccccatccccccccccagatctctaattagtggtttggggtttgttccttttccctcctgttcctttcctcagcagcgcggcagcagcggcggcagcctcggtggtagcagcagcagcagcagcagcagcagcagcagcagcagcagcagcagcaacagaagctgccgcgccgctgagtagcagcaaggactccgagtcaagagtaggattgtaggattggatctgagtgggaacaagagtgagctggcctgagagaggagcagatgcctcccagcgccctcaggccacccattgccagtaatcttcaagccagacctcttgagaggagacgggacagccaaccctagcctacccaggtacagacactgggcagttctgggggactgcccacagatgcctattggattcctggggtatgtaggactcccgggtctaccagcccttttcacctttccccatagcacccccaaggaagctctgacaacttgcccatattcctgtttcccacccgtcccctgggcacccccttttcttctctccctcccagatcccttctttggggagctcagcaaatggagcaggaaatttggaccctctgcctccctctctcgccttcctcattggatccggagtcttctccgctgggaaagctgtaattagagggtggatccctacagacagagagcagcccccccacccccaccccccagtcccttctaactttagatctcttctctcccattctcccattctccctccctctcccttctccctctcccactggctcctcggttctctccatctgcctgactccttgggacccggtccccagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 32 - DNA, 1360 bp, Artificial Sequence
  source: /note=“Description of Artificial Sequence Synthetic polynucleotide”
cacacatacacgaacacacatacatatacatacattcacatatatgcacacacatacacatatacacgcatacacgtacacacaaatgcacactcacacatgcacacacatacataatatacacactctcacacatgcacatacacacatacatacacatacatgtgcatgcacacacacaaatacacatgcatacatccacattcacacagatgcagacacaaatgcacacacacacacacacacacacacacacacacacacacacacgcacactgccaccctgaactagtggtggctaaatgaacaataagtctccatcaccagcttggggggaggtaggtggtagtgtaggtgcccccattgtgtgatcatgttcattgtatgagtttgtctgtgttcattcatcatagtgacagtccccatgtgggtagcagagagtacgtgtgcatgcatcatctccgtgtttgctcatgagtgtgtatgtcagtgtgttccagtctttctgtgtgagtgtcgtccccaatcccccatccccccccccagatctctaattagtggtttggggtttgttccttttccctcctgttcctttcctcagcagcgcggcagcagcggcggcagcctcggtggtagcagcagcagcagcagcagcagcagcagcagcagcagcagcagcagcaacagaagctgccgcgccgctgagtagcagcaaggactccgagtcaagagtaggattgtaggattggatctgagtgggaacaagagtgagctggcctgagagaggagcagatgcctcccagcgccctcaggccacccattgccagtaatcttcaagccagacctcttgagaggagacgggacagccaaccctagcctacccaggtacagacactgggcagttctgggggactgcccacagatgcctattggattcctggggtatgtaggactcccgggtctaccagcccttttcacctttccccatagcacccccaaggaagctctgacaacttgcccatattcctgtttcccacccgtcccctgggcacccccttttcttctctccctcccagatcccttctttggggagctcagcaaatggagcaggaaatttggaccctctgcctccctctctcgccttcctcattggatccggagtcttctccgctgggaaagctgtaattagagggtggatccctacagacagagagcagcccccccacccccaccccccagtcccttctaactttagatctcttctctcccattctcccattctccctccctctcccttctccctctcccactggctcctcggttctctccatctgcctgactccttgggacccggtcccca

SEQ ID NO: 33 - DNA, 756 bp, Artificial Sequence
  source: /note=“Description of Artificial Sequence Synthetic polynucleotide”
cgcggcagcagcggcggcagcctcggtggtagcagcagcagcagcagcagcagcagcagcagcagcagcagcagcagcaacagaagctgccgcgccgctgagtagcagcaaggactccgagtcaagagtaggattgtaggattggatctgagtgggaacaagagtgagctggcctgagagaggagcagatgcctcccagcgccctcaggccacccattgccagtaatcttcaagccagacctcttgagaggagacgggacagccaaccctagcctacccaggtacagacactgggcagttctgggggactgcccacagatgcctattggattcctggggtatgtaggactcccgggtctaccagcccttttcacctttccccatagcacccccaaggaagctctgacaacttgcccatattcctgtttcccacccgtcccctgggcacccccttttcttctctccctcccagatcccttctttggggagctcagcaaatggagcaggaaatttggaccctctgcctccctctctcgccttcctcattggatccggagtcttctccgctgggaaagctgtaattagagggtggatccctacagacagagagcagcccccccacccccaccccccagtcccttctaactttagatctcttctctcccattctcccattctccctccctctcccttctccctctcccactggctcctcggttctctccatctgcctgactccttgggacccggtcccca

SEQ ID NO: 34 - DNA, 97 bp, Artificial Sequence
  source: /note=“Description of Artificial Sequence Synthetic oligonucleotide”
gtaagtttagtctttttgtcttttatttcaggtcccggatccggtggtggtgcaaatcaaagaactgctcctcagtggatgttgcctttacttctag

SEQ ID NO: 35 - DNA, 55 bp, Artificial Sequence
  source: /note=“Description of Artificial Sequence Synthetic oligonucleotide”
ataaaggaaatttattttcattgcaatagtgtgttggaattttttgtgtctctca

SEQ ID NO: 36 - DNA, 127 bp, Artificial Sequence
  source: /note=“Description of Artificial Sequence Synthetic polynucleotide”
gatctttttccctctgccaaaaattatggggacatcatgaagccccttgagcatctgacttctggctaataaaggaaatttattttcattgcaatagtgtgttggaattttttgtgtctctcactcg

SEQ ID NO: 37 - DNA, 723 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 38 - DNA, 27 bp, Artificial Sequence
  source: /note=“Description of Artificial Sequence Synthetic primer”
cttcacctcggcgcgggtcttgtagtt

SEQ ID NO: 39 - DNA, 28 bp, Artificial Sequence
  source: /note=“Description of Artificial Sequence Synthetic primer”
cttgtagttgccgtcgtccttgaagaag

SEQ ID NO: 40 - DNA, 38 bp, Artificial Sequence
  source: /note=“Description of Artificial Sequence Synthetic primer”
gacacgcatatcgatgtcgacttttttttttttttttv

SEQ ID NO: 41 - DNA, 19 bp, Artificial Sequence
  source: /note=“Description of Artificial Sequence Synthetic oligonucleotide”
ggtacaatgactcctttcg

SEQ ID NO: 42 - DNA, 19 bp, Artificial Sequence
  source: /note=“Description of Artificial Sequence Synthetic oligonucleotide”
ggtacagtgactcctttcg

SEQ ID NO: 43 - DNA, 5 bp, Artificial Sequence
  source: /note=“Description of Artificial Sequence Synthetic oligonucleotide”
tctcc

SEQ ID NO: 44 - DNA, 8 bp, Artificial Sequence
  source: /note=“Description of Artificial Sequence Synthetic oligonucleotide”
tcattcta

SEQ ID NO: 45 - DNA, 9 bp, Artificial Sequence
  source: /note=“Description of Artificial Sequence Synthetic oligonucleotide”
cattctacc

SEQ ID NO: 46 - DNA, 10 bp, Artificial Sequence
  source: /note=“Description of Artificial Sequence Synthetic oligonucleotide”
tcattctacc

SEQ ID NO: 47 - protein, 7 aa, Artificial Sequence
  source: /note=“Description of Artificial Sequence Synthetic peptide”
LGETTRP

SEQ ID NO: 48 - protein, 9 aa, Artificial Sequence
  source: /note=“Description of Artificial Sequence Synthetic peptide”
LALGETTRP

SEQ ID NO: 49 - protein, 31 aa, Artificial Sequence
  source: /note=“Description of Artificial Sequence Synthetic polypeptide”
  VARIANT (5)..(7): /replace=“ ”
  SITE (5)..(7): /note=“This region may or may not be present in its entirety”
  SITE (1)..(31): /note=“Variant residues given in the sequence have no preference with respect to those in the annotations for variant positions”
RKRRGSGAPVKQTLNFDLLKLAGDVESNPGP

SEQ ID NO: 50 - protein, 25 aa, Artificial Sequence
  source: /note=“Description of Artificial Sequence Synthetic peptide”
  VARIANT (5)..(7): /replace=“ ”
  SITE (5)..(7): /note=“This region may or may not be present in its entirety”
  SITE (1)..(25): /note=“Variant residues given in the sequence have no preference with respect to those in the annotations for variant positions”
RKRRGSGEGRGSLLTCGDVEENPGP